(19) United States
(12) Patent Application Publication
(10) Pub. No.: US 2024/0408239 A1
(43) Pub. Date: Dec. 12, 2024

(54) COMPOSITIONS AND METHODS TO PREVENT, INHIBIT OR TREAT NEURODEGENERATIVE DISEASES

(71) Applicants: The Trustees of Columbia University in the City of New York, New York, NY (US); Universitetet i Oslo, Oslo (NO)

(72) Inventors: Ai Yamamoto, New York, NY (US); Katherine R. Croce, New York, NY (US); Anne Gjøen Simonsen, Oslo (NO); Serhiy Pankiy, Gjettum (NO)

(21) Appl. No.: 18/666,480

(22) Filed: May 16, 2024

Related U.S. Application Data
(63) Continuation of application No. PCT/US2022/079957, filed on Nov. 16, 2022.
(60) Provisional application No. 63/280,070, filed on Nov. 16, 2021.

(51) Int. Cl. A61K 48/00; A61K 9/127; A61K 38/17; A61P 25/28

(52) U.S. Cl. CPC A61K 48/0058; A61K 9/127; A61K 38/1709; A61P 25/28

(57) ABSTRACT

Methods to prevent, inhibit or treat a neurodegenerative disease, e.g., one having protein aggregates, as well as compositions useful in that regard, are provided.

### CROSS-REFERENCE TO RELATED APPLICATIONS

This application claims the benefit of priority to PCT application No. PCT/US2022/079957, filed Nov. 16, 2022, which claims the benefit of priority to U.S. application No. 63/280,070, filed on Nov. 16, 2021, the disclosures of which are incorporated by reference herein.

### STATEMENT OF GOVERNMENT RIGHTS

This invention was made with government support under grants NS077111, NS101663, NS063973, and NS050199 awarded by the National Institutes of Health. The government has certain rights in the invention.

### INCORPORATION BY REFERENCE OF SEQUENCE LISTING

A Sequence Listing is provided herewith as an xml file, “2413153US1.xml” created on Nov. 16, 2022 and having a size of 49,858 bytes. The content of the xml file is incorporated by reference herein in its entirety.

### BACKGROUND

Although the lysosome-mediated degradation pathway autophagy has been implicated as a key player in the pathogenesis of neurodegenerative disease, whether and how autophagy might modify pathogenesis remains unclear. For example, Huntington's disease (HD) is an autosomal dominantly heritable neurological disorder characterized by motor disturbances, psychiatric changes and cognitive decline, caused by a trinucleotide repeat expansion mutation within the coding region of HD (Gusella et al., 1993). The resulting protein, mutant huntingtin (mHtt), aggregates and accumulates, leading to a pathological hallmark of the disease (Scherzinger et al, 1997; DiFiglia et al., 1997; Vonsattel et al., 2011).

It is well-established that the age of HD onset is inversely correlated with the length of the CAG expansion mutation. Early studies attributed the length of the CAG repeat to contribute to 80% of onset variability {Andrew et al., 1993; Gusella et al., 2000). These studies included the full range of reported mutations, some of which are quite rare (Wexler et al., 2004, Gusella et al., 2014). A subsequent re-analysis of the most frequently occurring mutations lengths in the Venezuelan HD kindred and others, revealed that the CAG repeat length contributes much less, leaving room for other environmental and genetic factors to play a role (Wexler et al., 2004; Gusella et al., 2009).

Protein aggregation is a molecular hallmark of many forms of neurodegeneration, including Huntington's Disease (HD) and Parkinson's Disease (PD), where large protein aggregates result in neurotoxicity, loss of neurons and the emergence of clinical symptoms that worsen over time. Autophagy refers to the process by which a cell consumes its own macromolecular structures to regulate itself or survive under stressful conditions. Aggrephagy is a special form of autophagy that targets protein aggregates.

### SUMMARY

Genetic studies from the Venezuelan Huntington's Disease cohort revealed a single nucleotide polymorphism (SNP) in the gene WDFY3 associated with a significant delay in the residual age of disease onset. As disclosed herein, introduction of the SNP in mice is sufficient to recapitulate its protective effects in patients, and delays the onset of behavioral and neuropathological symptoms in HD mice. The SNP acts by increasing expression of the gene product Alfy, an adaptor protein for the degradation of aggregated protein by selective autophagy (Simonsen, 2004; Eenjes, 2016; Filimonenko, 2010; Fox, 2020). Augmenting aggregate turnover is protective not only in HD mice, but also in a preformed fibril model of Parkinson's disease (PD). These findings indicate that diminishing the proteinopathy is protective across different neurodegenerative diseases. Thus, rs17368018 delays age of onset in HD patients.

Wdfy3 (Alfy) is a key regulator of aggrephagy process and clears protein aggregates. The present disclosure provides evidence that a single base change in the coding sequence of Alfy increases its expression and results in the clearance (breaking down) of more protein aggregates, e.g., increased clearance of protein aggregates, such as huntingtin (specific to HD) or alpha-synuclein (specific to PD) or phospho-tau (Alzheimer's disease). Following intrastriatal injections in mouse models of neurodegenerative disease, Alfy curtailed the propagation and escalation of aggregates in the brain, along with other benefits to symptom onset and survival.

Thus, overexpression of Alfy or a portion thereof, such as C-terminal Alfy, which may include residues 2461 to 3526 and/or a BEACH and FYVE domain, which is sufficient to facilitate aggregate clearance, in a mammal, such as a human, may prevent, inhibit or treat one or more symptoms of neurodegenerative diseases or other diseases involving protein aggregation, e.g., protein aggregation is the key driver of neurodegeneration in the brain and has been an intractable target of therapeutics for diseases such as Huntington's Disease (HD) and Parkinson's Disease (PD). Alfy is a protein that is directly involved in breaking down protein aggregates and maintaining homeostatic protein levels.

The disclosure thus provides a method to prevent, inhibit or treat a proteinopathy in a mammal, comprising administering to the mammal a composition an effective amount of isolated nucleic acid encoding Alfy or a portion thereof, a vector comprising a nucleotide sequence encoding Alfy or a portion thereof, or isolated Alfy or a portion thereof. In one embodiment, the mammal is a human. In one embodiment, the mammal has or is at risk of having Huntington's disease, Parkinson's disease, Lou Gehring's disease, or a disease associated with an aberrant TDP43 or aberrant expression thereof. In one embodiment, the Alfy or portion thereof has at least one amino acid substitution that if present in full length Alfy results in a variant Alfy that enhances clearance of protein aggregates relative to an Alfy without the one or more substitutions. In one embodiment, the variant enhances clearance of alpha synuclein, phospho-tau or TDP43. In one embodiment, the vector is a viral vector or a set of viral vectors. In one embodiment, the set of viral vectors each comprises a coding region for a portion of Alfy. In one embodiment, each portion is linked to a N-terminal or C-terminal intein. In one embodiment, each portion having a coding region is flanked by a splice acceptor site or a splice donor site, or both. In one embodiment, the viral vector or set of vectors comprise adeno-associated virus, adenovirus, lentivirus or a herpesvirus. In one embodiment, the isolated nucleic acid comprises a long non-coding RNA (LncRNA) or a corresponding DNA sequence. In one embodiment, the composition is administered to the central nervous system. In one embodiment, the composition is systemically administered. In one embodiment, the isolated nucleic acid comprises DNA. In one embodiment, a heterologous promoter is operably linked to DNA encoding Alfy or portion thereof. In one embodiment, the isolated nucleic acid comprises RNA. In one embodiment, the RNA comprises a plurality of modified nucleotides. In one embodiment, the RNA is sgRNA. In one embodiment, the composition comprises liposomes or nanoparticles. In one embodiment, the composition is sustained release composition.

Further provided is a method to prevent, inhibit or treat one or more symptoms of Alzheimer's disease, Amyotrophic Lateral Sclerosis, Frontotemporal dementia, parkinsonism-17, Frontotemporal lobar degeneration, Parkinson's disease, Huntington's disease, or Spinocerebellar ataxia type 3 in a mammal comprising: administering to the mammal a composition an effective amount of isolated nucleic acid encoding Alfy or a portion thereof, a vector comprising a nucleotide sequence encoding Alfy or a portion thereof, or isolated Alfy or a portion thereof. In one embodiment, the mammal is a human. In one embodiment, the Alfy or portion thereof has at least one amino acid substitution that if present in full length Alfy results in a variant Alfy that enhances clearance of protein aggregates relative to an Alfy without the one or more substitutions. In one embodiment, the variant enhances clearance of alpha synuclein, phospho-tau or TDP43. In one embodiment, the vector is a viral vector or a set of viral vectors. In one embodiment, the set of viral vectors each comprises a coding region for a portion of Alfy. In one embodiment, each portion is linked to a N-terminal or C-terminal intein. In one embodiment, each portion having a coding region is flanked by a splice acceptor site or a splice donor site. In one embodiment, the viral vector or set of vectors comprise adeno-associated virus, adenovirus, lentivirus or a herpesvirus. In one embodiment, the isolated nucleic acid comprises a long non-coding RNA (LncRNA) or a corresponding DNA sequence. In one embodiment, the composition is administered to the central nervous system. In one embodiment, the composition is systemically administered. In one embodiment, the isolated nucleic acid comprises DNA. In one embodiment, the isolated nucleic acid comprises RNA. In one embodiment, the RNA comprises a plurality of modified nucleotides.

In addition, a method to prevent, inhibit or treat a neurodegenerative disease having protein aggregates in a human is provided. In one embodiment, the disclosure provides for delivery of a gene editing system, e.g., CRISPR/Cas, TALENs, zinc finger nuclease or homing endonucleases (mega nucleases), or prime editing guide RNA (pegRNA) employed with, for example, a catalytically impaired Cas endonuclease such as Cas9 H840A nickase fused to a reverse transcriptase or base editing which employs a catalytically impaired Cas protein such as one fused to, for example, a deaminase converting A/T to G/C (adenine base editors although editors for other bases are envisioned, see, e.g., Table 1 in Rees and Liu, Nat.Rev.Genet., 19:770 (2019), the disclosure of which is incorporated by reference herein), delivered via one or more vectors such as plasmids or viral vectors or other delivery vehicles. The method includes administering to the human an effective amount of i) Cas or an isolated nucleic encoding Cas, and ii) isolated nucleic acid for one or more sgRNAs or pegRNAs comprising a targeting sequence for human Alfy genomic DNA. In one embodiment, liposomes or nanoparticles comprise Cas or the isolated nucleic acid encoding Cas. In one embodiment, liposomes or nanoparticles comprise the one or more sgRNAs or pegRNAs. In one embodiment, liposomes or nanoparticles comprise Cas or the isolated nucleic acid encoding Cas and the one or more sgRNAs or pegRNAs. In one embodiment, a viral vector comprises the isolated nucleic acid encoding Cas. In one embodiment, a viral vector comprises the one or more sgRNAs. In one embodiment, the one or more sgRNAs comprise SEQ ID NO:6 or a nucleic acid sequence having at least 80% or 90% nucleic acid sequence identity thereto or a nucleic acid sequence having 1, 2, 3, 4, 5 or 6 nucleotide substitutions relative to SEQ ID NO:6.

Further provided is a composition comprising isolated nucleic acid for one or more sgRNAs or pegRNAs comprising a targeting sequence for human Alfy genomic DNA and Cas or an isolated nucleic encoding Cas. In one embodiment, the one or more sgRNAs or pegRNAs comprise SEQ ID NO:6 or a nucleic acid sequence having at least 80% or 90% nucleic acid sequence identity thereto or a nucleic acid sequence having 1, 2, 3, 4, 5 or 6 nucleotide substitutions relative to SEQ ID NO:6. In one embodiment, the targeting sequence includes a nucleotide sequence having at least one amino acid substitution at a position from 3025 to 3037 in human Alfy. In one embodiment, position 3032 has a valine. In one embodiment, amino acid changes in one or more of amino acid residues 3346 to 3349, the LIR domain, may enhance binding to GABARAPS (Lystad, 2014). Furthermore, increased phosphorylation may increase autophagy-related interactions thereby augment function.

### BRIEF DESCRIPTION OF THE FIGURES

FIG. 1. Alfy is an adaptor that is essential for the clearance of aggregated proteins by MA. It is a 400 kDa protein with known motifs localized to the C-terminus of the protein. A LIR domain (which interacts specifically with the Atg8 ortholog GABARAP is found within the WD40 domain. Nuclear export (NES) and nuclear localization signals are noted along with the protein-protein and protein-lipid interactions domains.

FIGS. 2A-2C. HaloTag (HT) stable cell based assay for aggregation. Tetramethylrhodamine (TMR, red) or Oregon Green (OG, green). A. Differential labeling of aggregates by sequential addition of fluorophores. At time 0, TMR is placed on all cells followed by OG at times indicated. B. TMR aggregates clear while new OG aggregates increase in number. A pool of ‘growing aggregates’ that are TMR and OG positive is also present. C. Increasing Alfy decreases aggregate number. The decrease is due to a significant effect on TMR or OG-only aggregates, but not on the TMR-OG positive aggregates (n.s.; p=0.1823). Cells transfected with AlfyC or Ctrl construct were exposed to TMR to label pre-existing protein. 48 to 72 hrs later, OG was added to label newly expressed proteins. Cells were analyzed for TMR-only, TMR-OG and OG-only. Data in B, C are represented as Mean+s.d.mice/genotype/sex).*: p<0.001 FIGS. 3A-3D. Alfy KO, iKO and iKi mice. A, B. Gene trap insertion eliminates Alfy expression (1). C,D. Conditional Alfy deletion (1) via Cre, such as a tam-inducible Cre, ActinCreERTM. D. 10 mo old forebrain 1 wk after final Tam injection. A significant loss of Alfy both by in situ hybridization and western. Excision is achieved after i.p. administration of tam for five consecutive days.

FIGS. 4A-4D. Alfy is necessary for aggregate clearance in brain. A. Creation of HDAlfy. Mice were given either vehicle (veh) or tam to KO Alfy. To turn off Htt expression mice were given dox. B. Experimental design. C. Representative images of EM48 staining of striatum. D. Stereologic analysis of aggregate load. The clearance of EM48-positive structures observed in neurons, is significantly abrogated upon the loss of Alfy. ANOVA: Overall effect of Alfy iKO (F(1,20)=7.032, p=0.01) and an interaction between presence of Alfy and dox treatment (F(1,2)=4.001, p=0.03). n=4-5 mice per genotype per age. * represents HD mice (2 m/o: F(1,6)=8.632, p=0.02; 4 mo: F(1,8)=6.347, p=0.03). 45

FIGS. 5A-5D. Alfy depletion in HD neurons increases aggregation. A. Schematic representation of using brain-enriched microRNAs, miR-9/9*-124, to directly con-vert human fibroblasts into cells analogous to medium spiny neurons (MSNs) when combined with striatum-enriched factors, CTIP2, DLX1, DLX2, and MYT1L (CDM). B. Representative image of converted MSNs from fibroblasts isolated from a 60-yr old symptomatic HD patient (46Q) at post-induction day (PID) 19, immunestained for TUBB3 (green) and DAPI (blue). C-D. Immunostaining and quantification for mutant HTT inclusion bodies by MW8 antibody at PID19 in Control-MSNs or HD-MSNs transduced with shALFY or shCtrl. Dotted line boxes are shown to depict origin of HTT/DAPI insets.

FIGS. 6A-6C. Haploinsufficiency for Alfy accelerates accumulation of mutant Htt and behavioral deficits. A. Western blotting of 8M urea soluble, detergent insoluble Htt at 6 months. Compared to BACHD, there is a significant increase in the accumulation of mutant Htt in Alfy depleted mice (n=3/genotype). B. IHC for mutant Htt aggregates. At 12 mo, BACHD mice show few aggregates (2), but the depletion of Alfy makes them readily detectable (n=3/genotype). C. Rotarod (shown) and OpenField (not shown) reveals that Alfy depletion significantly accelerates progression (p<0.001) in males (shown) and females (not shown). Alfy deletion alone has no effect (n=12 to 15 mice/genotype/sex).

FIGS. 7A-7B. Competing models of selective MA. A. Current model suggests different cargoes are similarly identified by adaptor proteins such as p62, Nbr1 or Optn, then interact with LC3 found on the isolation membrane. B. The alternative model is that some or all of the adaptor proteins are part of a core complex shared by different cargoes. Specificity adaptors, such as Alfy are used for added specificity. For the sake of simplicity, the core adaptor proteins are depicted together, but it is conceivable that each contributes a distinct function: One adaptor might sequester cargo, and another might interact with LC3 on the outer membrane to stabilize AV formation.

FIGS. 8A-8D. Immunoisolation for purified AVs. A. AV isolation by gradient centrifugation leads to a significant enrichment of LC3 II. B. Immunoisolation of GFP-LC3 positive AVs. GFP-LC3 enrichment can be followed via confocal microscopy of each fraction. Immunoblotting shows how isolation for GFP-LC3 using magnetic beads eliminates probable contaminants such as ER. C, D. CryoEM analysis of purified AVs. C. Method in (A) alone leads to predominantly unilamellar structures (uni), suggesting contaminating structures such as lysosomes and microsomes. Imaging the crude prep after incubation with beads reveals that multilamellar (multi) structures may be labelled. This is confirmed when imaging is performed after immunnoisolation. D. The different types of structures from large complex multilamellar (1-4) to small unilamellar (5) structures.

FIGS. 9A-9G. Introduction of the SNP rs17368018 into the mouse genome recapitulates the delayed age of onset of the HD-like phenotype. A. Graphic representation of the pedigree identified in the HD Venezuelan cohort. Each individual is represented by a shape which is halved to show the HD positivity to the left, and the presence of the SNP to the right. Under each individual is the patient ID, and the residual AO (+/−years). Square, male; Circle, female; Blue, later than expected AO; Red, earlier than expected AO; hatched, rs17368018+; empty, rs17368018. B. Sequence alignment of the region of human WDFY3 and murine Wdfy3 in which rs17368018 is found (SEQ ID NOs:11-20). These two exons share 83.23% sequence identity. Grey indicates sequence differences in mouse. * indicate the site of the SNP (WT sequence shown). The SNP encodes a A to G changes within this coding exon changing the Isoleucine (Iso) to Valine (Val). C. Open Field Maze. Total locomotor behavior for 2 hrs during the dark cycle. ANOVA reveal significantly different locomotor behavior across genotype (F(1,87)=2.087, p<0.001). Post hoc analyses revealed that Q140 mice are significantly hypolocomotive versus control (female, p<0.001; male p=0.002), whereas Q140 mice expressing the Alfy Var are not. Female: Ctrl (AlfyVar/+) n=7, Q140 n=16, Q140::Alfy Var n=18; Male: Ctrl n=16, Q140 n=15, Q140::Alfy Var n=21. *, p<0.05. D-F. Neuropathological staining for reactive astrocytosis (GFAP), microgliosis (Iba1), and the medium spiny neuron marker, FoxP1. ANOVA reveals significant differences in the presence of Alfy Var. G. MAB5492 mHtt staining of striatum. ANOVA reveals significantly fewer mHtt aggregates in the presence of one or two copies of the Alfy variant.

FIGS. 9H-9J. Recreating SNP rs17368019 in mice. H. Genome wide association. I. Schematic representation of breeding the AlfyVar mice into the Q140 mouse model to create experimental animals. J. Locomotor analyses of control groups indicate the introduction of the A>G mutation does not alter locomotor behavior of male or female mice compared to control. ANOVA analyses of total distance traveled revealed no significant difference across groups (F(2,44)=0.697; p=0.5033), and although sex was considered separate variable a priori, no significant difference across sex (F(1,44)=3.874; p=0.0554). Females: WT n=12, AlfyVar/+n=4, AlfyVarNar n=4; Males: WT n=14, AlfyVar/+n=7, AlfyVar/Var n=9.

FIGS. 10A-10N. Ectopically increasing expression levels of Alfy is sufficient to recapitulate the protective effect of rs17368019. A-C. The presence of rs17268019 is associated with increased levels of WDFY3 expression in both A. transcript and B-C. protein. D-F. Introduction of the A>G variant in mice, reproduces the increased levels of Wdfy3 expression of both transcript (D) and protein (E-F) in mice. G-I. Ectopic overexpression of WDFY3 cDNA in mice. G-H: protein levels. I: Breeding schema to ectopically overexpress Alfy in Q140 mice. J-N. Ectopic overexpression of Alfy leads to a delayed onset of the hypolocomotor phenotype of the 6 m/o Q140 mouse (J), as well as preventing accumulation of aggregated mutant htt, (K), reactive astrocytosis (L), activated microgliosis (M), and decreased expression of the MSN marker, FoxP1 (N). J. ANOVA of total distance traveled revealed a significant difference across genotype (F(3,100)=7.176, p<0.001), no difference across sex (F(1,100)=1.060; p=0.3057), and no interaction between the two variables (F(3,100)=0.054, p=0.9834). Posthoc analyses reveals a significant difference between Ctrl mice and Q140 mice (Females, p=0.0023; Males, p=0.0039), but no significance with Q140 mice overexpressing Alfy as indicated on the figure. * indicates p<0.05. No significant difference was observed in between one or two doses of hAlfy overexpression (Females, p=0.5905;Males, p=0.6609). Females: Ctrl n=15, Q140 n=16, Q140:RosahAlfy/+, n=14, Q140::RosahAlfy/hAlfy, n=12; Males: Ctrl n=13, Q140 n=13, Q140::RosahAfy/+, n=10, Q140hAlfy/hAlfy n=15). Ctrl mice used are mice with one or two copies of hAlfy.

FIGS. 10O-10T. hAlfy overexpression rescues for the loss of murine Alfy. 0. Chromosome 4. P. Probing for FLAG indicates successful expression of a FLAG tagged protein on the correct predicted size. Q-S. Ectopic overexpression of hAlfy in mice as created, rescues the perinatal lethality and midline crossing deficits in the Alfy KO mice (Q). R. Nissl stained coronal sectin of a P90 forebrain shows the rescue preventing the lack of forebrain commissures, as previously reported to occur in the absence of Alfy. S. PCR genotyping of ear biopsies from P21 mice (Samples 1, 2, 3) for the presence of Alfy (Alfy), the hALfy allele (Rosa26hAlfy) and the floxed stop cassette (Rosa26flox-stop). Sample 3 is a P21 Alfy KO, who has survived due to the presence of the hAlfy allele, which is expressing since the flox-stop cassette is removed. T. Locomotor analyses of control groups indicate that hAlfy overexpression does not alter locomotor behavior of male or female mice through open field assays. ANOVA analyses of total distance traveled revealed no significant difference across groups (F(1,50)=0.327; p=0.5698), and although sex was considered separate variable a priori, no significant difference across sex (F(1,50)=1.002; p=0.3216). Females: WT n=12, RosahAlfy/hAlfy n=15; Males: WT n=14, RosahAlfy/hAlfy n=13.

FIGS. 11A-11F. The Alfy variant or ectopic overexpression of Alfy confers protection to the N171-82Q (N171) mouse. a.-d. Neuropathological images from striata of N171 mice. A. mHtt aggregate load. ANOVA reveals a significant difference across genotype (F(=, p=). Posthoc analyses reveals crossing with AlfyVar/+ or RosahAlfy/+ is significantly different from N171 (p=or p=, respectively). B. Reactive astrocytosis. ANOVA reveals a significant difference across genotype (F(2,6)=54.160, p<0.001). Posthoc analyses reveals crossing with Alfy^{Var/+} or Rosa^{hAlfy/+} is significantly different from N171 (p<0.001 for both). c. Activated microgliosis. ANOVA reveals a significant difference across genotype (F(2,6)=22.349, p=0.00²). Posthoc analyses reveals crossing with Alfy^{Var/+} or Rosa^{hAlfy/+} is significantly different from N171 (p=0.001 or p=0.002, respectively). d. Medium spiny neuron's FoxP1 expression levels. ANOVA reveals a significant difference across genotype (F(2,6)=12.089, p=0.008). Posthoc analyses reveals crossing with Alfy^{Var/+} or Rosa^{hAlfy/+} is significantly different from Ctrl (p=0.006 or p=0.005, respectively). E. Spontaneous locomotor activity. ANOVA analyses of total distance traveled revealed a significant effect of genotype when N171 mice were crossed with Alfy^{Var/Var} (F_{(2,59)}=10.953; p<0.0001), as with. While Fisher PLSD revealed that N171 became significantly hypolocomotive (p=0.0003), the presence of the variant delayed the appearance of this phenotype. Similar findings in the presence of ectopic overexpression with Rosa^{hAlfy/+}(F_{(2,79)}=7.199; p=0.0013). Again only N171 mice become significantly hypolocomotive (p=0.009). Posthoc analyses results as indicated. Effect of variant: Ctrl n=11, N171 n=11, N171::Alfy_{Var/Var} n=13; Effect of ectopic overexpression Ctrl n=20, N171 n=11, N171::Rosah^{Alfy/+} n=14. F. Survival curve reveals a significant delay of premature mortality upon increasing Alfy expression levels. AlfyVar/Var: Mann-Whitney U for time: Z=−6.4, p<0.001. RosahAlfy/+: Mann-Whitney U for time: Z=−4.524, p<0.001. N171 n=66; N171::AlfyVarNar n=29; N171::RosahAlfy/+n=61.

FIGS. 11G-11K. Increasing Alfy levels also delays disease onset in a fragment model of HD (N171 model). G. Breeding schema for the creation of N171-82Q (N171) mice, expressing either the Alfy variant or ectopic hAlfy. H. Diminished aggregate load in the cortex of N171 mice in the presence of the Alfy variant or Alfy overexpression. I. Locomotor analyses of control groups indicate that the Alfy variant or hAlfy overexpression does not alter locomotor behavior of mice compared to controls. ANOVA analyses of total distance traveled revealed no significant difference across groups when looking at the expression of the Alfy variant (Alfy Var: F(1,20)=0.310; p=0.86), or overexpression of hAlfy (Alfy OE: F(1,32)=3.298, p=0.089). Only males were analyzed. Alfy Var: WT n=10, AlfyVar/Var n=11; Alfy OE: WT n=10, RosahAlfy+n=10. J. Alfy overexpression delays the onset of rotarod deficits in the N171 (HD) mice. Male mice demonstrate a more consistent rotarod deficits than females. p-values generated from RM-ANOVA of the multiple trials per age, and posthoc analyses are listed below each line graph. Females: at 2 and 3m/o n=11 mice/genotype, at 4 m/o n=11 Ctrl, n=10 HD, n=10 HD::Alfy OE. Males: at 2 and 3 m/o n=11 mice/genotype, 4 m/o n=11 Ctrl, n=10 HD, n=9 HD::Alfy OE, 5 m/o n=11 Ctrl, n=7 HD, n=6 HD:Alfy OE. K. Overexpression of another autophagy gene does not augment lifespan in the N171 mice. Survival curve of N171 mice in the presence or absence of co-expression of GFP-LC3. Mann-Whitney U for time reveals no significant difference between the two groups. Z=−0.905, p=0.3655. N171 n=66, N171::GFP-LC3 n=9. Neuropathological images from striata of N171 mice a. Aggregate load. ANOVA reveals a significant difference across genotype (F(2,8)=17.805, p=0.003). Posthoc analyses reveals crossing with AlfyVar/+ or RosahAlfy/+ is significantly different from N171 (p=0.013 or p=0.013, respectively).

FIGS. 12A-12G. Increasing Alfy levels also protects against alpha-synuclein accumulation and neurotoxicity in a PFF model of synucleinopathy. a. Schematic representation of the model used. 6 m/o WT or RosahAlfy/hAlfy mice received a single injection of mouse aSYN pre-formed fibrils into the striatum. Mice were assessed as 120 days post-injection. b. Summarized representation of site of injection in the striatum C-D. Images and quantification of accumulated phospho(Ser129) alpha-synuclein (pS129-aSyn) from the substantia nigra pars compacta (SNpc). Quantification reveals that increasing basal levels of Alfy leads to significantly fewer aggregates per field (2 sided Student t-test, p<0.001). E-G Images and quantification of tyrosine hydroxylase-positive dopaminergic neurons (TH₊) and Nissl₊ neurons in the SNpc. Cell numbers contralateral (C) and ipsilateral (I) to the injection site were quantified. Similar to the protection conferred against aggregation, increased levels of Alfy led to significant protection against neuronal loss. Repeated measures (RM) ANOVA revealed a significant effect of genotype on TH counts (F(1,6)=8.398, p=0.027). i. RM-ANOVA revealed a significant effect of genotype on total Nissl counts (F(1,6)=14.405, p=0.009).

FIGS. 12H-12J. Increasing Alfy levels also slows the appearance of aggregates in projection areas in a PFF model of a Synucleinopathy. H. Phospho-synuclein aggregation in striatum. quantification reveals no difference of aggregate number in the injected field. Similar to the SNpc, projection areas such as the cortex (I) and amygdala (J) appear to have fewer aggregates when striatal injections were performed in the RosahAlfy/hAlfy mouse. n=4 WT, n=4 RosahAlfy/hAlfy.

FIGS. 13A-13C. Increasing Alfy levels confers protection by promoting ribosomal biogenesis in an HD background. A-C. Increasing Alfy levels does not significantly alter the transcriptome-wide signature of HD toxicity. A. Scatterplot of Z-statistic for differentially expressed (DE) genes between Q140 and WT colony from this study (y-axis) compared to those from a Q140 and Q20 comparison (x-axis) (Langfelder et al (2017)). Each point represents an independent single gene (n=15,809). Genome wide correlation coefficient of the Z-statistics indicate high correlation, and related correlated p-value indicate that the transcriptional signature of the Q140 is maintained. Similar comparison between Q140 and WT with Q140 and Q140hALfy/+(B) or Q140hALfy/hALfy (C) reveal little to no correlation, indicative that the presence of increased levels of Alfy does not return the transcriptional signature back to wildtype levels. Concordant and discordant genes that exceed the FDR threshold of 0.1 are indicative of genes whose expression were rescued (blue) and exacerbated (orange), respectively, and are listed in Extended Data Table xx. An equal number of 6 m/o male and female mice for a n=8/genotype.

FIGS. 13D-13H. Numbers of DE genes (FDR<0.1) across comparisons. Blue/orange indicate down-/up-regulated genes. D-F. Volcano plots examining the transcription wide changes evoked by Alfy overexpression in a WT (D) vs. Q140 (E-F). Although several gene changes were noted when Alfy was overexpressed in a WT background, no discernable pattern within these changes were observed using independent approaches. In the Q140 background, an Alfy dose-dependent transcriptional level change is observed that lead to two discrete changes as indicated by STRING analysis (G-H). Using the homozygous DE gene lists, analyses reveal the decreased gene expression predominantly leads to changes in NOTCH signaling and vascularization (G), whereas increased gene expression leads to changes in ribosomes and ribosomal biogenesis (H).

FIG. 14. Increasing WDFY3-AS2 levels increases levels of Alfy. RT-PCR of HELA cells transfected with the long non-coding RNA (lncRNA) WDFY3-AS2 (AS2). AS2 transient transfection led to a significant increase of Alfy expression relative to mock transfected (Ctrl) cells. Actin was used as a loading control. (student's t-test, p=0.028, n=3/condition).

FIGS. 15A-15C. Overexpression of Alfy prevents accumulation of Phopho-TDP43 inclusions, and protects against motor deficits. A. A transgenic mouse model that overexpresses human TDP-43 demonstrates phospho-TDP43 (pTDP43) positive inclusions throughout the cortex. When these mice are crossed to a mouse model that over-expresses one or two copies of Alfy, the aggregate-burden is diminished in a dose-dependent manner. B. Alfy overexpression rescues TDP43 accumulation in the superior olivary complex (TDP43^{+/+} on left and TDP43^{+/+}::Rosa/+ on right). There was an overall significant reduction in pTDP43 aggregation, a decrease in cytoplasmic tangles, a decrease in nuclear staining and rescue of nuclear pathology. C. Alfy overexpression impacts pTDP43 accumulation in cerebellum (TDP43^{+/+} on left and TDP43^{+/+}::Rosa/+ on right). The figure shows a reduction in nuclear staining in Purkinje cells (red arrows positive—white arrows negative) in TDP43^{+/+}::Rosa/+.

FIG. 15D. Hindlimb clasping phenotype in 9-month-old (m/o) TDP43 mice is diminished upon Alfy overexpression. TDP43 mice crossed to mice that ectopically overexpress 1 or 2 copies of Alfy or the Alfy variant show significant protection against this phenotype. TDP43 (n=6), TDP43::Rosa^{hAlfy/+}(n8) TDP43::Rosa^{hAlfy/hAlfy} (n=5), TDP43::Alfy^{Var/Var} (n=6).

FIGS. 16A-16B. A. Schematic presentation of in vivo 13032V mutation experiment of ALFY/WDFY3 using base editing (SEQ ID NOs:21-25). B. Genomic sequence of human ALFY/WDFY3 and examples of Sanger sequencing reads of wild type and 13032V mutants of ALFY/WDFY3 (SEQ ID NOs:26 and 27).

FIG. 17. Alfy overexpression protects against phospho-tau accumulation in a mouse model of tauopathy (P301S). P301S mice were crossed to mice overexpressing Alfy and assessed at 5 months of age. At this age, P301S mice show profound phospho-tau accumulation as shown by immunostaining against AT8 (brown), indicative of tau tangles. P301S mice expressing Alfy showed protection against tangle accumulation, as well as neuroprotection (Nissl-positive cells stained in blue).

### DETAILED DESCRIPTION

Although a common theme across adult-onset neurodegenerative diseases, the pathogenic role of aggregated proteins is a continuous topic of debate. For the incurable familial neurodegenerative disorder Huntington's disease (HD), resolving the accumulation of mutant huntingtin (Htt) (neuronal or cytoplasmic) is highly correlated with favorable therapeutic outcomes. Whether targeting aggregate clearance per se is beneficial, however, has remained unclear. A pathway was identified by which aggregated proteins are selectively eliminated by the lysosome-mediated pathway macroautophagy. It was found that the protein Alfy is central for the selective turnover of aggregates in cell-based systems. As described herein, genetic and molecular based approaches were employed to determine if augmenting Alfy levels promotes the elimination of aggregated nuclear and cytoplasmic proteins, the mechanism by which a genetic variant of Alfy might delay the age of onset of MD, and the molecular mechanism by which Alfy permits aggregate clearance.

Protein aggregation may be at the root cause of much of the pathology associated with adult-onset neurodegenerative disease. To determine if this is the case, it is determined if the turnover of aggregates can ameliorate diseases such as Huntington's disease in mice and more generally to determine if modulating autophagy impacts basal cellular function, or can modify pathogenesis in diseases ranging from Huntington's disease, Parkinson's disease and most recently Amyotrophic lateral sclerosis. The high degree of specialization of neural cells can lead to unexpected adaptations of a pathway. Moreover, the cellular needs of an embryonic brain may be quite distinct from an aging one.

A genetically modified mouse which inducibly expresses a form of the mutant huntingtin protein was employed. It was found that the constitutive expression of the mutant transgene was important not only for disease onset, but also for progression. Most surprising was that elimination of gene expression not only halted progression but led to the reversal of the disease-like phenotype. On a cellular level, it was revealed that neurons have an innate capacity to eliminate the abnormal proteinaceous inclusions that are a hallmark of this disease.

Although the pathogenicity of these abnormally accumulated proteins is an ongoing topic of debate, it remains to be determined if aggregate-clearance can be accomplished in vivo. A series of inducible cell lines that express, in a tet-regulatable manner, fluorophore-tagged polyglutamine proteins, were used to conduct a series of genetic and compound-based screens. It was found that macroautophagy is the primary means by which accumulated mutant huntingtin can be eliminated by the cell. What was particularly interesting was that the clearance of protein aggregates was independent of mTOR inhibition-indicative that not all macroautophagy cargo is eliminated in response to starvation, and a harbinger of the differential regulation of selective autophagy.

Macroautophagy cargo can range from cytosol to protein oligomers to intact organelles and pathogens. At the core of this versatility is the transient organelle known as the autophagosome. It is a de novo synthesized double membrane vesicle that forms around the cargo destined for elimination.

The present disclosure defines the requirements for the selective elimination of aggregated proteins, primarily the molecular contribution of the selectivity adaptor Autophagy Linked FYVE containing protein (Alfy) towards the selective degradation of aggregated proteins. It was found that whereas the loss of Alfy significantly impedes the clearance of expanded polyglutamine proteins and alpha-synuclein, its over expression can increase the rate at which these accumulated proteins are eliminated. Moreover, Alfy-mediated degradation can be modulated without affecting macroautophagy as a whole, making Alfy a powerful molecular target for controlling protein accumulation. Thus, Alfy and macroautophagy might play an essential role in the cellular remodeling events that can influence both neurodevelopment and disease.

### Definitions

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by hybridization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle is one that comprises a polynucleotide component which it is capable of delivering into a cell for which the viral species is trophic. The term does not necessarily imply any replication capacity of the virus.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment described herein that is a polynucleotide encompasses both the double-stranded form and each of two complementary single-stranded forms known or predicted to make up the double-stranded form.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments of this disclosure are envisioned. Thus, for example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use in the present disclosure generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present disclosure are provided below.

“Host cells,” “cell lines,” “cell cultures,” “packaging cell line” and other such terms denote higher eukaryotic cells, such as mammalian cells including human cells, useful in the present disclosure, e.g., to produce recombinant virus or recombinant fusion polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells of the present disclosure are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less e.g., with 2 bases or less. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less or with 2 or less. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 85%, 90%, 95% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, e.g., at least 90 to 95 percent sequence identity, or at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

“Conservative” amino acid substitutions are, for example, aspartic-glutamic as polar acidic amino acids; lysine/arginine/histidine as polar basic amino acids;
leucine/isoleucine/methionine/Valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic; trp, tyr, phe.

The disclosure also envisions polypeptides with non-conservative substitutions. Non-conservative substitutions entail exchanging a member of one of the classes described above for another.

As used herein, “individual” (as in the subject of the treatment) means a mammal. Mammals include, for example, humans; non-human primates, e.g., apes and monkeys; and non-primates, e.g., dogs, cats, rats, mice, cattle, horses, sheep, and goats. Non-mammals include, for example, fish and birds.

The term “disease” or “disorder” are used interchangeably, and are used to refer to neurodegenerative or proteinopathy diseases or conditions.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, “inhibiting” means inhibition of further progression or worsening of the symptoms associated with the disorder or disease, and “preventing” refers to prevention of the symptoms associated with the disorder or disease.

As used herein, an “effective amount” or a “therapeutically effective amount” of an agent, e.g., a recombinant AAV encoding a gene product, refers to an amount of the agent that alleviates, in whole or in part, symptoms associated with the disorder or condition, or halts or slows further progression or worsening of those symptoms, or prevents or provides prophylaxis for the disorder or condition, e.g., an amount that is effective to prevent, inhibit or treat in the individual one or more symptoms.

In particular, a “therapeutically effective amount” refers to an amount effective, at dosages and for periods of time necessary, to achieve the desired therapeutic result. A therapeutically effective amount is also one in which any toxic or detrimental effects of the agent(s) are outweighed by the therapeutically beneficial effects.

### Exemplary Alfy Nucleic Acid and Amino Acid Sequences

In one embodiment, Alfy comprises a polypeptide having the sequence in NCBI Reference Sequence: NM_014991.6, e.g.,

(SEQ ID NO: 1)
MNMVKRIMGRPRQEECSPQDNALGLMHLRRLFTELCHPPRHMTQKEQEE
KLYMMLPVFNRVFGNAPPNTMTEKFSDLLQFTTQVSRLMVTEIRRRASN
KSTEAASRAIVQFLEINQSEEASRGWMLLTTINLLASSGQKTVDCMTTM
SVPSTLVKCLYLFFDLPHVPEAVGGAQNELPLAERRGLLQKVFVQILVK
LCSFVSPAEELAQKDDLQLLFSAITSWCPPYNLPWRKSAGEVLMTISRH
GLSVNVVKYIHEKECLSTCVQNMQQSDDLSPLEIVEMFAGLSCFLKDSS
DVSQTLLDDFRIWQGYNFLCDLLLRLEQAKEAESKDALKDLVNLITSLT
TYGVSELKPAGITTGAPFLLPGFAVPQPAGKGHSVRNVQAFAVLQNAFL
KAKTSFLAQIILDAITNIYMADNANYFILESQHTLSQFAEKISKLPEVQ
NKYFEMLEFVVFSLNYIPCKELISVSILLKSSSSYHCSIIAMKTLLKFT
RHDYIFKDVFREVGLLEVMVNLLHKYAALLKDPTQALNEQGDSRNNSSV
EDQKHLALLVMETLTVLLQGSNTNAGIFREFGGARCAHNIVKYPQCRQH
ALMTIQQLVLSPNGDDDMGTLLGLMHSAPPTELQLKTDILRALLSVLRE
SHRSRTVFRKVGGFVYITSLLVAMERSLSCPPKNGWEKVNQNQVFELLH
TVFCTLTAAMRYEPANSHFFKTEIQYEKLADAVRFLGCFSDLRKISAMN
VFPSNTQPFQRLLEEDVISIESVSPTLRHCSKLFIYLYKVATDSFDSRA
EQIPPCLTSESSLPSPWGTPALSRKRHAYHSVSTPPVYPPKNVADLKLH
VTTSSLQSSDAVIIHPGAMLAMLDLLASVGSVTQPEHALDLQLAVANIL
QSLVHTERNQQVMCEAGLHARLLQRCSAALADEDHSLHPPLQRMFERLA
SQALEPMVLREFLRLASPLNCGAWDKKLLKQYRVHKPSSLSYEPEMRSS
MITSLEGLGTDNVFSLHEDNHYRISKSLVKSAEGSTVPLTRVKCLVSMT
TPHDIRLHGSSVTPAFVEFDTSLEGFGCLFLPSLAPHNAPTNNTVTTGL
IDGAVVSGIGSGERFFPPPSGLSYSSWFCIEHFSSPPNNHPVRLLTVVR
RANSSEQHYVCLAIVLSAKDRSLIVSTKEELLQNYVDDFSEESSFYEIL
PCCARFRCGELIIEGQWHHLVLVMSKGMLKNSTAALYIDGQLVNTVKLH
YVHSTPGGSGSANPPVVSTVYAYIGTPPAQRQIASLVWRLGPTHFLEEV
LPSSNVTTIYELGPNYVGSFQAVCMPCKDAKSEGVVPSPVSLVPEEKVS
FGLYALSVSSLTVARIRKVYNKLDSKAIAKQLGISSHENATPVKLIHNS
AGHLNGSARTIGAALIGYLGVRTFVPKPVATTLQYVGGAAAILGLVAMA
SDVEGLYAAVKALVCVVKSNPLASKEMERIKGYQLLAMLLKKKRSLLNS
HILHLTFSLVGTVDSGHETSIIPNSTAFQDLLCDFEVWLHAPYELHLSL
FEHFIELLTESSEASKNAKLMREFQLIPKLLLTLRDMSLSQPTIAAISN
VLSFLLQGFPSSNDLLRFGQFISSTLPTFAVCEKFVVMEINNEEKLDTG
TEEEFGGLVSANLILLRNRLLDILLKLIYTSKEKTSINLQACEELVKTL
GFDWIMMFMEEHLHSTTVTAAMRILVVLLSNQSILIKFKEGLSGGGWLE
QTDSVLTNKIGTVLGFNVGRSAGGRSTVREINRDACHFPGFPVLQSFLP
KHTNVPALYFLLMALFLQQPVSELPENLQVSVPVISCRSKQGCQFDLDS
IWTFIFGVPASSGTVVSSIHNVCTEAVFLLLGMLRSMLTSPWQSEEEGS
WLREYPVTLMQFFRYLYHNVPDLASMWMSPDFLCALAATVFPFNIRPYS
EMVTDLDDEVGSPAEEFKAFAADTGMNRSQSEYCNVGTKTYLTNHPAKK
FVFDFMRVLIIDNLCLTPASKQTPLIDLLLEASPERSTRTQQKEFQTYI
LDSVMDHLLAADVLLGEDASLPITSGGSYQVLVNNVFYFTQRVVDKLWQ
GMFNKESKLLIDFIIQLIAQSKRRSQGLSLDAVYHCLNRTILYQFSRAH
KTVPQQVALLDSLRVLTVNRNLILGPGNHDQEFISCLAHCLINLHVGSN
VDGFGLEAEARMTTWHIMIPSDIEPDGSYSQDISEGRQLLIKAVNRVWT
ELIHSKKQVLEELFKVTLPVNERGHVDIATARPLIEEAALKCWQNHLAH
EKKCISRGEALAPTTQSKLSRVSSGFGLSKLTGSRRNRKESGLNKHSLS
TQEISQWMFTHIAVVRDLVDTQYKEYQERQQNALKYVTEEWCQIECELL
RERGLWGPPIGSHLDKWMLEMTEGPCRMRKKMVRNDMFYNHYPYVPETE
QETNVASEIPSKQPETPDDIPQKKPARYRRAVSYDSKEYYMRLASGNPA
IVQDAIVESSEGEAAQQEPEHGEDTIAKVKGLVKPPLKRSRSAPDGGDE
ENQEQLQDQIAEGSSIEEEEKTDNATLLRLLEEGEKIQHMYRCARVQGL
DTSEGLLLFGKEHFYVIDGFTMTATREIRDIETLPPNMHEPIIPRGARQ
GPSQLKRTCSIFAYEDIKEVHKRRYLLQPIAVEVFSGDGRNYLLAFQKG
IRNKVYQRFLAVVPSLTDSSESVSGQRPNTSVEQGSGLLSTLVGEKSVT
QRWERGEISNFQYLMHLNTLAGRSYNDLMQYPVFPWILADYDSEEVDLT
NPKTFRNLAKPMGAQTDERLAQYKKRYKDWEDPNGETPAYHYGTHYSSA
MIVASYLVRMEPFTQIFLRLQGGHFDLADRMFHSVREAWYSASKHNMAD
VKELIPEFFYLPEFLFNSNNFDLGCKQNGTKLGDVILPPWAKGDPREFI
RVHREALECDYVSAHLHEWIDLIFGYKQQGPAAVEAVNVFHHLFYEGQV
DIYNINDPLKETATIGFINNFGQIPKQLFKKPHPPKRVRSRLNGDNAGI
SVLPGSTSDKIFFHHLDNLRPSLTPVKELKEPVGQIVCTDKGILAVEQN
KVLIPPTWNKTFAWGYADLSCRLGTYESDKAMTVYECLSEWGQILCAIC
PNPKLVITGGTSTVVCVWEMGTSKEKAKTVTLKQALLGHTDTVTCATAS
LAYHIIVSGSRDRTCIIWDLNKLSFLTQLRGHRAPVSALCINELTGDIV
SCAGTYIHVWSINGNPIVSVNTFTGRSQQIICCCMSEMNEWDTQNVIVT
GHSDGVVRFWRMEFLQVPETPAPEPAEVLEMQEDCPEAQIGQEAQDEDS
SDSEADEQSISQDPKDTPSQPSSTSHRPRAASCRATAAWCTDSGSDDSR
RWSDQLSLDEKDGFIFVNYSEGQTRAHLQGPLSHPHPNPIEVRNYSRLK
PGYRWERQLVFRSKLTMHTAFDRKDNAHPAEVTALGISKDHSRILVGDS
RGRVFSWSVSDQPGRSAADHWVKDEGGDSCSGCSVRFSLTERRHHCRNQ
GQLFCQKCSRFQSEIKRLKISSPVRVCQNCYYNLQHERGSEDGPRNC,

### or NCBI Reference Sequence: NP_055806.2, e.g.,

(SEQ ID NO: 2)
mnmvkrimgr prqeecspqd nalglmhlrr lftelchppr hmtqkeqeek lymmlpvfnr
vfgnappntm tekfsdllqf ttqvsrlmvt eirrrasnks teaasraivq fleinqseea
srgwmlltti nllassgqkt vdcmttmsvp stlvkclylf fdlphvpeav ggaqnelpla
errgllqkvf vqilvklcsf vspaeelaqk ddlqllfsai tswcppynlp wrksagevlm
tisrhglsvn vvkyihekec lstcvqnmqq sddlspleiv emfaglscfl kdssdvsqtl
lddfriwqgy nflcdlllrl eqakeaeskd alkdlvnlit slttygvsel kpagittgap
fllpgfavpq pagkghsvrn vqafavlqna flkaktsfla qiildaitni ymadnanyfi
lesqhtlsqf aekisklpev qnkyfemlef vvfslnyipc kelisvsill kssssyhcsi
iamktllkft rhdyifkdvf revgllevmv nllhkyaall kdptqalneq gdsrnnssve
dqkhlallvm etltvllqgs ntnagifref ggarcahniv kypqcrqhal mtiqqlvlsp
ngdddmgtll glmhsappte lqlktdilra llsvlreshr srtvfrkvgg fvyitsllva
merslscppk ngwekvnqnq vfellhtvfc tltaamryep anshffktei qyekladavr
flgcfsdirk isamnvfpsn tqpfqrllee dvisiesvsp tlrhcsklfi ylykvatdsf
dsraeqippc ltsesslpsp wgtpalsrkr hayhsvstpp vyppknvadl klhvttsslq
ssdaviihpg amlamldlla svgsvtqpeh aldlqlavan ilqslvhter nqqvmceagl
harllqrcsa aladedhslh pplqrmferl asqalepmvl reflrlaspl ncgawdkkll
kqyrvhkpss lsyepemrss mitsleglgt dnvfslhedn hyriskslvk saegstvplt
rvkclvsmtt phdirlhgss vtpafvefdt slegfgclfl pslaphnapt nntvttglid
gavvsgigsg erffpppsgl sysswfcieh fssppnnhpv rlltvvrran sseqhyvcla
ivlsakdrsl ivstkeellq nyvddfsees sfyeilpcca rfrcgeliie gqwhhlvlvm
skgmlknsta alyidgqlvn tvklhyvhst pggsgsanpp vvstvyayig tppaqrqias
lvwrlgpthf leevlpssnv ttiyelgpny vgsfqavcmp ckdaksegvv pspvslvpee
kvsfglyals vssltvarir kvynkldska iakqlgissh enatpvklih nsaghlngsa
rtigaaligy lgvrtfvpkp vattlqyvgg aaailglvam asdveglyaa vkalvcvvks
nplaskemer ikgyqllaml lkkkrslins hilhltfslv gtvdsghets iipnstafqd
llcdfevwlh apyelhlslf ehfielltes seasknaklm refqlipkll ltlrdmslsq
ptiaaisnvl sfllqgfpss ndllrfgqfi sstlptfavc ekfvvmeinn eekldtgtee
efgglvsanl illrnrlldi llkliytske ktsinlqace elvktlgfdw immfmeehlh
sttvtaamri lvvllsnqsi likfkeglsg ggwleqtdsv ltnkigtvlg fnvgrsaggr
stvreinrda chfpgfpvlq sflpkhtnvp alyfllmalf lqqpvselpe nlqvsvpvis
crskqgcqfd ldsiwtfifg vpassgtvvs sihnvcteav flllgmlrsm ltspwqseee
gswlreypvt lmqffrylyh nvpdlasmwm spdflcalaa tvfpfnirpy semvtdldde
vgspaeefka faadtgmnrs qseycnvgtk tyltnhpakk fvfdfmrvli idnlcltpas
kqtplidlll easperstrt qqkefqtyil dsvmdhllaa dvllgedasl pitsggsyqv
lvnnvfyftq rvvdklwqgm fnkeskllid fiiqliaqsk rrsqglslda vyhclnrtil
yqfsrahktv pqqvalldsl rvltvnrnli lgpgnhdqef isclahclin lhvgsnvdgf
gleaearmtt whimipsdie pdgsysqdis egrqllikav nrvwtelihs kkqvleelfk
vtlpvnergh vdiatarpli eeaalkcwqn hlahekkcis rgealapttq sklsrvssgf
glskltgsrr nrkesglnkh slstqeisqw mfthiavvrd lvdtqykeyq erqqnalkyv
teewcqiece llrerglwgp pigshldkwm lemtegpcrm rkkmvrndmf ynhypyvpet
eqetnvasei pskqpetpdd ipqkkparyr ravsydskey ymrlasgnpa ivqdaivess
egeaaqqepe hgedtiakvk glvkpplkrs rsapdggdee nqeqlqdqia egssieeeek
tdnatllrll eegekiqhmy rcarvqgldt seglllfgke hfyvidgftm tatreirdie
tlppnmhepi iprgarqgps qlkrtcsifa yedikevhkr ryllqpiave vfsgdgrnyl
lafqkgirnk vyqrflavvp sltdssesvs gqrpntsveq gsgllstlvg eksvtqrwer
geisnfqylm hintlagrsy ndlmqypvfp wiladydsee vdltnpktfr nlakpmgaqt
derlaqykkr ykdwedpnge tpayhygthy ssamivasyl vrmepftqif lrlqgghfdl
adrmfhsvre awysaskhnm advkelipef fylpeflfns nnfdlgckqn gtklgdvilp
pwakgdpref irvhrealec dyvsahlhew idlifgykqq gpaaveavnv fhhlfyegqv
diynindplk etatigfinn fgqipkqlfk kphppkrvrs ringdnagis vlpgstsdki
ffhhldnlrp sltpvkelke pvgqivctdk gilaveqnkv lipptwnktf awgyadlscr
lgtyesdkam tvyeclsewg qilcaicpnp klvitggtst vvcvwemgts kekaktvtlk
qallghtdtv tcataslayh iivsgsrdrt ciiwdlnkls fltqlrghra pvsalcinel
tgdivscagt yihvwsingn pivsvntftg rsqqiicccm semnewdtqn vivtghsdgv
vrfwrmeflq vpetpapepa evlemqedcp eaqigqeaqd edssdseade qsisqdpkdt
psqpsstshr praascrata awctdsgsdd srrwsdqlsl dekdgfifvn ysegqtrahl
qgplshphpn pievrnysrl kpgyrwerql vfrskltmht afdrkdnahp aevtalgisk
dhsrilvgds rgrvfswsvs dqpgrsaadh wvkdeggdsc sgcsvrfslt errhhcrncg
qlfcqkcsrf qseikrlkis spvrvcqncy ynlqhergse dgprnc,

as well as a polypeptide with at least 80%, 85%, 90%, 95% or more, e.g., 99% or more, amino acid sequence identity thereto, or a portion thereof with the activity of SEQ ID NO:1 or SEQ ID NO:2.

An exemplary mRNA sequence for Alfy comprises:

agacagccag cgggaggtgg agaaagcagg aggaggagga ggattaaaga tggccaccaa
cagctgcggg aaacggcaac aacccctcac tttccgggat ggtccctgcg ggtcggcccg
gccttgatgg agagaagaaa cccgaggagc gccgaggctg aggcggcggc ggcggggacc
cagcgaggac gaggacgcgg cggagcaggg acgggggcag gagaagggaa aggcggcggc
gtcgctgccc ctgctgccta gcaccgctgc ctggcccggc ggaccggttc ccatacctcg
cggccgcaga atcgagctcg ggccccggcc cccggcccgc ggcgcggggc tcccgggccc
cgccgcggac gtcgcgccgg tcgccccttc cccgtagccc gtgcgccctc ggcgcggagc
cccggcccgc cgcggtcccg tctcctgggc ctgtcccgcc cgcgccctcc gccggccctc
aggtataata cttctccacg tctgcttcag gaagaaagtg cctgccattc ttatcatttc
taagcaggtt catgccagcc cagaacagag aatcagctgg agcccagatt tcaagttttg
agtaaaatac cttcaagcga atgggcccta ttgtgctcac acattcagaa cctgttaccc
aaggaattcc ctaaagaatt agaagtgcgt ctcaccaacc agccaagatg aacatggtga
agaggatcat ggggcggccg aggcaggagg agtgcagccc acaagacaac gccttaggac
tgatgcacct ccgccggctc ttcacggagt tgtgccatcc tccccggcac atgactcaga
aggaacaaga agagaaactg tatatgatgc tgccagtgtt taacagggtt tttggaaatg
ctccgccgaa tacaatgaca gaaaaatttt ctgatcttct gcagttcaca acacaagtct
cacgactaat ggtgacagaa attcgaagga gagcatcaaa caaatccaca gaggctgcaa
gtcgggccat agttcagttc ctagagatta atcagagtga agaagccagt agaggctgga
tgcttctaac gacaattaat ttgttagctt cctctggtca gaaaaccgtg gactgcatga
caacaatgtc agtgccttcc accctggtta aatgtttata tctgtttttt gaccttccac
atgtgcctga ggcagttgga ggtgcacaga atgagctacc tctagcagaa cgtcgaggac
tactccagaa agtttttgta cagatcttag tgaaactgtg cagttttgtt tcccctgcgg
aggagctggc tcagaaagat gatctccagc ttctattcag tgcaataacc tcttggtgcc
ctccctataa cctgccttgg agaaagagtg ctggagaagt cctcatgacc atatctcgtc
atggtcttag tgtcaatgta gtgaagtata ttcatgagaa agagtgttta tctacatgtg
ttcagaatat gcagcaatca gatgacctgt ctcccctaga aattgtcgaa atgtttgctg
ggctttcttg tttcctcaaa gattccagcg atgtttccca aacacttctg gatgattttc
ggatatggca aggatataat tttctttgtg atctcttgct tagattggaa caagcaaaag
aggcagaatc caaagatgcc ttgaaagatc tggttaatct gataacttcc ctaacaacat
atggtgtcag tgaactaaaa ccagctggta ttaccacagg ggcacccttt ttattgcctg
gatttgcagt acctcagcct gcaggcaaag gtcacagtgt gagaaacgtc caggcctttg
cagttcttca gaatgcattt ttaaaagcaa aaaccagctt ccttgcccaa atcatccttg
atgctatcac aaatatttac atggctgaca atgccaatta cttcatccta gagtcacagc
acacattgtc acagtttgca gagaagattt ctaaactccc agaagtacaa aacaaatact
ttgagatgct ggagtttgtt gtttttagct taaattatat accttgtaaa gaacttatta
gtgtcagtat cctcttaaaa tctagctctt cttatcactg tagcattatt gcaatgaaaa
cacttcttaa gtttacaaga catgactaca tatttaaaga cgtgttcagg gaggttggcc
ttttggaggt catggtaaac cttttgcata aatatgctgc cctgttgaag gatccaactc
aggcactaaa tgaacaaggg gactcaagaa ataatagttc agttgaagac caaaaacacc
tggctttatt ggttatggag accttgacag tgcttcttca aggatcaaac acaaatgcag
gaatttttcg agaatttgga ggtgcaagat gtgcacataa tatagtaaag taccctcaat
gccggcagca tgccttgatg actatccaac agctggtgct ctccccaaat ggggacgatg
acatgggcac tctcctgggg ctaatgcatt cagccccacc gacggaattg cagttgaaga
ctgatatttt aagggccctc ctgtcggtcc ttcgagaaag ccatcgttca agaacagttt
ttaggaaagt tggaggattt gtgtacatta catccttgct cgttgctatg gaaagatctt
tgagctgtcc acccaagaat ggctgggaga aagtgaacca gaatcaagtg tttgaacttc
ttcacactgt gttctgcacg ttgactgcag caatgcgcta tgagccagcc aactctcatt
tcttcaaaac agagattcag tatgagaagt tggcagatgc tgttcgattt cttggctgct
tctcagacct aagaaaaata agcgccatga atgtcttccc ctcaaataca cagccatttc
aaagactttt agaggaagat gtaatctcaa tagaatcagt gtcacccacg ttacggcact
gcagtaaact ttttatttat ctttacaaag tagccacaga ttcttttgac agtcgtgcag
aacagatccc tccttgcctg acaagtgagt cttctctccc ctctccttgg ggtacaccag
ctttgtccag gaaaaggcat gcatatcatt ctgtttcaac tccccctgtt taccctccta
aaaatgttgc cgacctgaaa ctacatgtga caacttcatc tctgcagagt tctgatgcag
tcatcattca tcctggagcc atgcttgcca tgctggacct actggcctct gttgggtcag
tgacacagcc agaacatgct ttggatcttc aacttgccgt ggcaaatatt ttacaatccc
tggtgcacac agaaaggaac cagcaagtca tgtgtgaagc tggtcttcat gcacgactgc
tgcagaggtg cagtgctgca ttggctgatg aggaccactc actgcacccg cccctgcagc
ggatgtttga acgattagcc tctcaggctc tggaacccat ggtgttgagg gagtttttac
gtttggcaag tcctttaaat tgtggtgcct gggacaaaaa actgctaaaa caatataggg
tccacaaacc aagttcactg agttatgaac cagaaatgag aagtagtatg atcacatctc
tggaaggtct gggtactgat aatgttttta gcttacatga agataaccat taccggataa
gcaagagcct ggtaaaatct gcggaaggaa gtactgtacc cctgaccagg gtgaagtgtc
tggtctccat gacaacccca catgacatca gacttcatgg gtcatcagtt actccagctt
ttgttgaatt tgacacatca cttgaagggt ttggatgtct ttttttgccc agtttggccc
ctcataatgc tcctacaaat aataccgtca caacaggtct tattgatggg gctgtggtca
gtggcattgg ttctggtgaa agattcttcc ctcctccctc cggcttaagt tactctagct
ggttttgtat tgaacatttt agttctcctc caaataacca ccctgtcaga cttcttactg
ttgtgcgccg agcaaattct tctgagcaac attacgtgtg ccttgcaata gttctatcag
caaaagaccg atctctgatt gtttccacca aagaggaact cctccaaaat tatgttgatg
attttagtga agagtcctca ttttatgaaa ttctcccatg ctgtgctcgc tttcgatgtg
gagagcttat cattgaggga cagtggcatc atttggtcct ggtaatgagc aaaggcatgt
tgaaaaacag tactgcagcc ctttatattg atggacagct tgttaacact gtaaagcttc
attatgtcca cagtactcca gggggttcag gttcggcaaa tccaccagtg gtgagcacgg
tctatgccta cattggtact ccacctgccc aacgccaaat tgcctcattg gtttggcgcc
tgggacccac acattttcta gaagaagttt taccttcttc aaatgttact accatttatg
aacttggacc aaattatgtt ggaagctttc aggctgtatg tatgccatgt aaagatgcaa
aatccgaagg ggtggtgcca tcccctgtgt cattagtacc agaggagaaa gtgtcatttg
gcctctatgc actctctgtg tcgtctctaa cagtggcaag aatccggaaa gtgtataaca
aattggatag caaagccatt gctaagcagt taggcatttc ctcacatgag aatgccactc
ctgtgaagtt gatacacaat tcagcaggac atcttaatgg atctgcacgg acaattgggg
ccgctctgat tggatacttg ggagtaagaa catttgtccc taagcctgtt gccactactt
tgcagtacgt tggtggagct gcagccatcc tgggcctggt ggccatggcc tctgatgtgg
aagggttata tgcagcagtc aaggccctgg tttgtgtggt caagagtaac ccactagcca
gcaaagaaat ggaaagaatc aagggctacc agttgctggc aatgttgctt aagaagaaac
gttcccttct taacagccac atcctccatc taactttttc tttggtggga actgttgata
gtggacatga gacctccatt attccaaatt caactgcttt ccaggacctc ctctgtgatt
ttgaagtctg gctccatgca ccatatgaac ttcatctttc cttatttgaa cactttattg
aactgctcac agagtccagt gaagcctcaa agaatgccaa attaatgaga gaattccagt
taatcccaaa gctgctcctg actcttcgag atatgtcttt atcccagcct actattgctg
ctattagtaa tgtcctgagc ttcttactgc aaggttttcc tagcagcaat gatctgctca
gatttgggca gtttatttct tctactttgc caacctttgc ggtttgtgag aaatttgtag
taatggaaat aaataatgaa gagaagcttg acactggaac tgaagaggag tttggaggtc
ttgtatcagc taatcttata cttttgagga acagacttct ggatatcttg ctaaaactaa
tttatacatc taaagaaaag acaagcatta atttgcaagc ttgtgaagaa ctggtgaaga
cactgggttt tgactggatc atgatgttta tggaggaaca cttacattcc accacagtta
cagcagccat gaggattctt gttgtcctac taagtaatca gtctattctc atcaagttta
aagaaggact cagtggtgga ggatggcttg aacagacaga ttctgtctta actaataaga
ttggaactgt attaggattc aacgtgggca gaagtgctgg tgggagatcg acggtcaggg
agattaaccg agatgcttgt cattttcctg gttttccagt ccttcagtca ttccttccta
aacacactaa tgtccctgcc ctctattttc tcctcatggc cttgtttctg cagcagccag
ttagtgagct gcctgagaac ctgcaggtca gtgtgcctgt catcagctgc cggagtaagc
agggttgcca gtttgatttg gattccattt ggacattcat ctttggagtt cctgcctcca
gcggaactgt ggtctcttct atccataacg tatgcacaga agctgttttt ttattattgg
gaatgctccg cagcatgctg acttcacctt ggcaatcaga agaagaggga tcttggctcc
gagaatatcc tgtgaccctg atgcagttct tcagatattt gtatcacaac gtgccagacc
ttgcctccat gtggatgagc cctgacttcc tgtgtgcatt agcagccacc gtcttcccct
tcaatattcg cccttactca gagatggtga ctgaccttga tgatgaagtt ggatctccag
cagaagagtt taaagcgttt gcagcagaca cagggatgaa caggagccaa tcagagtact
gcaatgtggg caccaagaca tatctgacca atcacccggc taaaaagttc gtttttgact
tcatgcgggt cttaatcata gacaacctct gtctcactcc tgccagcaag caaactccac
taattgatct tttgttggag gcttcccctg aaaggtctac aagaactcag caaaaagaat
ttcaaactta cattttggat agcgtgatgg accatttgct tgcagctgat gtgttattag
gggaagatgc atctctgcct attaccagtg gaggaagcta ccaggtattg gtgaacaatg
tgttttattt cacacagcgt gtggtggaca agctttggca aggcatgttc aacaaagaat
ctaaacttct tatagatttt ataattcaac taattgcaca gtcaaagaga agatcacagg
gattgtcact ggatgcagtg tatcattgcc tcaataggac catcttgtac cagttctcac
gggcacacaa aaccgttcct cagcaagtag ctctgcttga ttcactcagg gtcctcactg
taaacagaaa cttgatcctg ggacctggga accatgacca agaattcatt agctgtctgg
cccactgctt gataaatcta catgttggaa gcaacgtgga tggatttgga ctggaagcag
aagcccgcat gaccacatgg cacattatga tcccctcgga cattgaacca gatggtagtt
acagccaaga tattagtgaa gggcgtcagc ttctcataaa agctgtcaac agagtttgga
ctgaactgat acatagtaag aaacaagtct tagaggaact tttcaaagta actctacctg
tgaatgaaag gggccacgtg gacatagcta cagcaaggcc actcattgaa gaagctgccc
tgaagtgctg gcagaatcat ttggcccatg aaaagaaatg cataagtcga ggagaagctt
tagcgcccac cacacagtcc aaattatccc gtgtcagcag tggctttggt ctttccaagt
taacaggatc aagaaggaat cgaaaagaaa gtggtcttaa taaacacagt ctttccaccc
aggagatttc gcagtggatg tttactcaca ttgctgttgt tcgtgactta gtagatacac
aatataaaga atatcaggag cgtcagcaga atgccctgaa gtacgtgaca gaagagtggt
gtcagatcga gtgcgagctg ttgagggagc gggggctgtg gggccctccc atcggctccc
acctcgacaa gtggatgctg gagatgacag aagggccctg caggatgagg aaaaagatgg
tgcgaaatga tatgttttat aaccattacc cttacgtgcc agaaactgag caagagacaa
atgtggcgtc tgagatccca agtaaacagc ctgagacacc cgatgatatt cctcaaaaga
aacctgctcg atatagaaga gccgtaagtt atgacagtaa agagtactac atgcgactgg
cctctggcaa tcccgccatt gtccaagacg ccattgtgga gagttcagaa ggtgaagctg
ctcagcaaga accagagcat ggggaagaca ctattgctaa agtcaaaggt ttggtcaagc
ctcctctaaa acgctcccga tctgcacctg atggaggaga tgaggagaac caggagcagc
tacaagacca gattgctgag ggcagctcca tagaagagga ggagaaaaca gataatgcta
ccttactgcg cctgttagag gaaggagaaa agatccaaca catgtaccgc tgtgctcgag
tccagggcct agataccagt gaggggctcc ttctttttgg taaagagcat ttttatgtga
ttgatggatt taccatgaca gcaaccaggg aaataagaga tattgaaacc ttacctccaa
atatgcatga gcctattatt cctagaggag ccaggcaagg ccctagtcaa ctcaagagaa
catgcagcat ttttgcatat gaagatatca aggaagttca taaaaggaga tatctcctgc
agcctattgc tgtggaagtt ttctctggag atggacggaa ttacctcctt gcttttcaga
aaggaatcag aaacaaagtc tatcaaaggt ttttggctgt agtgccatct ctaacggaca
gttcagaatc tgtatctggg caacgaccaa acacgagtgt ggagcaggga tctgggttac
ttagcacttt ggttggagag aagtctgtga ctcagagatg ggagagaggt gaaatcagca
acttccaata tttgatgcat ttgaacactt tggctggcag atcatataat gatctcatgc
agtatcctgt cttcccctgg atccttgcag attatgactc agaggaggtg gatcttacta
atcccaagac gtttagaaac ctggctaagc caatgggagc acaaacagat gaacgattag
ctcagtataa gaagcggtat aaagactggg aggatcctaa tggagaaact cctgcatacc
actatgggac ccactattca tctgcaatga ttgtggcctc ataccttgta aggatggagc
ctttcacaca gatattctta aggctacagg gtggccactt tgacctggct gaccggatgt
ttcacagtgt gcgcgaggcc tggtattcag cgtcaaagca caatatggca gatgtaaaag
aacttatccc agagttcttt tatttaccag aattcctgtt caattccaac aactttgatc
taggctgtaa acaaaatggc accaagcttg gagatgttat ccttccaccc tgggcaaaag
gggacccacg agaattcatc agagtccatc gtgaggcttt ggagtgtgat tacgtgagtg
cccatctaca tgagtggatt gacttaatct tcggttataa acagcaaggc cctgctgcag
tagaagctgt aaatgtcttc catcatcttt tttatgaggg tcaagtggat atctacaaca
tcaatgaccc actaaaggag acagccacaa ttgggttcat taataacttc ggtcagatcc
ctaaacagtt atttaaaaaa cctcatccac caaagcgagt gagaagtcga ctcaatggag
acaatgcagg aatctctgtc ctaccaggat ctacaagtga caagatcttt tttcatcatc
tagacaactt gaggccttct ctaacacctg taaaagaact caaagaacct gtaggacaaa
tcgtatgtac agataaaggt attcttgcgg tggaacagaa taaggttctt atcccaccaa
cctggaataa aacttttgct tggggctatg cagacctcag ttgcagactg ggaacctatg
agtcagacaa ggccatgact gtttatgaat gcttgtctga gtggggccag attctctgtg
caatctgccc caaccccaag ctggtcatca cgggtggaac aagcacggtt gtgtgtgtgt
gggagatggg cacctccaaa gaaaaggcca agaccgtcac cctcaaacag gccttactgg
gccacactga taccgtcacc tgcgccacag catcattagc ctatcacata attgtcagtg
ggtcccgtga tcgaacctgt atcatttggg atttgaacaa actgtcattt ctaacccagc
ttcgagggca tcgagctcca gtttctgctc tttgtatcaa tgaattaaca ggggacattg
tgtcctgcgc tggcacatat atccatgtgt ggagcatcaa tgggaaccct atcgtgagtg
tcaacacgtt cacaggtagg agccagcaga tcatctgctg ctgcatgtcg gagatgaacg
aatgggacac gcagaacgtc atagtgacag gacactcaga tggagtggtt cggttttgga
gaatggaatt tttgcaagtt cctgaaacac cagctcctga gcctgctgaa gtcctagaaa
tgcaggaaga ctgtccagaa gcacaaatag ggcaggaagc ccaagacgag gacagcagtg
attcagaagc agatgagcag agcatcagcc aggaccctaa ggacactcca agccaaccca
gcagcaccag ccacaggccc cgggcagcct cctgccgcgc aacagccgcc tggtgtactg
acagtggctc tgacgactcc agacgctggt ccgaccagct cagtctagat gagaaagacg
gcttcatatt tgtgaactat tcagagggcc agaccagagc ccatctgcag ggccccctta
gccaccccca ccccaatccc attgaggtgc ggaattacag cagattgaaa cctgggtacc
gatgggaacg gcagctggtg ttcaggagta agctgactat gcacacagcc tttgatcgaa
aggacaatgc acacccagct gaggtcactg ccttgggcat ctccaaggat cacagtagga
tcctcgttgg tgacagtcga ggccgagttt tcagctggtc tgtgagtgac cagccaggcc
gttctgctgc tgatcactgg gtgaaggatg aaggtggtga cagctgctca ggctgctcgg
tgaggttttc actcacagaa agacgacacc attgcaggaa ctgtggtcag ctcttctgcc
agaagtgcag tcgctttcaa tctgaaatca aacgcttgaa aatctcatcc ccggtgcgtg
tttgtcagaa ctgttattat aacttacagc atgagagagg ttcagaagat gggcctcgaa
attgttgaag attcaacaag ctgagtggag accatggtct gtagacccct tcccgattct
cctgtcccag cttggaaggc attgaaaaca gtctccgttt acacatctct tcataccacg
tgtttgaagt gttaaaattc aaagggatca ttgaataaaa cgggtgtaga gtacaggaat
ggggcagacg cgattcaggt gaacagcaca agaagaatat gaggtggttc ctaggagcaa
cactttcgac ctccagttct ccctgatgac agtagctgtc tccaagagaa aaatcctcac
ttattaactc tcttttcttg catctcattt ttatagagct actcatcctt atttggaaaa
accaacaaca aaaaaggctt ttagaaaatg gttgtaaatc tgacttcttt gcaagtaact
atgtatattg taaatagata taaaaggcct tttttctaaa taaggactta actgcctgta
acatgaaact tcaaactaaa ccactaactc aatgaactac ttatggtttg tctgacatcc
ctcacttacc aattaattat aaatatgttt ttttaaatcc ccaaagacat tatctgtggt
ctttttttcc tttcaagctc agcctgtgtg cctgatgtca tttctttcaa gttgcccaca
gtatctccac ttaaactagg ctagtaacca aaataatgtg gaccttcttt aggaaacagt
gtgggagaat aggagtccag ccgtaagata aactggaaat atttgggcgt cttgtacctg
gctacgcacc acctcagtgt tgttcctaca taaacagggc cccttttaaa cttgtatgtg
gactgctgtt tggtcaaaga ataccttctt agcattgcag aaaggtggtc agatgaccag
tgtagtgcag gaaacagccc tgtctcaact aatggaaata tatttgcatg taacccaaaa
ttagcttatc ttgcatagaa cataataagt atgtgtcttt ggtgacacta atgttctact
atagcttatt ttcaaacaag gggtaaaaaa aggaaagaaa gaagtgtaca gaattaacat
ataaactttg ttgtaaaact gaatcatgtc agaactgctt aaaattaacc tttaccattt
aatgtcatct acctgaaaac agtgagattt atactgtatc aatgtctatt tttttgtttt
tgctatgaat ataattacag tattttaata tttagttatt taatttgttc tactagttgg
atacagaaca cacaaatcca gggggattaa agctggaagg ggctaagaga ttagtttaca
gagaaaaggc ttggtggtgg gattttttta aatgtgtgtt atgtacatat atatatatat
ataatatata ttaaaaatga aacaattaat ctagatttta acattttcag aaacttagtg
ataacattat gaacaattct aaaagccctg tgatttgaaa aatatagaat cattaatggc
ccaagatagg ccttcacacc ttcacaggtg cgaaaggaaa ggccttcaca ccctcacaga
ggcatcatgc aaaggacagc ggctttggct tttccaattt tccatcttta ggccctggtg
agaggcacac ttatgcacta aaatgcacat atatgcacat gcattcaaaa ataggcattt
ggtacaatgg tgatcttgta cctgatgggc tgaaaccagc ttaagaacaa atttgttctt
cctgatatga taactaggtc tccaagagaa aatagaaagg ctgctttagt gccttacgct
tactaaattt aaatctttat ttacctgggt ttgagcctac agtctattta tgattacata
tcaaaattga ttaaaacact tccatttcta aaagttcaaa tatacttgtt aataaaagga
ttatcggcat taatacttta atttaaagaa aagttgtgtt ctgttttcct ttctgtgtct
tactcccccc acactctccc tcccccatca ccatcttcaa ttctaataaa taatgctgat
gttcaacagt tgcagaaatt gtgctattat gtaactgtgg gccttgcccc tgtctggccc
tctagatgat ttgtagcagt gttattctac actttttaaa agaagcgtcc tccttttgtc
catgaatcat gtttacccca tacccagtgg cagaggtgtt ctttaaagac ttgaatatat
gaatgtgtgt gtgtagttac ttaaaggtta ttcctctttg taataggaaa ctatatggga
tgaacacttt taaactttcc gacacaactt ccattactaa ctttctaaca gaacttccat
aactagaagg tggaaaccaa aaccctcatg gtagtatttc ctctggcagc tggtgctgtg
ggcaactgtt ttgttcaatc gggtttcttt ttttttgcc tctaatgcag aaatcaacag
aatcactcac acatacaagt acactcacat acataaacta attatttctc tggatatctt
tctgtgttcc atgtaaattt atttaccaac atctattgtc aacatgtaca tctaccttag
tatggtctgc attctttttc tgagagtacc tcatagggct cctgcctgat ctttgtagtt
tgttcattca tccatccacc tgttcatttg ttcatccatg tattctaaca tttctatgta
gtgtgcaact ctaatgtcat gcttttgaag aagagaatag ctgcccatag cagccatccg
tctggataat agcaaaacac tctagataag ttattttgca ctttcttatg tataaagttg
gtagaaactt atttttgctt tgtatcattt aaatacattt tgttttggta aatgaactgt
gtataaaata tttatgccgt taaaactgtt tttagaaagt atttttaatt tcagcaagtt
tggttacttg ttgcatgact cttaacacag ctgacttttt gtgtcagtgc aatgtatatt
ttttgtcctg ttattaactt gtaagcccta gtaatggcca attatttgta cagcaacaga
agtaaattga agatactggc taagactgga ttgattgtgg acttttatac tatattgcag
aaaccaatat ctgtttcttg gtggttatgt aaaagacctg aagaattact atctagtgtg
cagtctgtga tatctgaatg ttcattgtat atttgtctct gatgcaaaaa ggtagagtaa

(SEQ ID NO:9), or a nucleotide sequence having at least 80%, 85%, 90%, 92%, 95%, 96%, 97%, 98%, 99% or more sequence identity thereto which in one embodiment encodes a polypeptide having SEQ ID NO:1 or 2, or a polypeptide with at least 80%, 85%, 90%, 95% or more, e.g., 99% or more, amino acid sequence identity thereto, or a portion thereof with the activity of SEQ ID NO:1 or SEQ ID NO:2.

### Delivery Vectors

Delivery vectors include, for example, nucleic acid, viral vectors, liposomes and other lipid-containing complexes, such as lipoplexes (DNA and cationic lipids), polyplexes, e.g., DNA complexed with cationic polymers such as polyethylene glycol, nanoparticles, e.g., magnetic inorganic nanoparticles that bind or are functionalized to bind DNA such as Fe₃O₄ or MnO₂ nanoparticles, microparticles, e.g., formed of polylactide polygalactide reagents, nanotubes, e.g., silica nanotubes, and other macromolecular complexes capable of mediating delivery of a gene or polypeptide, or a both, to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. A large variety of such vectors are known in the art and are generally available.

Gene delivery vectors include, but are not limited to, isolated nucleic acid, e.g., plasmid-based vectors which may be extrachromosomally maintained, isolated RNA, e.g., sgRNA, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary viral gene delivery vectors are described below. Gene delivery vectors may be administered via any route including, but not limited to, intracranial, intrathecal, intramuscular, buccal, rectal, intravenous or intracoronary administration, and transfer to cells may be enhanced using electroporation and/or iontophoresis, and/or scaffolding such as extracellular matrix or hydrogels, e.g., a hydrogel patch. In one embodiment, a permeation enhancer is not employed to enhance indirect delivery to the CNS.

### Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

### Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. Although lentiviruses have specific tropisms, pseudotyping the viral envelope with vesicular stomatitis virus yields virus with a broader range (Schnepp et al., Meth. Mol. Med., 69:427 (2002)).

### Adenoviral Vectors

Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells and, in particular, these vectors have been shown to efficiently infect cardiac myocytes in vivo, e.g., after direction injection or perfusion. Adenoviral vectors have been shown to result in transient expression of therapeutic genes in vivo, peaking at 7 days and lasting approximately 4 weeks. The duration of transgene expression may be improved in systems utilizing neural specific promoters. In addition, adenoviral vectors can be produced at very high titers, allowing efficient gene transfer with small volumes of virus.

### Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans.

AAV vectors include but are not limited to AAV1, AAV2, AAV5, AAV7, AAV8, AAV9 or AAVrh.10.

### Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. Plasmid DNA may be delivered to cells as part of a macromolecular complex, e.g., a liposome or DNA-protein complex, and delivery may be enhanced using techniques including electroporation.

Where translation is also desired in the intended target cell, the heterologous polynucleotide may also comprise control elements that facilitate translation (such as a ribosome binding site or “RBS” and a polyadenylation signal). Accordingly, the heterologous polynucleotide generally comprises at least one coding region operatively linked to a suitable promoter, and may also comprise, for example, an operatively linked enhancer, ribosome binding site and poly-A signal. The heterologous polynucleotide may comprise one encoding region, or more than one encoding regions under the control of the same or different promoters. The entire unit, containing a combination of control elements and encoding region, is often referred to as an expression cassette.

### The CRISPR/Cas System

The Type II CRISPR is a well characterized system that carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system. The primary products of the CRISPR loci appear to be short RNAs that contain the invader targeting sequences, and are termed guide RNAs.

“Cas1” polypeptide refers to CRISPR associated (Cas) protein1. Cas1 (COG1518 in the Clusters of Orthologous Group of proteins classification system) is the best marker of the CRISPR-associated systems (CASS). Based on phylogenetic comparisons, seven distinct versions of the CRISPR-associated immune system have been identified (CASS1-7). Cas1 polypeptide used in the methods described herein can be any Cas1 polypeptide present in any prokaryote. In certain embodiments, a Cas1 polypeptide is a Cas1 polypeptide of an archaeal microorganism. In certain embodiments, a Cas1 polypeptide is a Cas1 polypeptide of a Euryarchaeota microorganism. In certain embodiments, a Cas1 polypeptide is a Cas1 polypeptide of a Crenarchaeota microorganism. In certain embodiments, a Cas1 polypeptide is a Cas1 polypeptide of a bacterium. In certain embodiments, a Cas1 polypeptide is a Cas1 polypeptide of a gram negative or gram positive bacteria. In certain embodiments, a Cas1 polypeptide is a Cas1 polypeptide of Pseudomonas aeruginosa. In certain embodiments, a Cas1 polypeptide is a Cas1 polypeptide of Aquifex aeolicus. In certain embodiments, a Cas1 polypeptide is a Cas1 polypeptide that is a member of one of CASs1-7. In certain embodiments, Cas1 polypeptide is a Cas1 polypeptide that is a member of CASS3. In certain embodiments, a Cas1 polypeptide is a Cas1 polypeptide that is a member of CASS7. In certain embodiments, a Cas1 polypeptide is a Cas1 polypeptide that is a member of CASS3 or CASS7.

In some embodiments, a Cas1 polypeptide is encoded by a nucleotide sequence provided in GenBank at, e.g., GeneID number: 2781520, 1006874, 9001811, 947228, 3169280, 2650014, 1175302, 3993120, 4380485, 906625, 3165126, 905808, 1454460, 1445886, 1485099, 4274010, 888506, 3169526, 997745, 897836, or 1193018 and/or an amino acid sequence exhibiting homology (e.g., greater than 80%, 90 to 99% including 91%, 92%, 93%, 94%, 95%, 96%, 97%, 98% or 99%) to the amino acids encoded by these polynucleotides and which polypeptides function as Cas1 polypeptides.

There are three types of CRISPR/Cas systems which all incorporate RNAs and Cas proteins. Types I and Ill both have Cas endonucleases that process the pre-crRNAs, that, when fully processed into crRNAs, assemble a multi-Cas protein complex that is capable of cleaving nucleic acids that are complementary to the crRNA.

In type II CRISPR/Cas systems, crRNAs are produced using a different mechanism where a trans-activating RNA (tracrRNA) complementary to repeat sequences in the pre-crRNA, triggers processing by a double strand-specific RNase 11 in the presence of the Cas9 protein. Cas9 is then able to cleave a target DNA that is complementary to the mature crRNA however cleavage by Cas 9 is dependent both upon base-pairing between the crRNA and the target DNA, and on the presence of a short motif in the crRNA referred to as the PAM sequence (protospacer adjacent motif)). In addition, the tracrRNA must also be present as it base pairs with the crRNA at its 3′ end, and this association triggers Cas9 activity.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand.

The requirement of the crRNA-tracrRNA complex can be avoided by use of an engineered “single-guide RNA” (sgRNA) that comprises the hairpin normally formed by the annealing of the crRNA and the tracrRNA (see Jinek, et al. (2012) Science 337:816 and Cong et al. (2013) Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRN

“Cas polypeptide” encompasses a full-length Cas polypeptide, an enzymatically active fragment of a Cas polypeptide, and enzymatically active derivatives of a Cas polypeptide or fragment thereof. Suitable derivatives of a Cas polypeptide or a fragment thereof include but are not limited to mutants, fusions, covalent modifications of Cas protein or a fragment thereof.

### RNA Components of CRISPR/Cas

The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present (see Cong, et al. (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA). (see Jinek, ibid and Cong, ibid).

Chimeric or sgRNAs can be engineered to comprise a sequence complementary to any desired target. The RNAs comprise 22 bases of complementarity to a target and of the form G[n19], followed by a protospacer-adjacent motif (PAM) of the form NGG. Thus, in one method, sgRNAs can be designed by utilization of a known ZFN target in a gene of interest by (i) aligning the recognition sequence of the ZFN heterodimer with the reference sequence of the relevant genome (human, mouse, or of a particular plant species); (ii) identifying the spacer region between the ZFN half-sites; (iii) identifying the location of the motif G[N20]GG that is closest to the spacer region (when more than one such motif overlaps the spacer, the motif that is centered relative to the spacer is chosen); (iv) using that motif as the core of the sgRNA. This method advantageously relies on proven nuclease targets. Alternatively, sgRNAs can be designed to target any region of interest simply by identifying a suitable target sequence that conforms to the G[n20]GG formula. Donors

As noted above, insertion of an exogenous sequence (also called a “donor sequence” or “donor” or “transgene” or “gene of interest”), for example for correction of a mutant gene or for increased expression of a wild-type gene. It will be readily apparent that the donor sequence is typically not identical to the genomic sequence where it is placed. A donor sequence can contain a non-homologous sequence flanked by two regions of homology to allow for efficient HDR at the location of interest. Alternatively, a donor may have no regions of homology to the targeted location in the DNA and may be integrated by NHEJ-dependent end joining following cleavage at the target site. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang, et al. (1987) Proc. Natl. Acad. Sci. USA 84:4959-4963; Nehls, et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor is generally inserted so that its expression is driven by the endogenous promoter at the integration site, namely the promoter that drives expression of the endogenous gene into which the donor is inserted (e.g., highly expressed, albumin, AAVS1, HPRT, etc.). However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an albumin or other locus such that some (N-terminal and/or C-terminal to the transgene encoding the lysosomal enzyme) or none of the endogenous albumin sequences are expressed, for example as a fusion with the transgene encoding the lysosomal sequences. In other embodiments, the transgene (e.g., with or without additional coding sequences such as for albumin) is integrated into any endogenous locus, for example a safe-harbor locus. See, e.g., U.S. Patent Publication Nos. 2008/0299580; 2008/0159996; and 2010/0218264.

When endogenous sequences (endogenous or part of the transgene) are expressed with the transgene, the endogenous sequences (e.g., albumin, etc.) may be full-length sequences (wild-type or mutant) or partial sequences. The endogenous sequences may be functional. Non-limiting examples of the function of these full length or partial sequences (e.g., albumin) include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

### Exemplary Non-Viral Delivery Vehicles

Biodegradable particles comprising, e.g., isolated nucleic acid or a vector or a polypeptide, or a combination thereof, may include or may be formed from biodegradable polymeric molecules which may include, but are not limited to polylactic acid (PLA), polyglycolic acid (PGA), co-polymers of PLA and PGA (e., polyactic-co-glycolic acid (PLGA)), poly-ε-caprolactone (PCL), polyethylene glycol (PEG), poly(3-hydroxybutyrate), poly(p-dioxanone). polypropylene fumarate. poly(orthoesters), polyol/diketene acetals addition polymers, poly-alkyl-cyano-acrylates (PAC), poly(sebacic anhydride) (PSA), poly(carboxybiscarboxyphenoxyphenoxy hexone (PCPP) poly[bis (p-carboxyphenoxy),methane](PCPM), copolymers of PSA, PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, or gelatin. (See, e.g., Kumari et al, Colloids and Sur aces B: Biointerfaces 75 (2010) 1-18; and U.S. Pat. Nos. 6,913,767; 6,864,435; 6,565,777; 6,534,092: 6,528,087; 6,379,704; 6,309,569; 6,264,987; 6,210,707:6,090,925; 6,022,564; 5,981,719; 5,871,747; 5,723,269; 5,603,960; and 5,578,709; and US. Published Application No. 2007/0081972; and International Application Publication Nos. WO 2012/115806; and WO 2012/054425; the contents of which are incorporated herein by reference in their entireties).

The biodegradable nanoparticles may be prepared by methods known in the art, (See, e.g., Nagavarma et al., Asian J. of Pharma. And Clin. Res., Vol 5, Suppl 3, 2012, pages 16-23; Cismaru et al., Rev. Roum. Chim., 2010, 55(8), 433-442; and International Application Publication Nos. WO 2012/115806; and WO 2012/054425; the contents of which are incorporated herein by reference in their entireties). Suitable methods for preparing the nanoparticles may include methods that utilize a dispersion of a preformed polymer, which may include but are not limited to solvent evaporation, nanoprecipitation, emulsification/solvent diffusion, salting out, dialysis, and supercritical fluid technology. In some embodiments, the nanoparticles may be prepared by forming a double emulsion (e.g., water-in-oil-in-water) and subsequently performing solvent-evaporation. The nanoparticles obtained by the disclosed methods may be subjected to further processing steps such as washing arid lyophilization, as desired. Optionally, the nanoparticles may be combined with a preservative (e.g., trehalose).

Typically, the nanoparticles have a mean effective diameter of less than 1 micron, e.g., the nanoparticles have a mean effective diameter of between about 25 nm and about 500 nm, e.g., between about 50 nm and about 250 nm, about 100 nm to about 150 nm, or about 450 nm to 650 nm. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS), Nanoparticle Tracking Analysis (NTA), X-Ray Diffraction (XRD), Aerosol Time of Flight Mass Spectroscopy (ATFMS), and Aerosol Particle Mass Analyzer (APM).

The biodegradable nanoparticles may have a zeta-potential that facilitates uptake by a target cell, Typically, the nanoparticles have a zeta-potential greater than 0. In some embodiments, the nanoparticles have a zeta-potential between about 5 mV to about 45 mV. between about 15 mV to about 35 mV. or between about 20 mV and about 40 mV. Zeta-potential may be determined via characteristics that include electrophoretic mobility or dynamic electrophoretic mobility. Electrokinetic phenomena and electroacoustic phenomena may be utilized to calculate zeta-potential.

In one embodiment, a non-viral delivery vehicle comprises polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(β-amino ester), chitosan, PEI-polyethylene glycol, PEI-mannose-dextrose, DOTAP-cholesterol or RNAiMAX.

In one embodiment, the delivery vehicle is a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units yielded exceptional delivery efficiency.

In one embodiment, the delivery vehicle comprises polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM, or any combination thereof. The disclosed cationic polymer may include, but are not limited to, polyamidoamine (PAMAM) dendrimers. Polyamidoamine dendrimers suitable for preparing the presently disclosed nanoparticles may include 3rd-. 4th 5th, or at least 6th-generation dendrimers.

In one embodiment, the delivery vehicle comprises a lipid, e.g., N-[1-(2,3-dioleoyloxy)propel]-N,N,N-trimethylammonium (DOTMA), 2,3-dioleyloxy-N-[2-spermine carboxamide] ethyl-N,N-dimethyl-1-propanaminium trifluoracetate (DOSPA, Lipofectamine); 1,2-dioleoyl-3-trimethylammonium-propane (DOTAP); N-[1-(2,3-dimyristoyl) propyl]; N,N-dimethyl-N-(2-hydroxyethyl) ammonium bromide (DMRIE), 3-β-[N—(N,N-dimethylaminoethane) carbamoyl] cholesterol (DC-Chol); dioctadecyl amidoglyceryl spermine (DOGS, Transfectam); or dimethyldioctadecylammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008; van der Woude et al., 1997; Ilies et al., 2004). In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of cationic polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing cationic liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although complex formation with PLL may also increase the encapsulation efficiency. Other cationic materials, for example, PEI, DOTMA, DC-Chol, or CTAB, may be used to make nanospheres.

In one embodiment, complexes are embedded in or applied to a material including but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the biocompatible material is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the disclosure may be from a combination of sources. Isolated ECM may be prepared as a sheet, in particulate form, gel form and the like.

The biocompatible scaffold polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer may be formed of any of a wide range of materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan hydrogels.

### Pharmaceutical Compositions

The disclosure provides a composition comprising, consisting essentially of, or consisting of the above-described vector and/or isolated nucleic acid and/or isolated polypeptide, and a pharmaceutically acceptable (e.g., physiologically acceptable) carrier. When the composition consists essentially of the vector and/or isolated nucleic acid and/or isolated polypeptide, and a pharmaceutically acceptable carrier, additional components can be included that do not materially affect the composition (e.g., adjuvants, buffers, stabilizers, anti-inflammatory agents, solubilizers, preservatives, etc.). When the composition consists of the vector and/or isolated nucleic acid and/or isolated polypeptide, and the pharmaceutically acceptable carrier, the composition does not comprise any additional components. Any suitable carrier can be used within the context of the disclosure, and such carriers are well known in the art. The choice of carrier will be determined, in part, by the particular site to which the composition may be administered and the particular method used to administer the composition. The composition optionally can be sterile with the exception of the gene transfer vector described herein. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the inventive gene transfer vector is administered in a composition formulated to protect the gene transfer vector from damage prior to administration. For example, the composition can be formulated to reduce loss of the vector and/or isolated nucleic acid and/or isolated polypeptide, on devices used to prepare, store, or administer the gene transfer vector, such as glassware, syringes, or needles. The composition can be formulated to decrease the light sensitivity and/or temperature sensitivity of the vector and/or isolated nucleic acid and/or isolated polypeptide. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the vector, and/or isolated nucleic acid and/or isolated polypeptide, facilitate administration, and increase the efficiency of the i method. Formulations for gene transfer vector-containing compositions are further described in, for example, Wright et al., Curr. Opin. Drug Discov. Devel., 6(2): 174-178 (2003) and Wright et al., Molecular Therapy, 12:171-178 (2005))

The composition also can be formulated to enhance transduction efficiency. In addition, one of ordinary skill in the art will appreciate that the vector and/or isolated nucleic acid and/or isolated polypeptide, can be present in a composition with other therapeutic or biologically-active agents. For example, factors that control inflammation, such as ibuprofen or steroids, can be part of the composition to reduce swelling and inflammation associated with in vivo administration of the gene transfer vector. Immune system stimulators or adjuvants, e.g., interleukins, lipopolysaccharide, and double-stranded RNA. Antibiotics, i.e., microbicides and fungicides, can be present to treat existing infection and/or reduce the risk of future infection, such as infection associated with gene transfer procedures.

Injectable depot forms are made by forming microencapsule matrices of the subject compounds in biodegradable polymers such as polylactide-polyglycolide. Depending on the ratio of active agent to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the inventive gene transfer vector. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the vector and/or isolated nucleic acid and/or isolated polypeptide, in the composition administered to the mammal will depend on a number of factors, including the size (mass) of the mammal, the extent of any side-effects, the particular route of administration, and the like. In one embodiment, the inventive method comprises administering a “therapeutically effective amount” of the composition comprising the vector and/or isolated nucleic acid and/or isolated polypeptide, described herein. A “therapeutically effective amount” refers to an amount effective, at dosages and for periods of time as necessary, to achieve a desired therapeutic result. The therapeutically effective amount may vary according to factors such as the extent of the disease or disorder, age, sex, and weight of the individual, and the ability of the gene transfer vector to elicit a desired response in the individual.

The dose of vector in the composition to achieve a particular therapeutic effect typically is administered in units of vector genome copies per cell (gc/cell) or vector genome copies/per kilogram of body weight (gc/kg). One of ordinary skill in the art can readily determine an appropriate vector dose range to treat a patient having a particular disease or disorder, based on these and other factors that are well known in the art. The therapeutically effective amount may be between 1×10¹⁰ genome copies to 1×10¹³ genome copies. The therapeutically effective amount may be between 1×10¹¹ genome copies to 1×10¹⁴ genome copies. The therapeutically effective amount may be between 1×10⁷ genome copies to 1×10¹⁰ genome copies. The therapeutically effective amount may be between 1×10¹⁴ genome copies to 1×10¹⁷ genome copies. Assuming a 70 kg human, the dose ranges may be from 1.4×10⁸ gc/kg to 1.4×10¹¹ gc/kg, 1.4×10⁹ gc/kg to 1.4×10¹² gc/kg, 1.4×10¹⁰ gc/kg to 1.4×10¹³ gc/kg, or 1.4×10¹¹ gc/kg to 1.4×10¹⁴ gc/kg.

The nucleic acids or vectors, or polypeptides, may be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results.

In one embodiment, the composition is administered once to the mammal. It is believed that a single administration of the composition may result in persistent expression in the mammal with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

The present disclosure provides pharmaceutically acceptable compositions which comprise a therapeutically-effective amount of vector and/or isolated nucleic acid and/or isolated polypeptide, as described above.

### Routes of Administration, Dosages and Dosage Forms

Administration of, for example, the vectors and/or isolated nucleic acid and/or isolated polypeptide, in accordance with the present disclosure, may be continuous or intermittent, depending, for example, upon the recipient's physiological condition, and other factors known to skilled practitioners. The administration of the vector(s) and/or isolated nucleic acid and/or isolated polypeptide, may be essentially continuous over a preselected period of time or may be in a series of spaced doses. Both local administration, e.g., intracranial, intranasal or intrathecal, and systemic administration, e.g., using viruses that cross the blood-brain barrier, are contemplated. Any route of administration may be employed, e.g., intravenous, intranasal or intrabronchial, direct administration to the lung and intrapleural. In one embodiment, compositions may be delivered to the pleura.

One or more suitable unit dosage forms comprising the vector(s), and/or isolated nucleic acid and/or isolated polypeptide, which may optionally be formulated for sustained release, can be administered by a variety of routes including intracranial, intrathecal, or intranasal, or other means to deliver to the CNS, or oral, or parenteral, including by rectal, buccal, vaginal and sublingual, transdermal, subcutaneous, intravenous, intramuscular, intraperitoneal, intrathoracic, or intrapulmonary routes. The formulations may, where appropriate, be conveniently presented in discrete unit dosage forms and may be prepared by any of the methods well known to pharmacy. Such methods may include the step of bringing into association the vector with liquid carriers, solid matrices, semi-solid carriers, finely divided solid carriers or combinations thereof, and then, if necessary, introducing or shaping the product into the desired delivery system.

The amount of vector(s) and/or isolated nucleic acid and/or isolated polypeptide, administered to achieve a particular outcome will vary depending on various factors including, but not limited to, the genes and promoters chosen, the condition, patient specific parameters, e.g., height, weight and age, and whether prevention or treatment, is to be achieved.

Vectors and/or isolated nucleic acid and/or isolated polypeptide, may conveniently be provided in the form of formulations suitable for administration, e.g., into the brain. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

Vectors and/or isolated nucleic acid and/or isolated polypeptide, may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The vectors and/or isolated nucleic acid and/or isolated polypeptide, can be provided in a dosage form containing an amount of a vector effective in one or multiple doses. For viral vectors, the effective dose may be in the range of at least about 10⁷ viral particles, e.g., about 10⁹ viral particles, or about 10¹¹ viral particles. The number of viral particles added may be up to 10¹⁴. For example, when a viral expression vector is employed, about 10⁸ to about 10⁶⁰ gc of viral vector can be administered as nucleic acid or as a packaged virion. In some embodiments, about 10⁹ to about 10¹⁵ copies of viral vector, e.g., per 0.5 to 10 mL, can be administered as nucleic acid or as a packaged virion. Alternatively, the nucleic acids, polypeptides or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results. The amount administered will vary depending on various factors including, but not limited to, the nucleic acid or vector chosen for administration, the disease, the weight, the physical condition, the health, and/or the age of the mammal. Such factors can be readily determined by the clinician employing animal models or other test systems that are available in the art. As noted, the exact dose to be administered is determined by the attending clinician, but may be in 1 mL phosphate buffered saline. For delivery of plasmid DNA alone, or plasmid DNA or RNA, e.g., in a complex with other macromolecules, the amount of DNA to be administered will be an amount which results in a beneficial effect to the recipient. For example, from 0.0001 to 1 mg or more, e.g., up to 1 g, in individual or divided doses, e.g., from 0.001 to 0.5 mg, or 0.01 to 0.1 mg, of DNA or RNA can be administered.

For example, when a viral expression vector is employed, about 10⁸ to about 10⁶⁰ gc of viral vector can be administered as nucleic acid or as a packaged virion. In some embodiments, about 10⁹ to about 10¹⁵ copies of viral vector, e.g., per 0.5 to 10 mL, can be administered as nucleic acid or as a packaged virion. Alternatively, the nucleic acids, polypeptides or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results.

In one embodiment, administration may be by intracranial, intraventricular, intracisternal, lumbar, intrahepatic, intratracheal or intrabronchial injection or infusion using an appropriate catheter or needle. A variety of catheters may be used to achieve delivery, as is known in the art. For example, a variety of general purpose catheters, as well as modified catheters, suitable for use in the present disclosure are available from commercial suppliers. Also, where delivery is achieved by injection directly into a specific region of the brain or lung, a number of approaches can be used to introduce a catheter into that region, as is known in the art.

By way of illustration, liposomes and other lipid-containing gene delivery complexes can be used to deliver one or more transgenes. The principles of the preparation and use of such complexes for gene delivery have been described in the art (see, e.g., Ledley, (1995); Miller et al., (1995); Chonn et al., (1995); Schofield et al., (1995); Brigham et al., (1993)).

### Subjects

The subject may be any animal, including a human. human and non-human animals. Non-human animals include all vertebrates, e.g., mammals and non-mammals, such as non-human primates, sheep, dogs, cats, cows, horses, chickens, amphibians, and reptiles, although mammals, such as non-human primates, sheep, dogs, cats, cows and horses may be the subject. The subject may also be livestock such as, cattle, swine, sheep, poultry, and horses, or pets, such as dogs and cats.

In one embodiment, subjects include human subjects. The subject is generally diagnosed with the condition by skilled artisans, such as a medical practitioner.

The methods of the disclosure described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, childrens, and infants.

Examples of human ethnic populations include Caucasians, Asians, Hispanics, Africans, African Americans, Native Americans, Semites, and Pacific Islanders. The methods of the disclosure may be more appropriate for some ethnic populations such as Caucasians, especially northern European populations, as well as Asian populations.

The term subject also includes subjects of any genotype or phenotype as long as they are in need of the disclosure, as described above. In addition, the subject can have the genotype or phenotype for any hair color, eye color, skin color or any combination thereof.

The term subject includes a subject of any body height, body weight, or any organ or body part size or shape.

The invention will be further described by the following non-limiting examples.

### Example 1

A variant of Alfy was identified that was significantly associated with a delayed AO by an average of ten years. Subsequently, it was found that the level of Alfy is significantly higher in brains from variant-positive individuals than variant-negative individuals. A mouse model was prepared that carries the single nucleotide change. A medium spiny neuron (MSN) model of HD was prepared, using an approach that permits the direct conversion of fibroblasts to neurons. Distinguishing this model from iPSC-derived neurons is that the model demonstrates the aggregation of endogenous mHtt. Most importantly, aggregation is observed in fibroblasts collected from patients within the most common repeat range of 40 to 50 repeats, a sharp distinction from the mouse models. Using this model, it was found that the depletion of Alfy significantly increases aggregation. An Alfy overexpression model showed that significant expression is achieved in the brains with no negative consequences, but that the hAlfy overexpressed can rescue the perinatal lethality observed in the Alfy KO. Studies indicate that the perinatal lethality and migration defects are due to selective MA. DVL3 is degraded by MA, which regulates Wnt-B-catenin signaling (Kadir et al Plos Genet 2016). The core Atg genes and Alfy lead to a synthetic lethal phenotype. Alfy directly binds to the autophagy machinery (Filimoneko 2010), and it binds directly to p62 (Clausen, 2010). p62 may be the sequestering agent of cargo. Alfy interacts with p62 and through its ability to interact with Atg5-12:Atg16L the autophagosome builds around that aggregate.

Aggregation accelerates death in neither the human MSN model nor the BACHD, but instead leads to neuronal dysfunction. Therefore, rather than a causative element, it is a contributing element to disease. Delaying or controlling aggregation might retain function longer, and delay onset.

Alfy may increase the targeting of mHtt to the APs to ensure that that is the means through which Alfy is exerting its function. In addition, the 3×FLAG tag on the hAlfy allows use of pull-down based approaches to determine if an interaction is occurring.

### Aims

Although a common theme across adult onset neurodegenerative diseases, the pathogenic role of aggregated proteins remains unclear. For the incurable familial neurodegenerative disorder Huntington's disease (HD), resolving the accumulation of mutant huntingtin (mHtt) is highly correlated with favorable therapeutic outcomes. Nonetheless, since all species of mHtt is eliminated, whether aggregate clearance per se is beneficial is unknown.

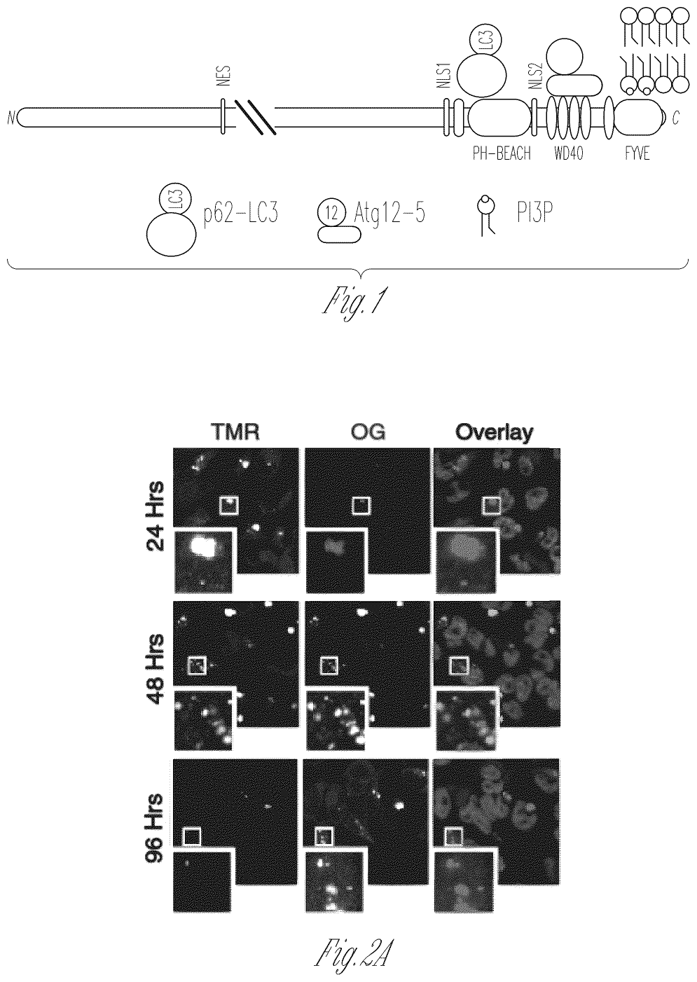

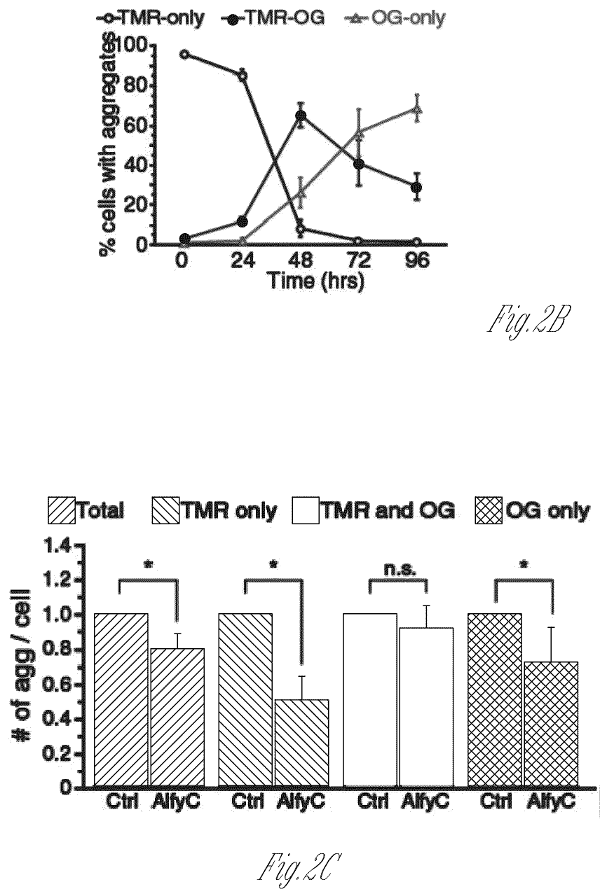

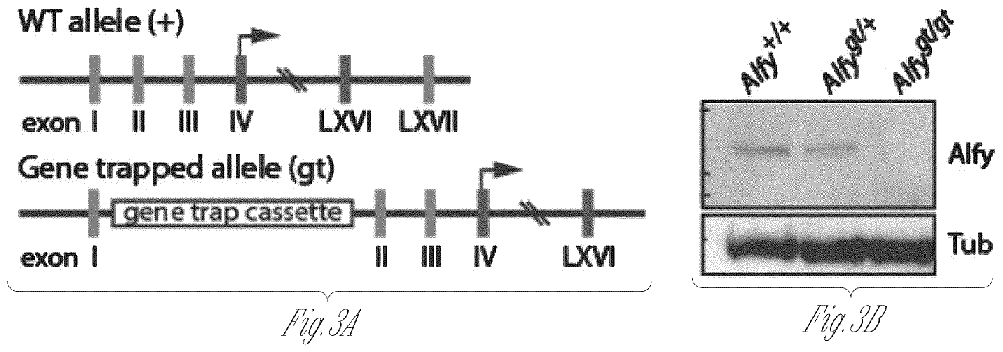

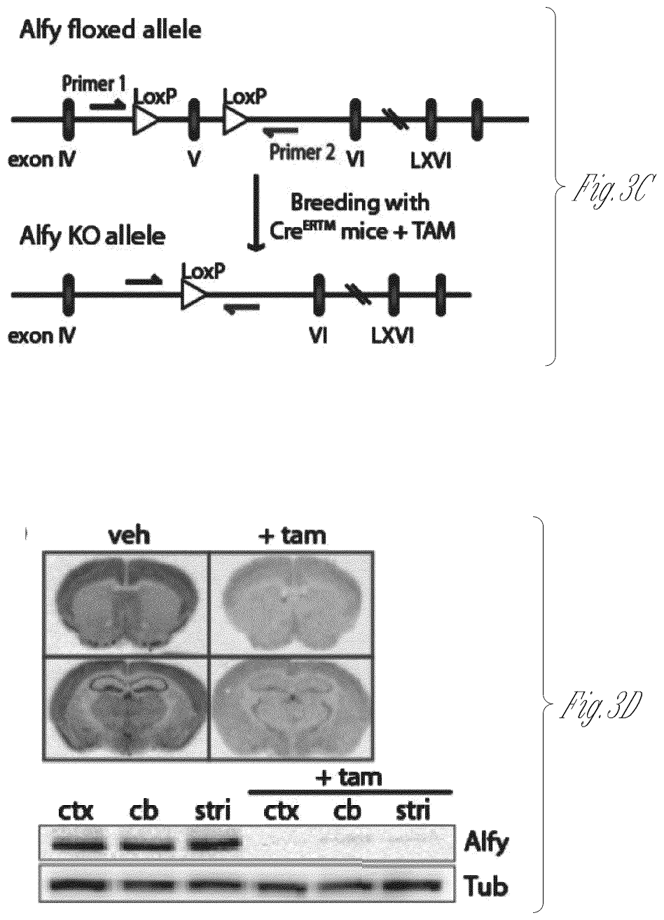

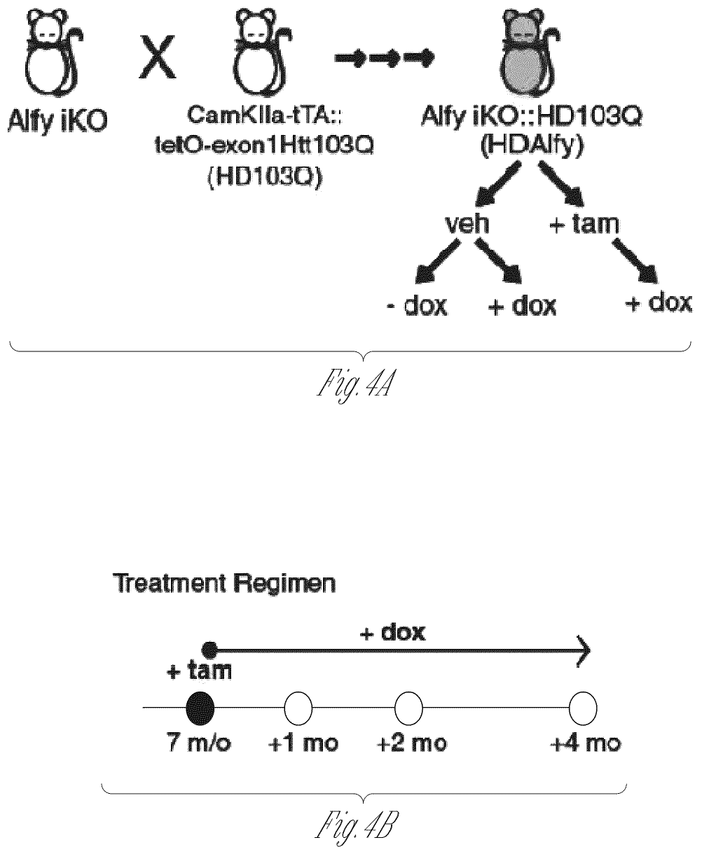

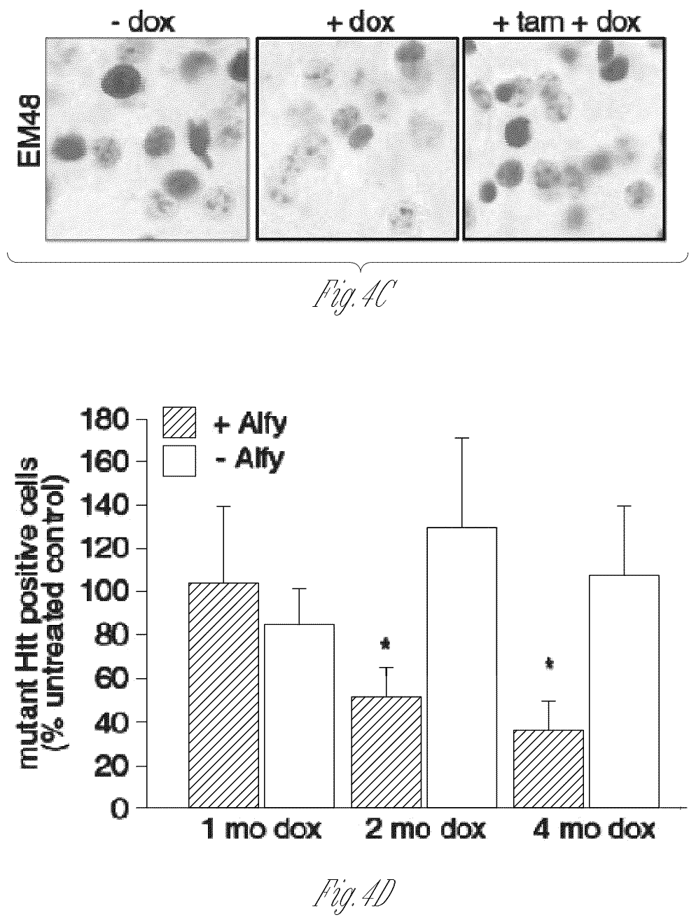

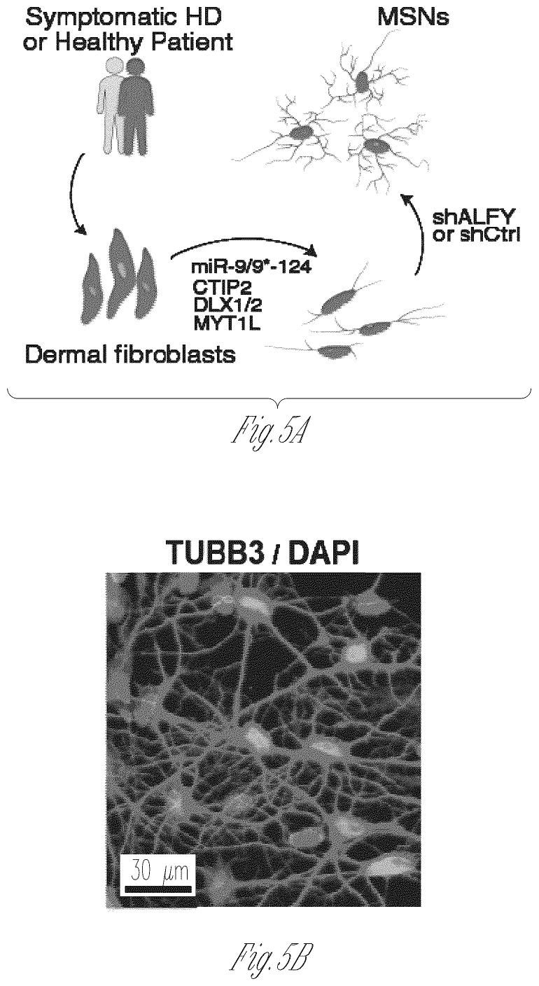

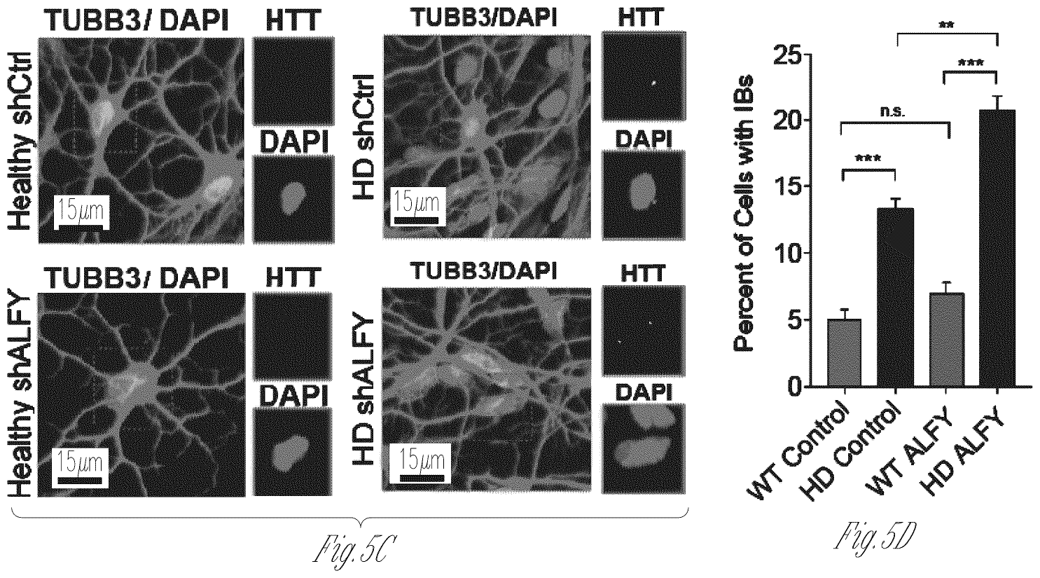

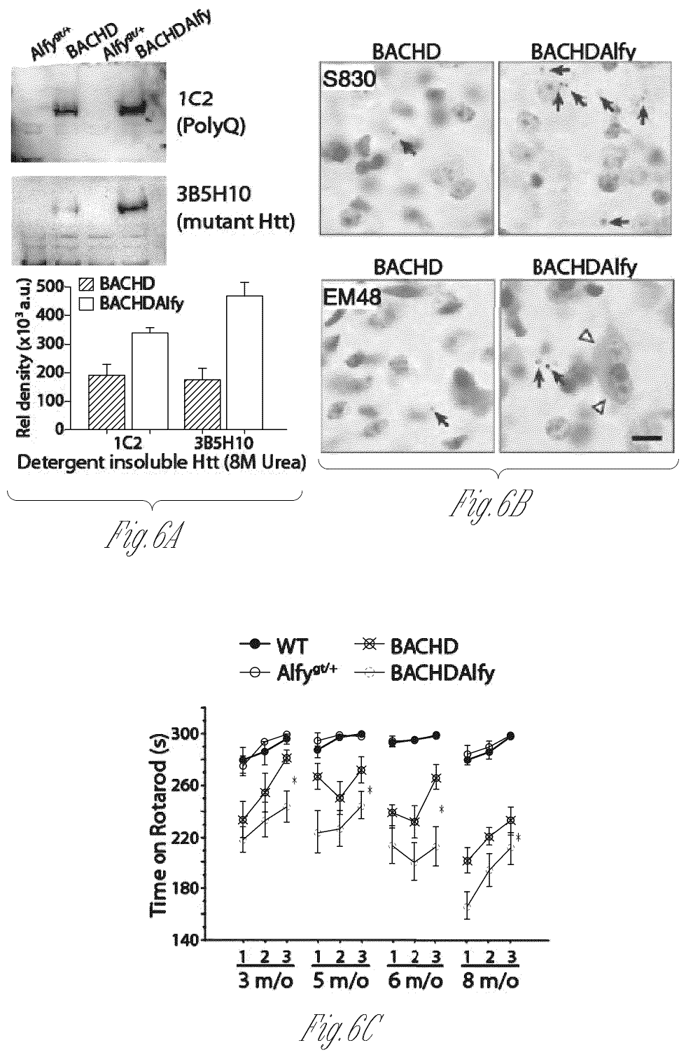

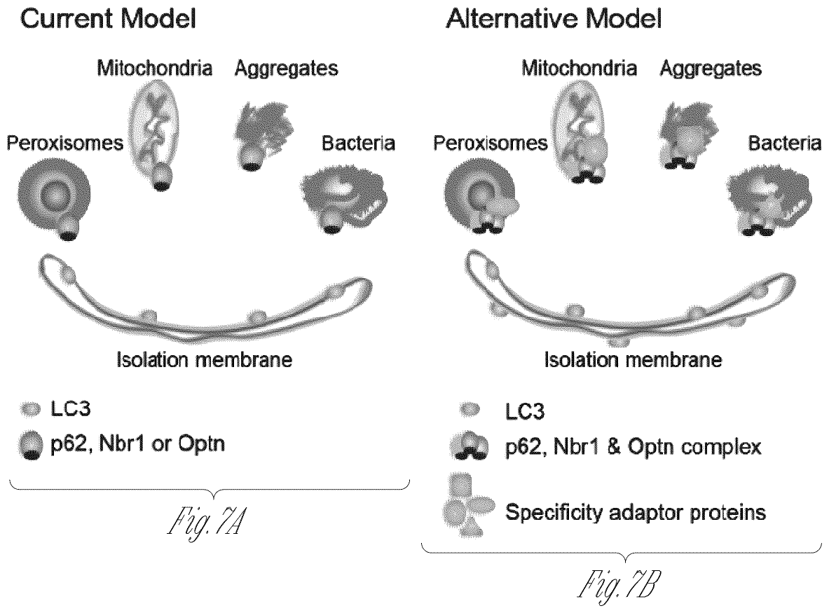

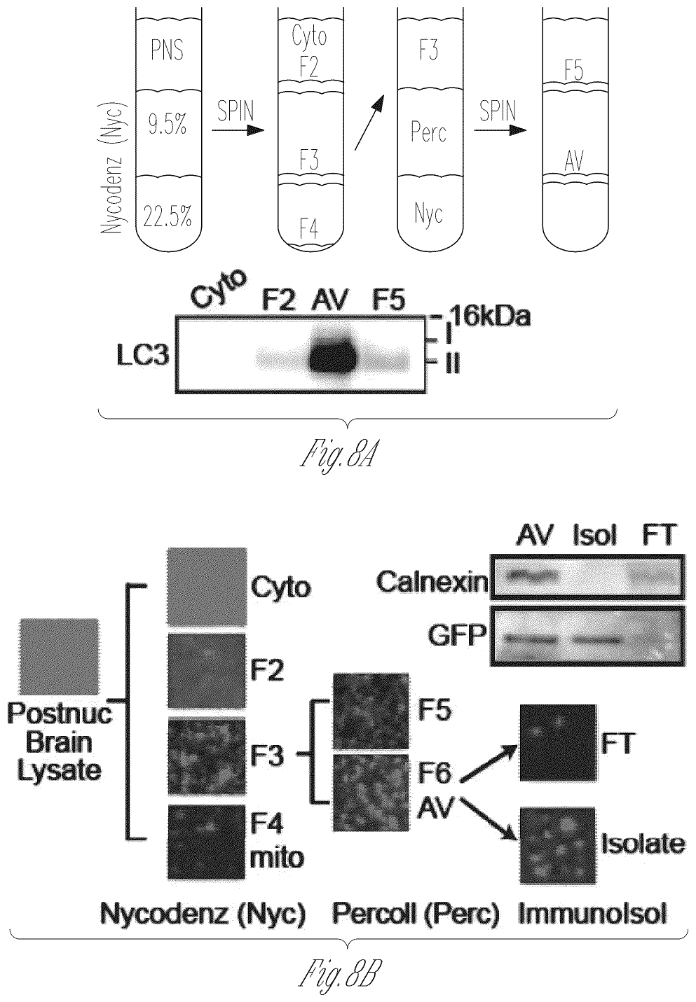

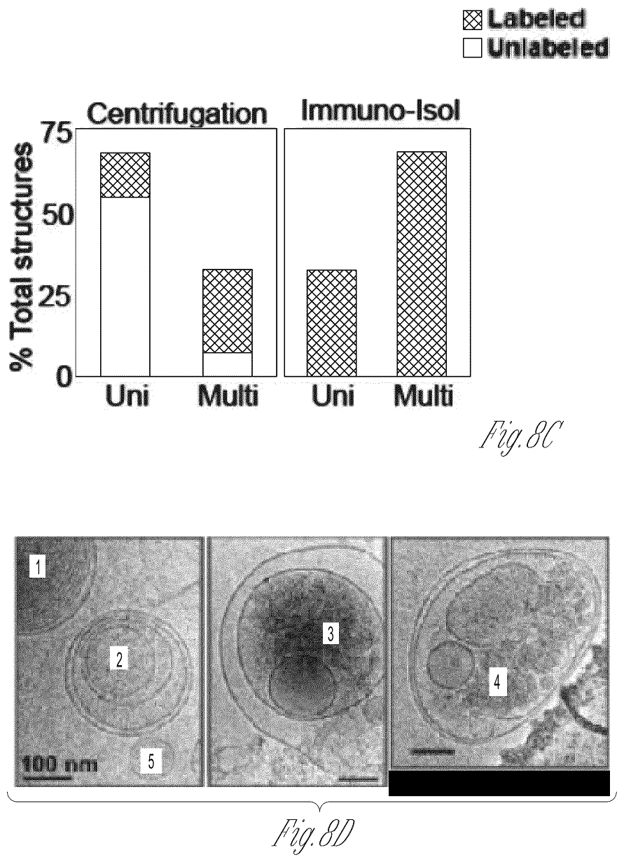

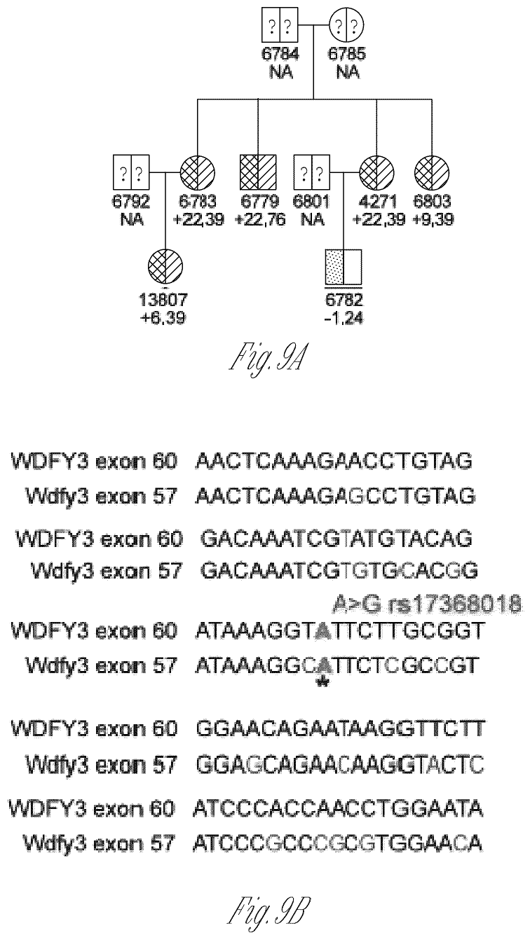

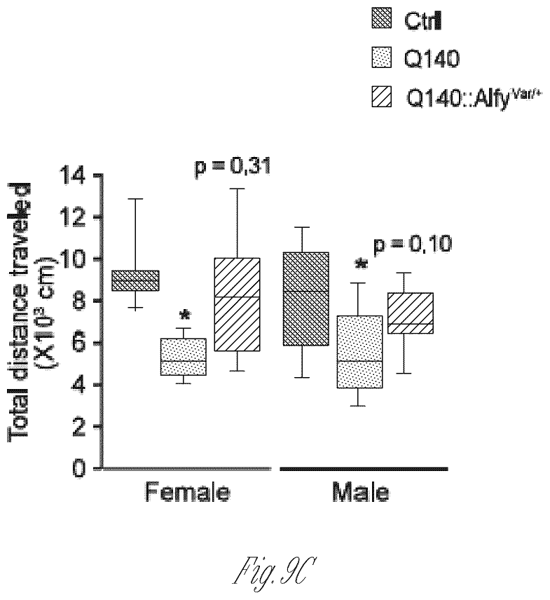

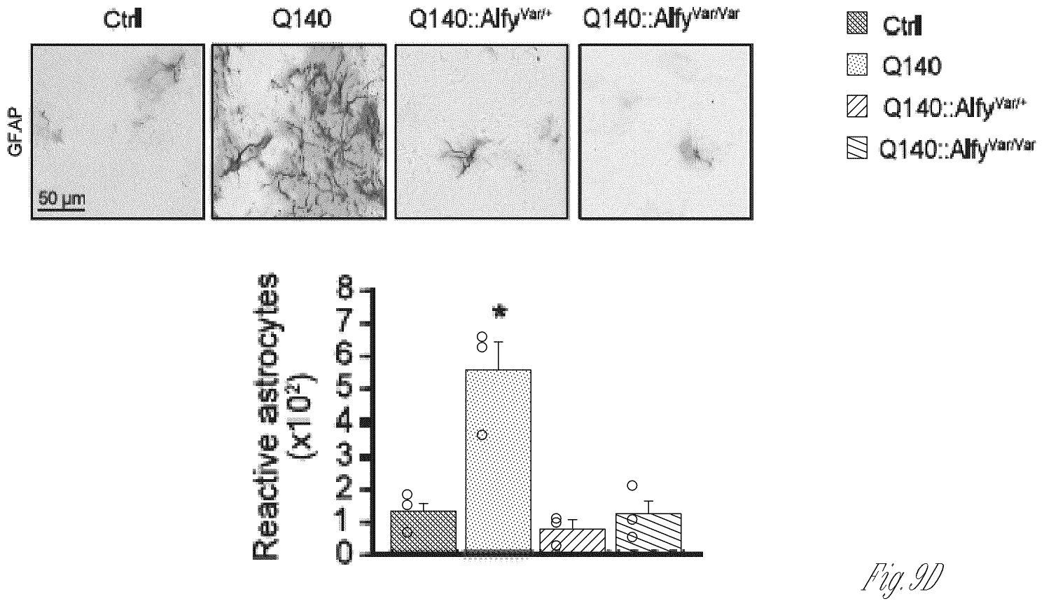

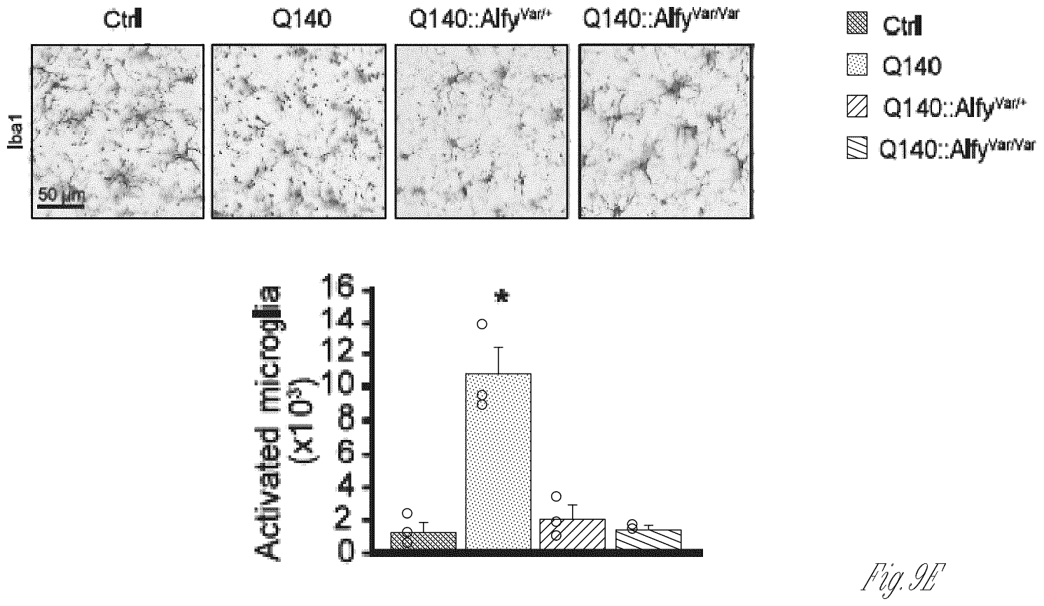

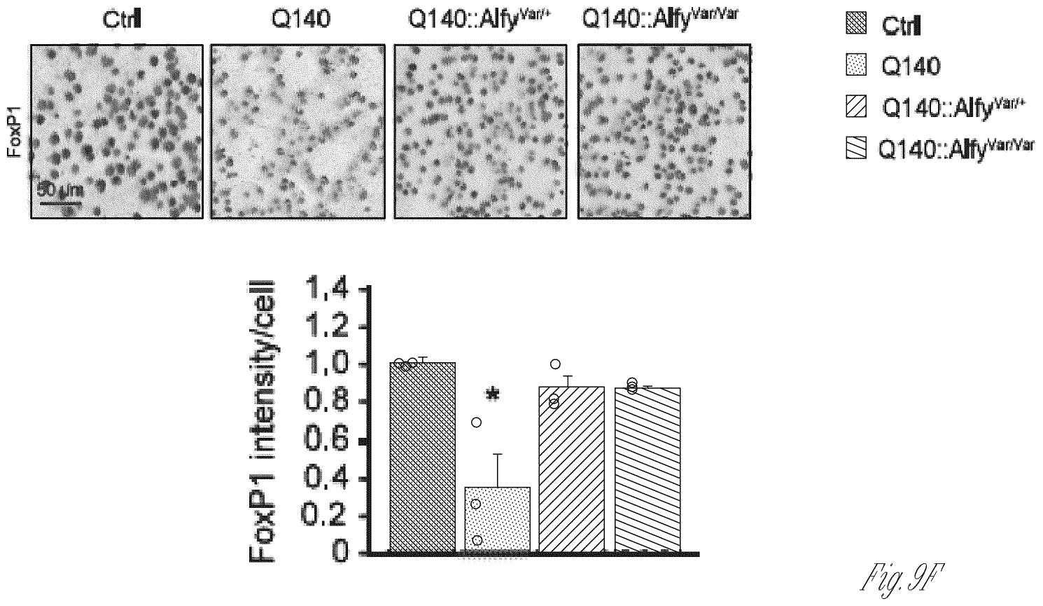

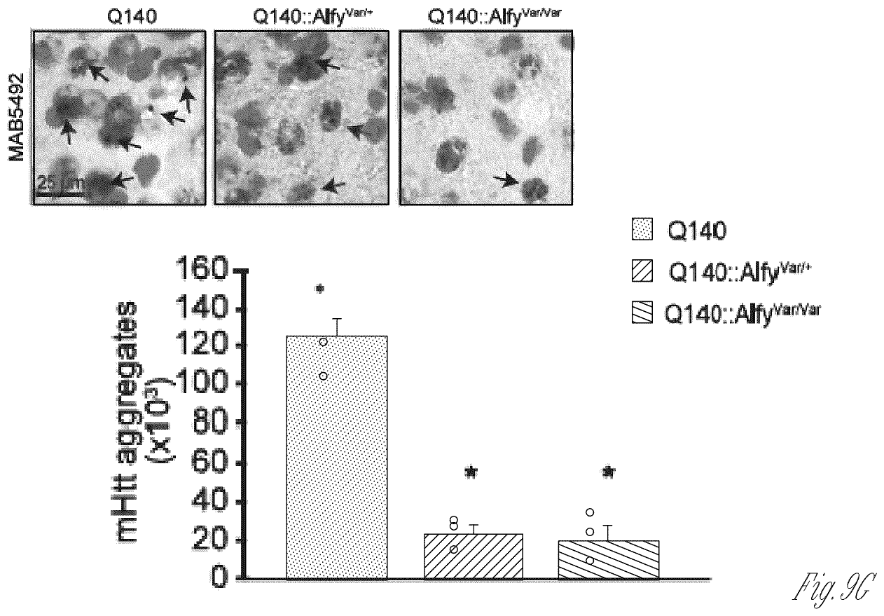

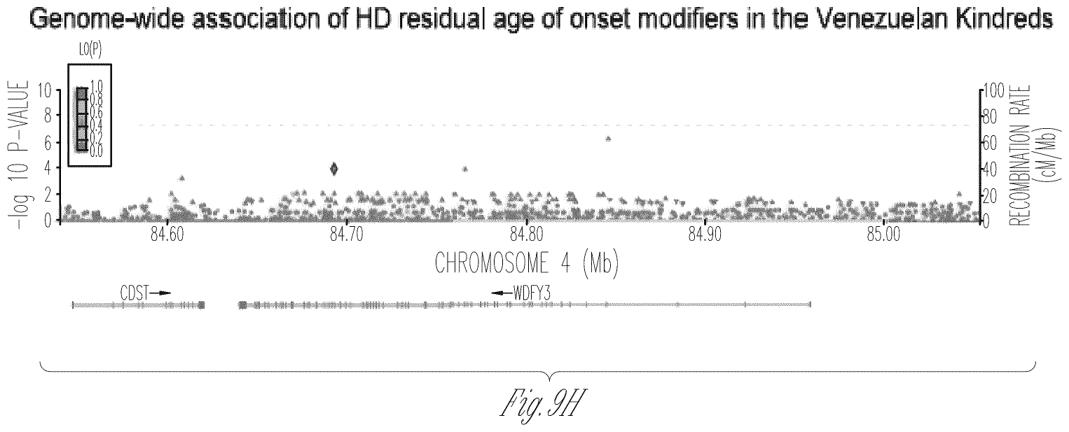

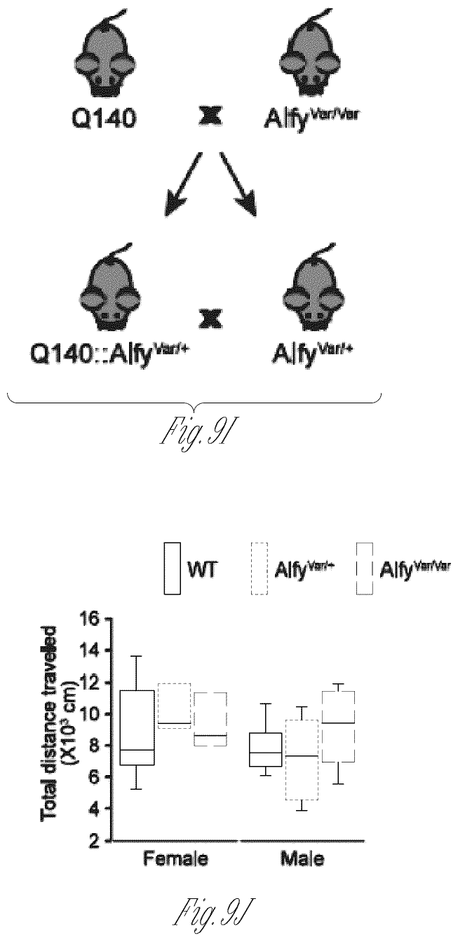

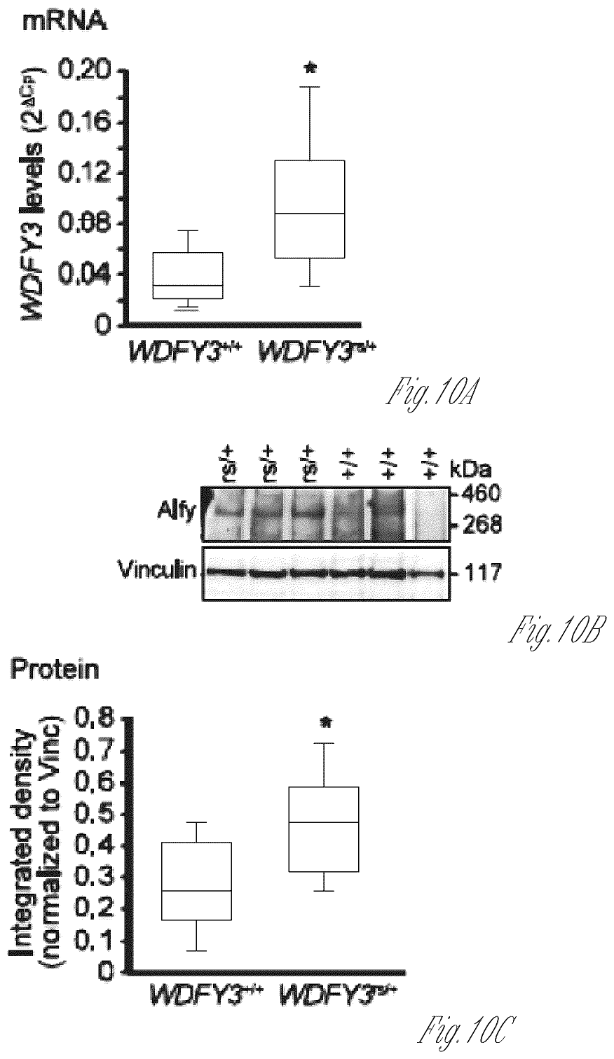

To study the possible contribution of aggregated mHtt to pathogenesis, it is determined not only that macroautophagy (MA) is the means through which this is achieved, but that the Autophagy linked FYVE protein (Alfy) acts as a molecular scaffold between aggregated mutant Htt and the MA machinery (FIG. 1). The cell-based work revealed that Alfy is required for aggregate-clearance. These findings put forward the potential that by modulating Alfy levels, aggregate-clearance in the adult brain may be sufficient to impact HD pathogenesis.

In cell models, Alfy eliminates preformed inclusions, and that augmenting Alfy levels increases the rate of aggregate clearance. Moreover, Alfy is a brain-enriched protein that is essential for life, and is essential for the turnover of aggregated proteins in the adult brain. In addition, it was found that diminishing Alfy levels in patient-derived neurons augmented the accumulation of endogenous mutant Htt aggregates, and in vivo, leads to an accelerated appearance of several disease-associated phenotypes, including behavioral deficits and neuropathological indicators of neuronal stress. Consistent with the findings implying Alfy might be a modifier of HD, a study of genetic modifiers from the Venezuelan Cohort revealed that a variant in Alfy can significantly modifier the age- on-onset in patients. Interestingly, the variant is a single residue change within the coding region of Alfy that is associated with a delayed age-of-onset for an average of ten years.

### Determine if Augmenting Alfy Levels In Vivo Modifies Phenotypic Onset in Mouse Models of HD.

In stable cells, increasing Alfy levels can augment the clearance of preformed inclusions. Alfy levels are increased in vivo to test the hypothesis that increasing Alfy levels delay onset of HD in mice.

Determine the Impact of Augmenting Alfy Levels In Vivo after Symptomatic Onset.

The clearance of aggregates has been associated with alleviating symptoms in mouse models of HD. To test the hypothesis that aggregate-clearance is sufficient for therapeutic benefit, Alfy expression is induced in adult, symptomatic mice.

Determine the mechanism of action of the disease modifying variant Alfy(13032V).

Using model systems, the hypothesis that the variant modifies HD via altering aggregate-clearance kinetics is tested.

Establish the molecular mechanism underlying Alfy-mediated selective MA of aggregates.

Although Alfy is required for the selective degradation of aggregated proteins by MA, other autophagy adaptor proteins have been implicated in playing a role as well. In a competing model regarding selective autophagy is employed, using the cell-based model of HD aggregate-clearance.

### Significance

Abnormal accumulation of aggregated proteins is a hallmark across the majority of adult onset neurodegenerative diseases including Huntington's disease (HD). HD is a rare hereditary disorder with a frequency of 5 to 10 cases per every 100,000 individuals, affecting approximately 70 million patients of European descent (Walker, 2007). HD is caused by a CAG trinucleotide repeat expansion in exon1 of the HD gene. Its translation leads to the appearance of proteinaceous deposits, which is comprised, at least in part, with a mutant huntingtin (mHtt) protein product with an expanded polyglutamine (polyQ) tract. A devastating disorder in its own right, the genetics of HD, both in its inheritance pattern (autosomal dominant, near complete penetrance) and the age-of-onset predictive values of the causative mutation (MacDonald & Gusella, 1996), makes HD a paradigm disorder by which we may understand how protein aggregation might impact more frequently occurring, sporadic diseases.

Elimination of mHtt expression in mouse models of HD using various approaches (Yamamoto et al., 2000, Harper et al., 2005; Machida et al., 2006; DiFiglia et al., 2007; Snyder-Keller et al., 2010; Southwell et al., 2009; Wang et al., 2008) have consistently revealed that the insoluble aggregated proteins, despite heterogeneity in structure and cellular distribution, can be eliminated by neurons in the adult brain.

The lysosomal degradation pathway macroautophagy (MA) has been implicated by several groups to degrade protein aggregates (Yamamoto & Yue, 20014). MA captures its cargo in a transient organelle known as the autophagosome (AP), which is formed by a hierarchical assembly process governed by a series of autophagy (Atg) proteins (Itakura & Mizushima, 2010). Using ImmunoEM or subcellular organelle fractionation, it was found that aggregated proteins less than or equal to 1 micron in diameter are selective captured into the AP through a process known as selective MA, which relies on the adaptor protein Alfy (Filimonenko et al., 2010). Loss of Alfy inhibits the selective clearance of aggregated Htt, but not the turnover of cargoes during basal or starvation-induced degradation.

### Overview

Protein aggregation and their role in neurodegeneration has been a long-standing topic of interest in neurodegenerative disease research, but it is still uncertain whether the elimination of protein accumulation can be beneficial. The limitation confronting these studies has been the inability to study the turnover of aggregated proteins without grossly affecting degradation overall. Furthermore, although protein degradation pathways are readily hindered, their activation has been difficult to achieve. To understand how selective autophagy, plays a role in aggregate-clearance, with an emphasis in the adult brain and second, the relative importance of protein aggregation in disease pathogenesis is determined. Although there is a focus on HD pathogenesis, given the importance of protein accumulation across neurodegenerative diseases, including the accumulation of products due to RAN translation, as well as the importance of selective autophagy across neurodegenerative diseases, the findings give insight across a wide array of neurodegenerative diseases. Tools are employed that allow for the spatial and temporal dissection of the role of Alfy in aggregate clearance, e.g., using the cell line created to study aggregate clearance in the constitutive presence of aggregation-prone protein, as well as the use of the medium spiny neuron model derived from symptomatic HD patients.

### Results

Using a newly created cell line that allows temporally labelling discrete pools of protein (FIG. 2), and therefore temporally segregate analyses of the aggregates, it was determined that at any given time there are at least 3 pools of aggregates, a preformed pool, a newly formed pool and a ‘growing’ pool, the latter of which represents aggregates that are continuously incorporating protein (Eenjes et al., 2016). Increasing levels of Alfy can decrease the total amount of aggregates, specifically by impacting the pool of preformed inclusions (Gray et al., 2016).

Next, it was determined that Alfy is necessary for the clearance of aggregated proteins in adult brain (FIG. 4). To do so, several mice were prepared that allowed for genetically modifying Alfy levels (FIG. 4), including an Alfy inducible KO model (iKO) (created by crossing Actin^{CreERTM/+} to Alfy^{loxP/loxP} mice (1)), which was crossed to a newly created tet-regulatable HD model (HD103Q, CamKIIa^{tTA/+}::TetO^{Ex1Htt103Q/Ex1Htt103Q}) to merge the six genetic modifications necessary to create Alfy iKO:HD103Q mice (FIG. 4). This model allows for the adult-inducible KO of Alfy (upon administration of tamoxifen (tam)) in an aggregate-clearance paradigm in the adult brain. Similar to cell based studies, this model demonstrated that Alfy is necessary for efficient turnover of aggregated proteins.

Given the function of Alfy, it was established if altering Alfy levels could modify HD pathogenesis (FIG. 5). A direct conversion based approach is used to convert symptomatic HD patient-derived fibroblasts to a human neuronal model of HD (Abernathy et al., 2017; Abernathy & Yoo, 2015; Victor et al., 2014; Victor et al., 2018; Richner et al., 2015). In contrast to iPSC-derived neurons, direct conversion of fibroblasts maintains the age signature of patients when the fibroblasts were collected, and practically, this contributes to a feature of this model which is the accumulation and aggregation of endogenous mHtt. Depletion of Alfy in this human neuronal model leads to an increased accumulation of endogenous mHtt (FIG. 5). Depletion of Alfy (Dragich et al., 2016) in the BACHD model similarly leads to an increased accumulation of detergent insoluble mutant Htt. This accumulation correlates with an accelerated onset of behavioral dysfunction (FIG. 6). Interestingly, in neither model did increased aggregation accelerate the appearance of cell death, suggesting that aggregation might influence neuronal function, but not toxicity.

In a GWAS study identifying potential genetic modifiers of age-of-onset in HD, Wexler, Housman and colleagues identified a peak at 4q21, the chromosome localization of Alfy. Follow-up studies by the Housman lab found that the variant that most statistically correlated with the modifier effect was a coding variant of Wdfy3, the gene that encodes for Alfy. Interestingly, the rare variant Alfy(13032V) (1% predicted frequency) identified was a single base pair change within the coding region of Wdfy3, which was associated to a delayed age of onset by almost ten years. Immunoblotting analyses were performed for levels of Alfy in patient brains. There is statistically significant higher levels of Alfy in Alfy(13032V) brains. Given the delayed age of onset exhibited in patients with this variant, these findings strengthen the hypothesis that increased Alfy levels may be beneficial.

### Augmenting Alfy Levels In Vivo and Phenotypic Onset and Progression in HD

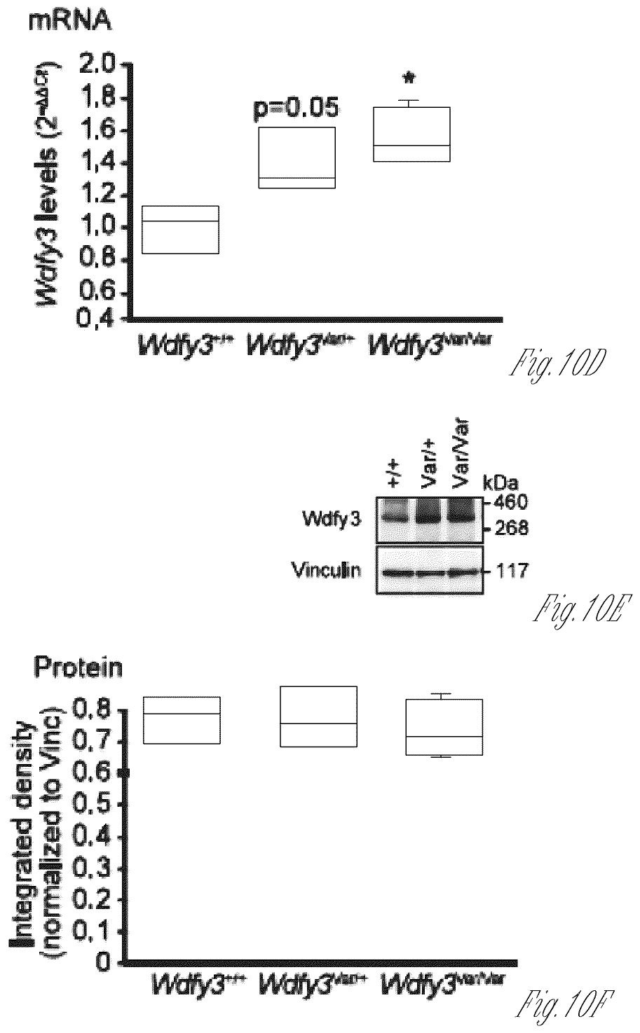

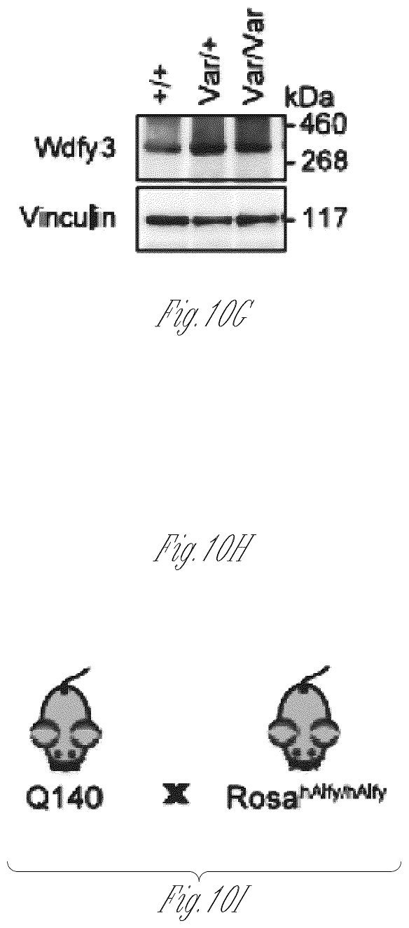

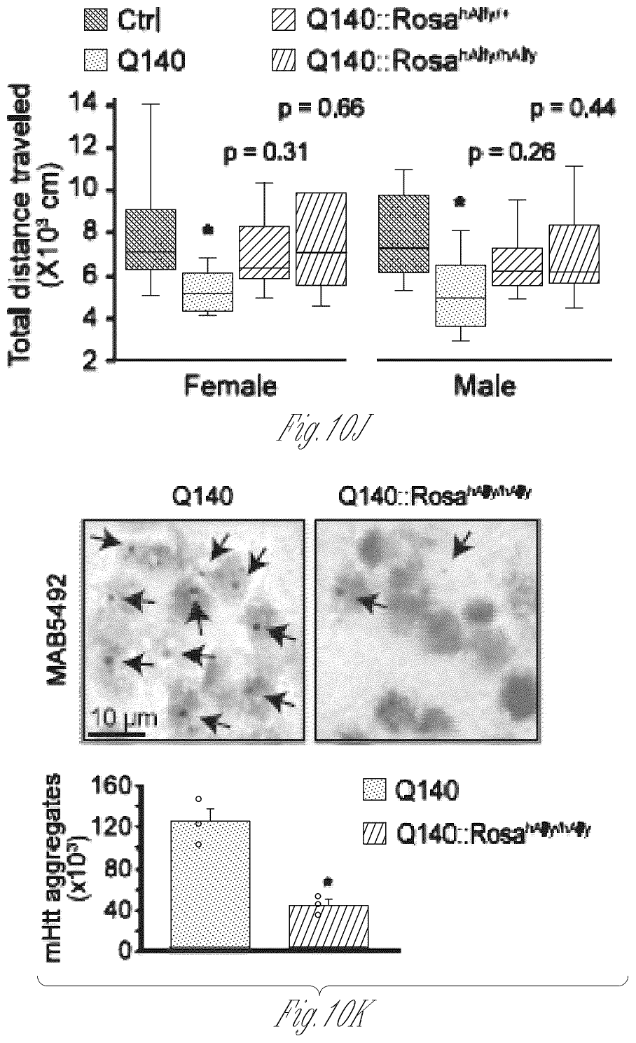

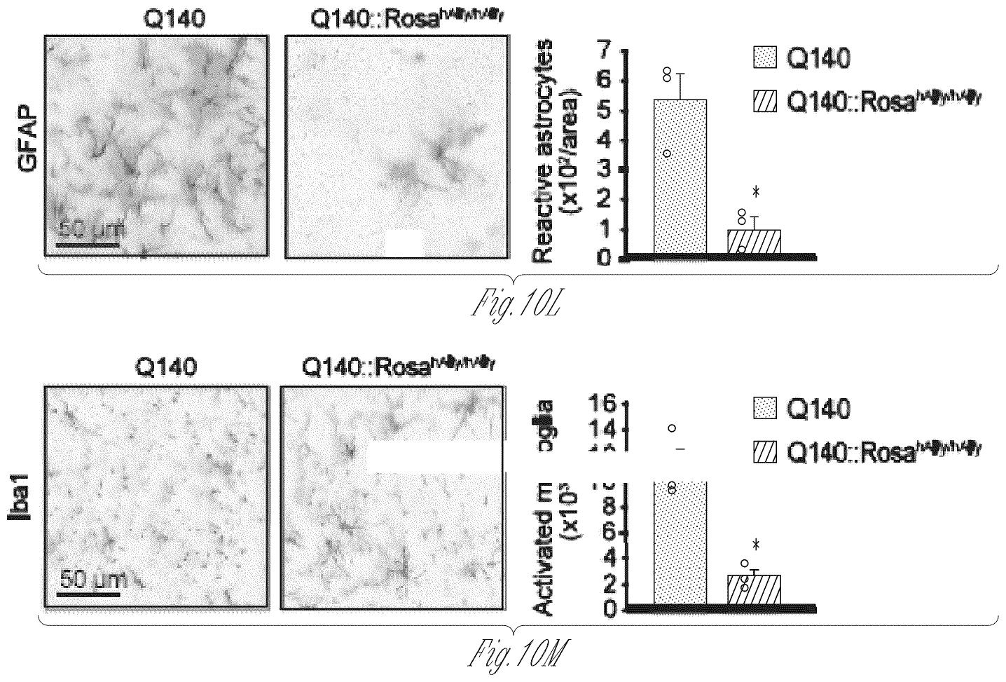

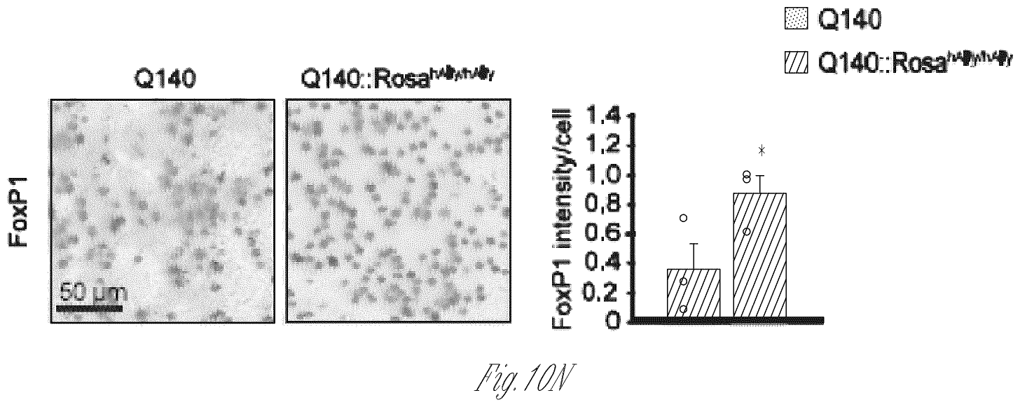

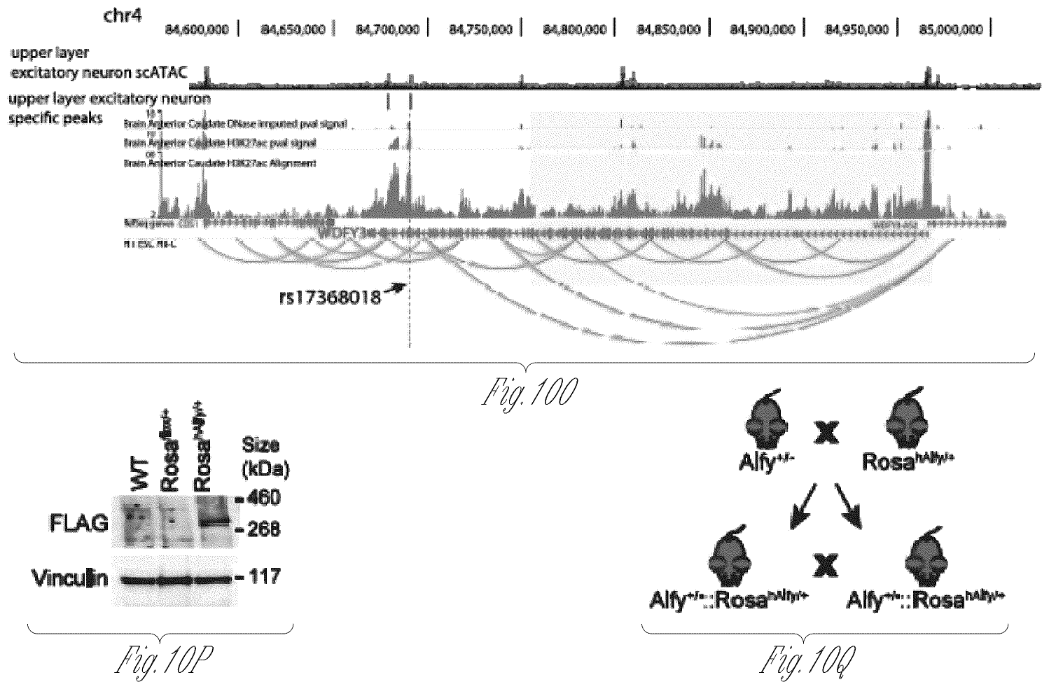

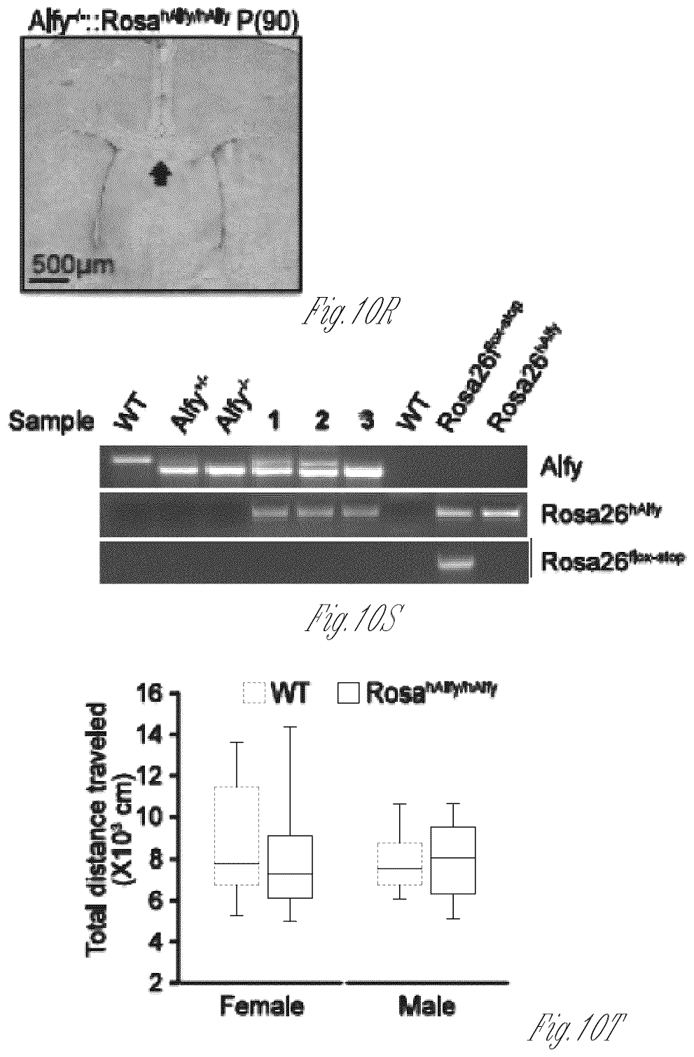

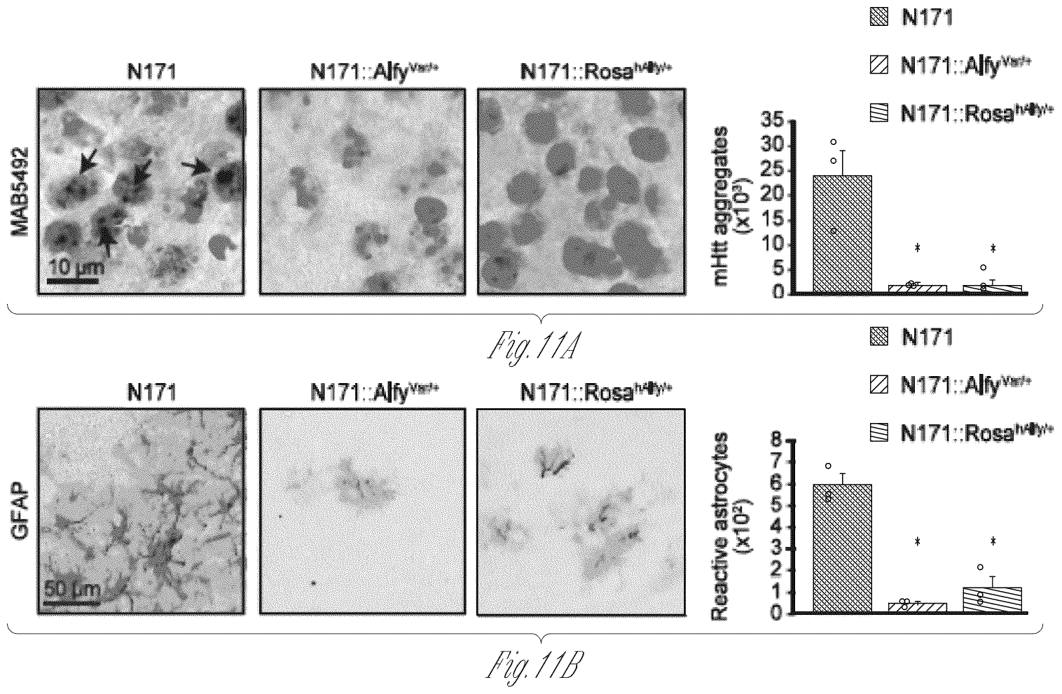

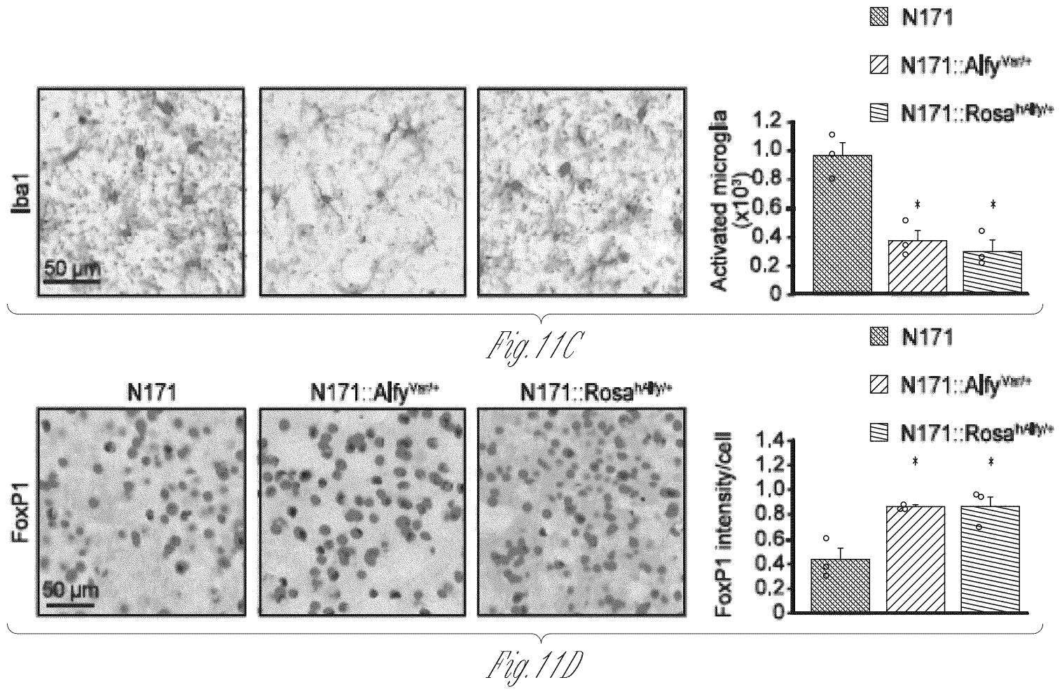

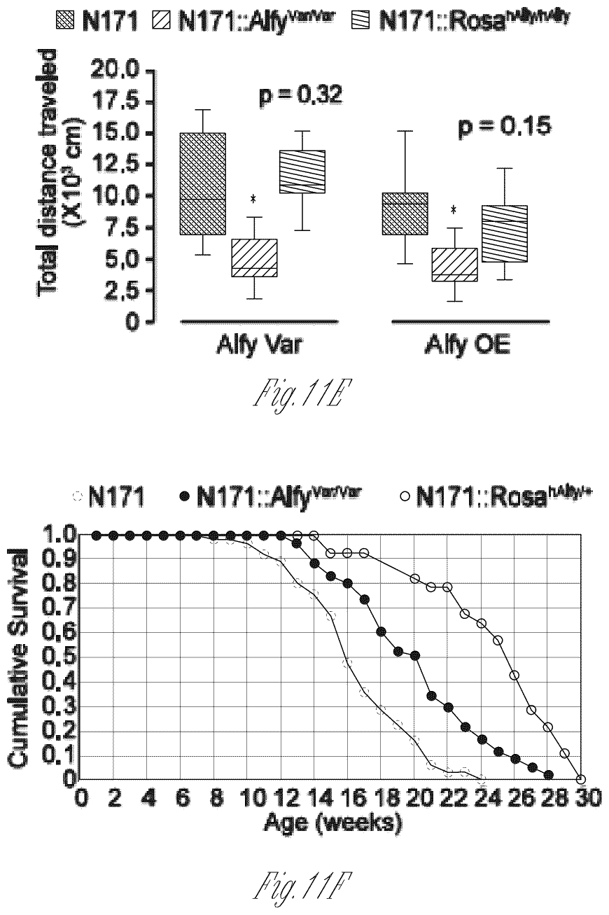

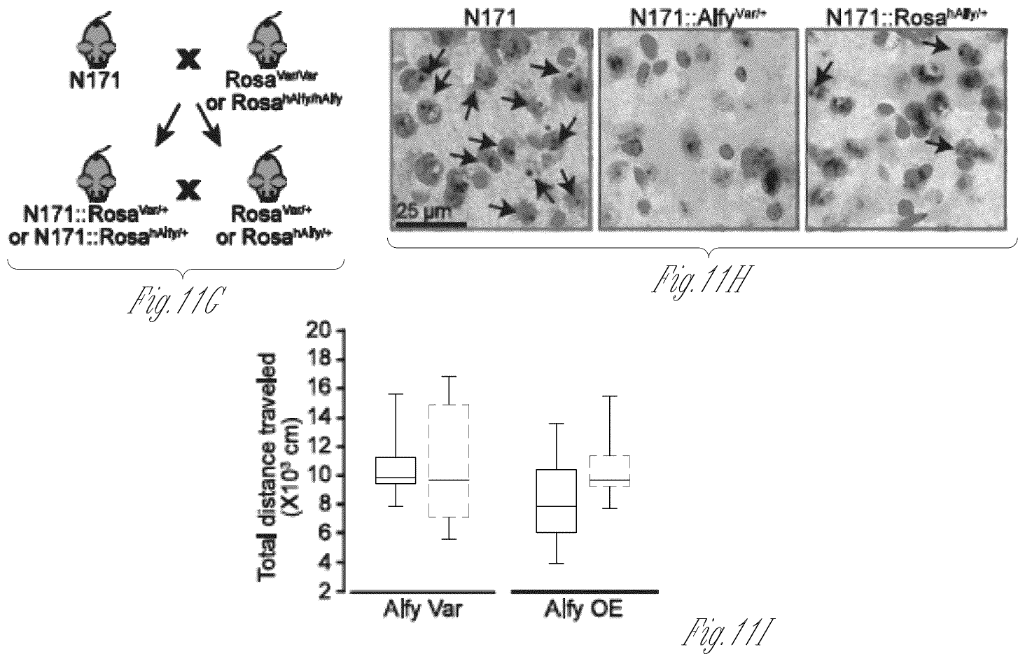

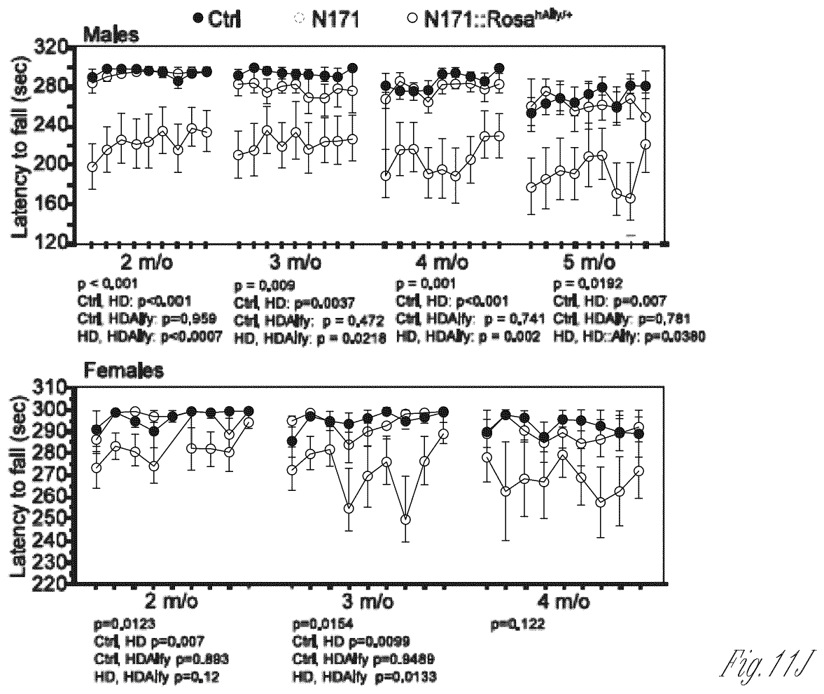

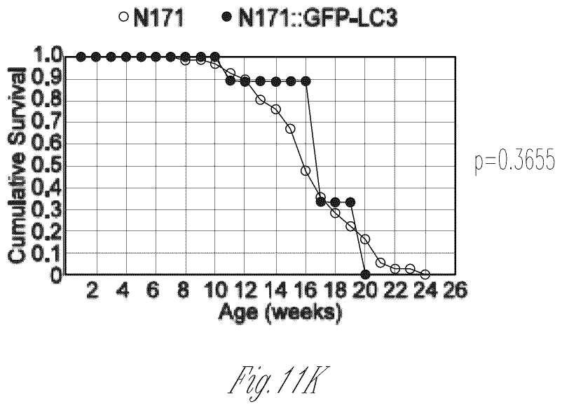

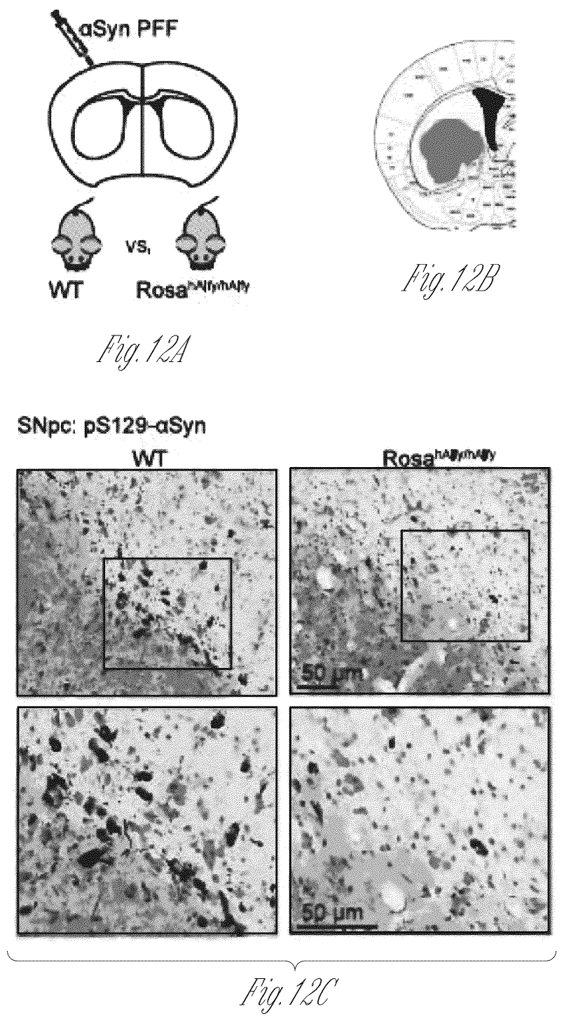

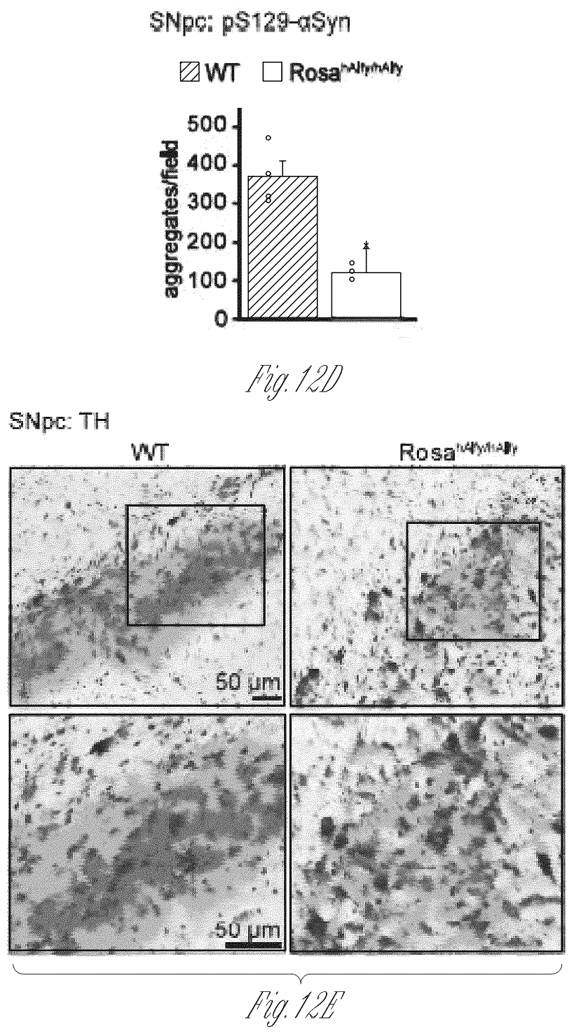

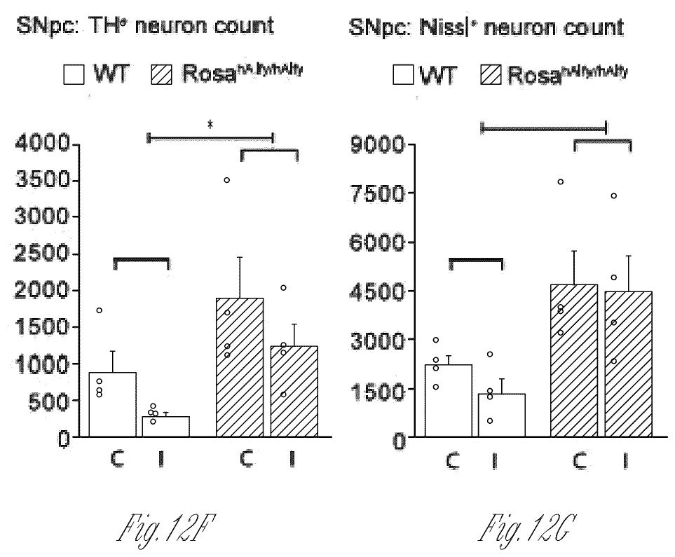

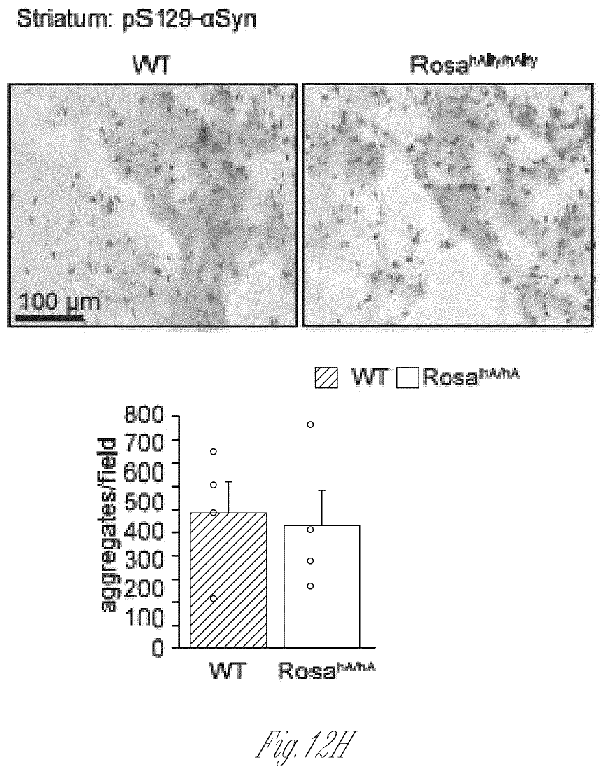

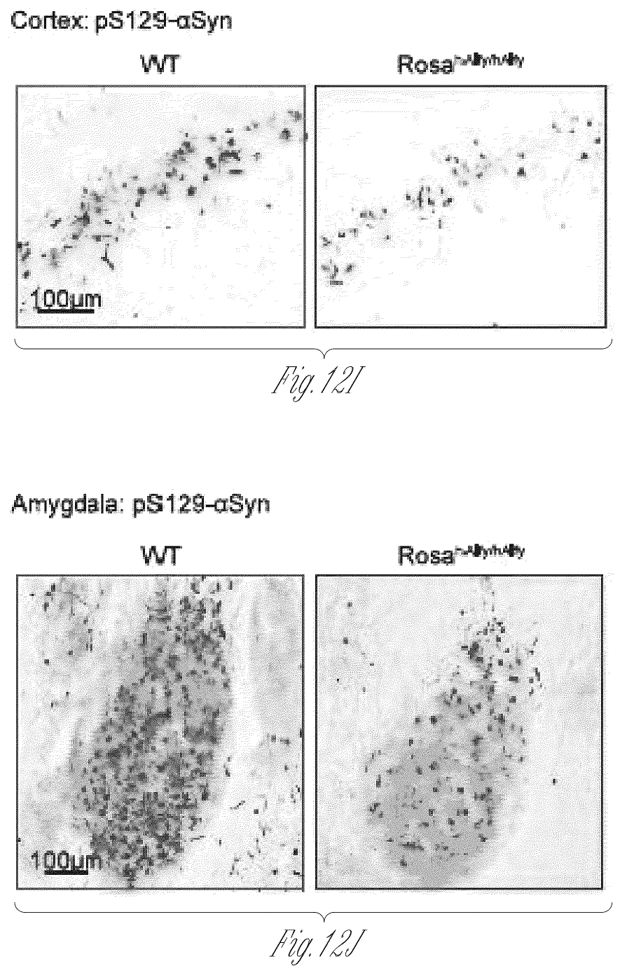

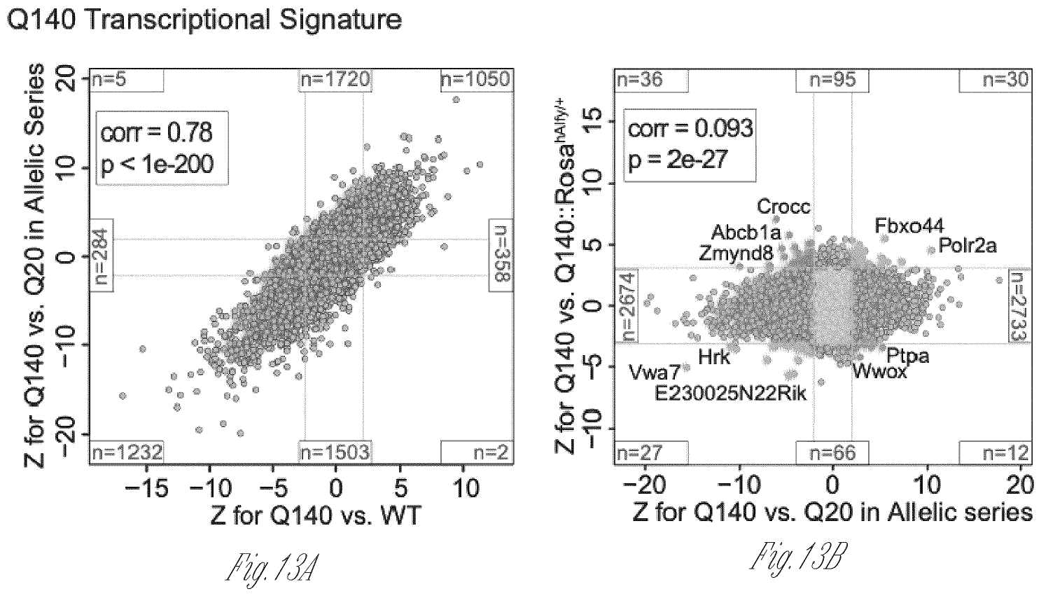

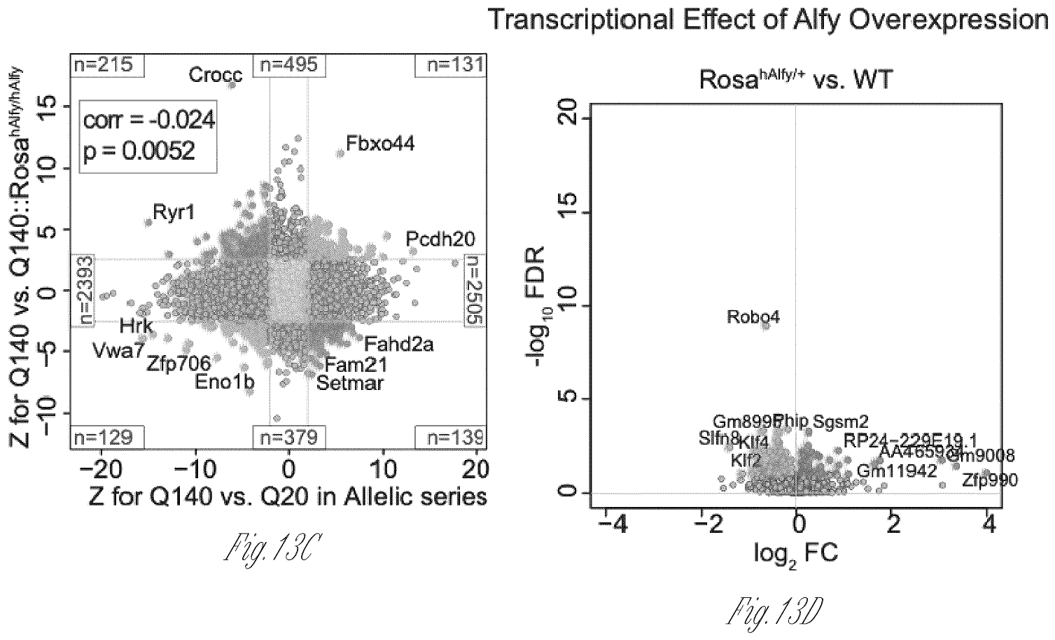

Given cell-based studies indicating that augmenting Alfy levels enhances aggregate clearance (FIG. 2), and the impact of the loss-of-function studies (FIGS. 4-6), a mouse model was created in which Alfy levels can be conditionally augmented (FIG. 10). 3xFLAG-human Alfy (hAlfy) cDNA was knocked into Rosa26 locus preceded by a flox-stop cassette. Cre-excision permits hAlfy expression by the endogenous Rosa26 promoter. To examine expression and confirm functionality of the introduced Alfy construct, mice were crossed to Hprt^{Cre/x+} mice to delete the flox-stop cassette in a heritable manner, thereby creating a constitutive overexpression line (Rosa26^{hAlfy/hAlfy}). Importantly, 3xFLAG-hAlfy can functionally replace the loss of endogenous Alfy:Alfy KO mice, which are normally perinatal lethal, are still viable at P21 in the presence of hAlfy. The oldest AlfyKO::Rosa26^{hAlfy/hAlfy} mice are currently 8 m/o and appear normal and can breed. In this aim, we will use this constitutive overexpression model to determine if the onset of neuropathology and behavioral symptoms of two HD models will be delayed with increased doses of Alfy.

### Overexpress Alfy in Mouse Models of HD

Three models of HD are employed: The N171-82Q model (N171) (Schilling et al., 1999), BACHD (Gray et al., 2008) and zQ175 knock-in model, as homozygous (zQ175^{KI/KI}) (Menalled et al., 2012). These models were selected to balance strengths and weaknesses such as Htt length, CAG repeat design and phenotype severity. These models are crossed with Rosa26^{hAlfy/hAlfy}.

zQ175 mice are crossed to create zQ175^{KI/KI}::Rosa26^{hAlfy/+} mice. Intercrosses of these lines create the littermate controls for all of the experiments. In the meanwhile, N171 mice are crossed similarly, however, given the limited breeding time and lifespan of the mice, as well as the transgenic nature of the mice, N171+/−::Rosa26^{hAlfy/+} mice are intercrossed to give the necessary animals. Since Alfy overexpression has not been fully characterized, the N171-negative mice resulting from this cross are also tested to examine the impact of hAlfy overexpression in the absence of a disease background.

A mouse model was created. The Alfy over-expression mice appear normal at 8 m/o. A different Cre driver such as Nestin^{Cre/+}, or limiting forebrain excision with CamKIIa^{Cre/+} could be employed

Increasing Alfy Levels and mHtt Accumulation

Several studies implicate MA in the clearance of aggregated polyQ proteins (Iwata et al., 2005a; Iwata et al., 2005b, Ravikumar et al., 2005; Ravikumar et al., 2002; Yamamoto et al., 2006), consistent with our preliminary findings (FIGS. 4-6). As a follow-up to cell-based data (FIG. 2) it is determined if Alfy overexpression in vivo leads to the contrary.

IHC (n=8 brains/genotype/sex/age): Mice are transcardially perfused with 4% paraformaldehyde. Dissected brains are weighed, cryoprotected in 30% sucrose, then sectioned to 30 μm sections. Sections at 240 μm increments are stained for mHtt using EM48, S830 and MW8. Sections are co-stained for Nissl, then assessed stereologically for aggregates/cell. Nuclear and cytoplasmic aggregates are scored separately. As CAG repeat expansion mutations have also been implicated to induce repeat-associated non-ATG (RAN) translation (Banez-Coronel et al., 2015), thus the presence of aggregates containing RAN proteins (using a-polyA-Ct, a-poly-S-Ct, a-polyL-Ct, and a-polyC-Ct antibodies) are probed as described (Banez-Coronel et al., 2015). HD mice are compared to HD littermates that are positive for Rosa2^{hAlfy/+} or Rosa26^{hAlfy/hAlfy}.

The time course for each mouse model is established based on already published findings regarding when aggregates appear. N171-82Q^{+/-} mice demonstrate ubiquitin-positive Htt inclusions by 3-4 m/o (Yu et al., 2003; Ferrante, 2009), thus brains are collected monthly between 2 and 6 m/o, and at end-stage as appropriate. In zQ175KI/KI mice, diffuse nuclear staining, nuclear micro-aggregates, and neuropil aggregates are observed at 1 to 2 m/o in the striatum nuclear inclusions are then additionally expressed by 4 m/o (Menalled et al., 2003), thus, brains are collected at 2, 6, 10, and 14-month time points for this model. BACHD mice have a very late aggregation phenotype, beginning at 12 m/o (FIG. 6) (Gray et al., 2008) and thus brains are collected at 9, 12, and 15-months.

Biochemistry (n=5 brains/genotype/sex/age): Brain lysates at the ages listed are also collected to determine the status of SDS-soluble and -insoluble mHtt. This is performed by examining total homogenates from dissected brain regions (cortex, striata, hippocampus and cerebellum). Total lysates (modified RIPA buffer with 1% TritonX-100, 1% NP-40, protease and phosphatase inhibitors, NEM (to inhibit deubiquitinating enzymes)) are pelleted at 14K rpm. The resulting pellet is resuspended in modified RIPA+8M urea to solubilize will be examined at the ages listed for IHC by western blotting (anti 1C2, MAB2166 1:1000 anti-Ub, Stressgen, 1:3000) or by filter trap analysis (Eenjes et al., 2016; Filimonenko et al., 2010).

### Increasing Alfy and the HD-Like Phenotype.

Mice are processed for neuropathology, and placed through a lab-standardized behavior battery to determine if symptomatic onset is delayed upon Alfy overexpression (cohort1), and if a correlation between aggregate load and symptomatic outcome is present (cohort2). HD-mice are also assessed.

Neuropathology (n=8/genotype/sex/age): Sections are immunostained for GFAP and Iba1 or GFAP and NeuN. Stereological analyses will be performed for area (striatum, cortical regions and hypothalamus) and neuronal counts (NeuN). GFAP and Iba1 are initially examined qualitatively, and should experiments warrant, similar stereologic approaches are used to quantify the events. Analyses are conducted at the same time points during which aggregation is assessed. Volumetric changes and neuron loss are quantified stereologically. Neuron loss is assessed as total (via Nissl) or enkephalin+ neurons, the latter of which represents the neurons first affected in HD.

Behavior Battery: (cohort1, n=12; cohort2, n=8 per genotype per sex): For the behavioral assessment, 2 cohorts of animals are used. Cohort 1 comprises of a traditional longitudinal study that will repeatedly examine the cohort. Cohort 1 continues to be analyzed until mice overexpressing hAlfy demonstrate significant behavioral onset, for every month (N171-82Q^{+/-}), 3 months (BACHD), or 4 months (zQ175^{KI/KI}) then sacrificed for neuropathology. All models with their respective littermate controls are assessed starting at age 2 m/o. The N171-82Q^{+/-} mice show diminished survival, with significant loss of life starting at 6 m/o. Survival is therefore monitored every 2 weeks. Cohort 2 is tested immediately prior to being sacrificed. The following behavioral battery is assessed during the dark phase of the diurnal cycle:
1. Open Field (day 1): Mice are habituated for 60 minutes to the maze upon the first exposure. Test is conducted every month for 30 minutes, and measured for horizontal and vertical movements.
2. Basal function and Irwin neurological assessment (day 2): Mice are assessed for weight, hindlimb clasping, righting reflex, vocalizations, fur, gait.
3. Balance Beam (day 3): Mice are exposed to two different beams of different diameters and timed for latency to cross.
4. Rotarod (RR)(day 4-7): Mice are trained for the RR with 4 exposures fixed at 5 rpm. Mice are tested for 4 trials/day for 3 consecutive days at 5 to 40 accelerating rpm for 300 seconds.
   These assays generated the Alfy loss-of-function data presented in FIG. 6. Breeding is staggered to permit testing to begin with N171, followed by zQ175 then BACHD. In light of the successful recapitulation of the cell based work thus far with Alfy loss-of-function, and the data represented in FIG. 2 cellular models may be similar predictive regarding Alfy over-expression.

Decreased aggregation likely correlates with better neuropathological and behavioral outcomes. Increased Alfy levels increase aggregate clearance and delay age of onset. To ensure that a positive outcome is due to increased protein turnover, transcript levels of mHtt are determined, e.g., to not have changed due to increased Alfy levels, thereby leading to fewer aggregates due to less total protein being formed. Alfy might also shuttle other cargoes such as mitochondria for degradation. The proteomic analyses of autophagosomes purified from these mice, and of the immunoprecipitation of hAlfy (via the 3xFLAG tag) may determine if the turnover of cargoes other than aggregated proteins underlies this beneficial effect.

Stereological analyses are performed using parameters that ensure a Gundersen coefficient of error of less than 0.05. Power analyses for all studies were performed using G*Power3. Minimum effect sizes were established based on variability characterized by published material on the mice. To ensure blinding to genotype, mice are only identified by their 8-digit AVID chip number. Genotypes are revealed once studies are completed. Age, genotype, strain and sex are considered as relevant biological variables. HD mice already show well known sex-dependent differences in open field and rotarod. To avoid issues with strain, controlled breeding strategies and littermate controls are routinely used. The CAG repeat numbers for the HD models except BACHD are confirmed at the time when experimental cohorts are created, and confirmed upon unblinding.

### Augmenting Alfy Levels in Symptomatic Mice and Disease Progression

To determine mechanistically how aggregation impacts pathogenesis, this must be examined in a temporal context. For example, protein aggregation may initiate a disease process and be unimportant for maintaining pathology, or aggregation may be a chronic stress and continuously contribute to a pathogenic process. Alfy overexpression is initiated after symptomatic onset, to test the hypothesis that Alfy promotes the clearance of pre-existing aggregates in the brain and slow or diminish symptoms in symptomatic mice.

Create Mouse Models of HD with Adult Inducible Alfy.

N171 and zQ1751^{KI/KI} mice are used, but not the BACHD mice due to the intervening loxP sites. A tam-inducible Cre line, Actin^{CreERTM/+} is used (FIG. 3).

An Alfy inducible KI (iKI) model is created by crossing ROSA26^{flx-stophAfy/flox-stophAfy} with Actin^{CreERTM/+} Efficient excision is achieved after intraperitoneal (i.p.) administration of tam at 2 mg/26 g body weight (or 200 μl solution/26 g body weight) for five consecutive days. Brains (n=3) are harvested for western blot analyses daily for one week to establish how long it is required to reach maximum levels of Alfy. Efficient tam-mediated excision occurs as late as 18 months of age (10 and 7mo. shown, FIGS. 3-6) for the Alfy inducible KO. The Alfy iKi mice are bred onto an HD background.

### Alfy-Overexpression and Disease-Course in the Symptomatic Mice.

Given the number of animals for this overall study, a single timepoint at which Alfy overexpression is induced is used. Induction is initiated when N171 and zQ175KI/KI demonstrate clear aggregation and symptomatic onset. Based on the literature, N171 is induced at 3.5 m/o and zQ175 at 8 m/o. The requirement is mice show deficits across two different behaviors and documented accumulation of insoluble protein by western blotting, and aggregation by IHC. The injection protocol begins at least 2 weeks prior to their next behavioral assessment. Two cohorts of animals are used, cohort1 is longitudinal, and cohort2 is for correlative purposes.

The behavior battery, neuropathology and aggregate staining are identical to those described above. For the behavioral battery, cohort1 mice are monitored for at least two rounds prior to the injection of tam, then at least 3 rounds of behavior afterwards, whereas cohort2 mice are represented by one timepoint prior to tam injection, and two afterwards. To limit the number of mice, the genotypes are AlfyiKi positive mice in the presence or absence of HD. Half of the animals are injected with tam, the other with vehicle control. Neuropathology and assessment of aggregation are represented by pre-onset, onset pre-injection, post-injection, late post-injection time points. Cumulative survival is also monitored. In the biochemistry cohort, excision efficiency of Alfy is monitored.

The benefit that might be gained from Alfy may be an age-dependent phenomenon, and once the brain becomes older, the benefits might lessen or be lost. The age at which overexpression is induced is still within breeding age, which should minimize this event. A small cohort of mice in which the Alfy iKI:HD mice are ‘activated’ at different ages, regardless of symptoms, is pursued. These mice are used to measure aggregation and Alfy levels. If an age-dependent effect is observed, an earlier tam-injection time is employed for complete behavioral and neuropathological analysis.

### The Mechanism of Action of the Disease Variant Alfy(13032V).

Although an inverse correlation between CAG repeat length and age of onset (AO) is well-appreciated (Andrew et al., 1993), analysis of only the most common repeat lengths of comparable size revealed that this relationship is significantly weakened, and both environmental and genetic modifiers can exert significant influence on AO (Wexler et al., 2004). A GWAS study identified several potential genetic modifiers within the Venezuelan cohort (Gayan et al., 2008) including a peak at 4q21, the genetic location of WDFY3, the gene that encodes for Alfy. Further analyses by Wexler, Housman and colleagues recently revealed that a variant in Alfy encoded by an isoleucine to valine change at amino acid 3032 (13032V) significantly correlated with an average of 10-year delay in AO (FIG. 9A). As shown in FIG. 10, brains collected from several carriers of the Alfy(13032V) variant were obtained. Immunoblotting revealed a significant increase in the levels of Alfy (FIG. 10A-C). This observation suggested that this variant increases Alfy function, leading to the delayed AO. To test this hypothesis directly, the Alfy(13032V) KI model is crossed to a mouse model of HD (FIG. 101).

### Characterization of Alfy(13032V) Mice

The conservation between human and mouse Alfy is greater than 90% identity, and the exon in which the nucleic acid having the Alfy(13032V) point mutation is observed is conserved. Alfy(13032V) mice are charactered as well as the impact on outcomes in the BACHD mouse model where the complete human HD gene is maintained.

Alfy(13032V) mice are crossed into the BACHD mouse background, such that the resulting littermate offspring (BACHD, BACHD::Alfy^{I3032V/+}, BACHD::Alfy^{I3032V/I3032V}) are analyzed at 3, 6 and 12 m/o. Like cohort 2 in Aim 1, N=12 mice/genotype/age are assessed behaviorally prior to being sacrificed by IHC (n=8) and immunoblotting (n=4). In addition to probing for detergent-soluble and -insoluble Htt, Alfy levels are probed. The BACHD negative mice are characterized in all assays to determine the impact of the 13032V mutation.

The 13032V mutation is present within the PH-BEACH domain of Alfy (FIG. 1), which is important for the interaction of p62/SQSTM1. Although this is not a dramatic amino acid change, changes between these two amino acids can alter protein-protein interactions (Dukare & Klempnauer, 2016; Yuan et al., 2010) or protein stability (Svane et al., 2016) which might explain why there is higher Alfy levels.

### Alfy(13032V) and the Cellular Phenotype in Human Neuron Model of HD.

Across several publications, the Yoo lab has established a model of HD, which is created from the microRNA-dependent directed conversion of patient dermal fibroblasts into medium spiny neurons (MSNs) or cortical neurons (Abernathy et al., 2017; Victor et al., 2014; Victor et al., 2014). Unlike iPSC-derived neurons (Christian et al., 2012), one of the most unique aspects of this MSN model is that it demonstrates aggregation of endogenous mHtt (Victor et al., 2018). What is most exciting is that this aggregation is present in neurons derived from patient fibroblasts collected from patients within the most common disease range (For example, FIG. 7 is from a patient with 46Q). In addition, they demonstrate a cell autonomous phenotype consisting of mutant htt-dependent DNA damage, mitochondrial dysfunction, and spontaneous degeneration over time. Together with the Yoo lab, we find that the depletion of Alfy in this model accelerates aggregation (FIG. 7).

To determine the impact of the Alfy(13032V) variant, CRISPR-Cas9 gene editing is used to introduce the Alfy(13032V) mutation into the rigorously characterized fibroblast lines from the Yoo lab. Homozygous mutations are the most likely outcome. Alfy KO is created to serve as a methodological control. To ensure the ability to screen for valid clones, a sequence for the restriction enzyme site Xbal has been included, so that a diagnostic digest can be used after PCR amplification. A minimum of three clones is selected for each successful line to submit for Next Generation Sequencing (NGS) at the Genome Center Core Facility at CUMC. The CRISPR-Cas9 D10A nickase, which induces CAG-repeat contractions to create isogenic controls (Cinesi et al., 2016) may be used. PCR of the CAG mutation, commonly used to monitor the CAG expansion status in mice, are used to confirm contraction. A subset of clones is confirmed by NGS.

Validated cell lines are differentiated into MSNs as described (Victor et al., 2018) (FIG. 7). Briefly, lentiviral preparation of a dox-responsive synthetic cluster of miR-9/9* and miR-124 (Yoo et al., 2011) as well as transcription factors cloned downstream of the EF1a promoter were used to transduce these cells as previously reported to generate cells analogous to human medium spiny neurons (Victor et al., 2014; Richner et al., 2015). Infected human fibroblasts are maintained in 15% FBS DMEM media for 5 days with doxycycline before re-plating onto coated coverslips. Cells are then selected with appropriate antibiotics in neuronal media (ScienCell) supplemented with valproic acid (1 mM), dibutyryl cAMP (200 μM), BDNF (10 ng/mL), NT-3 (10 ng/mL), RA (1 μM), and RevitaCell (100×). Dox is replenished every two days and media changed every 4 days. At day 19 of reprogramming, cells are analyzed. MSNs are stained for EM48, MW8 and 1C2 to quantify aggregation, and mitochondrial dysfunction, cell death and DNA damage are characterized as described (Victor et al., 2018). Total Alfy levels is examined both in the fibroblasts and in the neurons, to determine if a detectable change is present.

Little is known about basal autophagic function or how autophagy might be impacted in these fibroblast lines (for a review of autophagy and HD please see (Croce & Yamamoto, 2018)). For example, recent studies have implicated Htt to function in autophagy (Steffan, 2010; Zheng 2010), and it is unclear if a repeat number of 46 is sufficient to impede Htt function. The molecular events underlying autophagy may be investigated to determine if a loss of function of Htt might play a role. Experiments deleting Alfy levels suggests that the function of Alfy readily translates across the two models.

For over-expression based approaches the mutation in Alfy and AlfyC (C-terminal Alfy, see (Eenjes et al., 2016)) constructs has been introduced, and the edited fibroblasts from the control patients can be examined to determine if this variant specifically effects the turnover of mHtt but other aggregation prone proteins that require Alfy for clearance, such as alpha-synuclein (Filimonenko et al., 2010), SOD1(G93A) (Han et al., 2015), and ALIS (Clausen et al., 2010) may be investigated.

### Establish the Molecular Mechanism Underlying Alfy-Mediated Selective MA.

Alfy might drive aggregate-clearance, however many molecular questions still remain about selective MA of aggregates. For example, central to selective MA degradation is the capture of discrete cargo, and this cargo-selectivity is achieved by adaptor proteins. The current model suggests that adaptor proteins recognize, sequester and package substrates into the AV by scaffolding between an AV-associated protein, typically LC3, and the ubiquitinated substrates (FIG. 7) (Yamamoto & Yue, 2014; Mijaljica et al., 2012). Additionally, although several adaptor proteins have been identified, they share cargoes in common: the adaptor proteins p62, Nbr1 and Optn scaffold protein aggregates, mitochondria, peroxisomes and pathogens. Finally, all of the different adaptor proteins are proposed to function simply through its scaffolding activity. One hypothesis is that there is an alternative model of selective MA, in which these common adaptor proteins act as the core machinery that works in concert to promote cargo sequestration and AV membrane-building, whereas unique adaptor proteins, such as Alfy achieve cargo-specificity. Alfy is essential for aggregate clearance. Nonetheless, the role of other adaptor proteins, including p62, NBR1 and optineurin (Optn) which have also been implicated in aggregate-turnover, is not clear.

### Determine Adaptor Proteins for Aggregate Clearance.

Stable cell lines that have aided the understanding of Alfy function, are used to determine if these well-studied adaptor proteins are involved in aggregate clearance. WA combination of a loss-of-function (si or shRNA-mediated KD), and gain-of-function approaches (cDNA overexpression) are employed to determine if Optn (48), p62(49), NBR1(50-52), NDP-52(53) and WDR81(54) works with Alfy in aggregate clearance.

The two assays employed are a tet-regulated assay that expresses a mCFP-tagged fragment of Htt (Yamamota et al., 2006), and the newly created HaloTag(HT) stable cell line that expresses an exon1Htt fragment (FIG. 2) (Eenjes et al., 2016). Gene is monitored both by immunofluorescence and filter trap as described (Eenjes et al., 2016; Filimonenko et al., 2010). In addition to this mini-screen based approach, the HT-cell line is used to determine the relative localization of these adaptor proteins to the different classes of aggregates that are present at any given time (FIG. 2) by immunofluorescence. When possible, antibodies against endogenous protein are used, but for WDR81, where over-expression is used. All experiments ware performed independently at least 4 times, and a minimum of 100 cells will be counted using macros designed on ImageJ. Finally using the tissue generated above, reciprocal immunoprecipitation experiments are performed (one of Htt either against the C-term or N-term) and against 3xFLAG-Alfy to determine if Alfy interacts with mHTT as well as with other adaptor proteins, and whether mHtt can be found with Alfy as well as other adaptor proteins.

Although two different proteins might impact total aggregate-load, and appear to be involved in aggregate turnover (Filimonenko et al., 2010; Vos et al., 2010), using a series of assays that examines both formation and clearance separately, proteins are identified that can impact aggregate load in different ways (Eenjes et al., 2016). The assays and approach described herein can determine whether the tested adaptor proteins are involved in aggregate-clearance, and whether they work with Alfy or not. Phospho-mimetic and phospho-dead mutants may be used to determine if function can be potentiated by over-expression or can rescue knockdown. The reciprocal co-IP experiments allow for determining the components of the greater Alfy-mHtt complex, but also determine if mHtt can interact with other adaptor proteins in the absence of Alfy, which may suggest either different temporal interactions, or that discrete complexes are being formed. For an unbiased approach, these immunoprecipitates, especially for 3xFLAGhAlfy (which can be readily controlled with IPs from 3xFLAGhALfy negative mice) can analyzed using LC-MS/MS.

### Determine the Importance of the Atg8 Orthologs for Aggregate Clearance.

One of the defining features of adaptor proteins is the presence of an interacting domain (LIR domains) with the Atg8 orthologs, such as LC3 and Gabarap (GR). Different LIRs have differential specificities for the different orthologs. For example, Alfy interacts specifically with GR (Lystad et al., 2014), whereas Optn and WDR81 might be specific for LC3C (Liu et al., 2017; Wild et al., 2011). It is hypothesized that the different Atg8 homologs might be indicative of different membrane sources contributing to AP formation around a relatively large cargo, such as aggregates. Therefore, tracking Atg8 orthologs might point toward how the different adaptor proteins might differentially contribute to selective autophagy.

It is determined how the different Atg8 homologs distribute using immunofluorescence against endogenous Atg8 orthologs. Cells are stained for the different Atg8 orthologs to determine how the distribution of these proteins might change in an adaptor protein-dependent manner.

Alfy can localize to LC3-positive structures so both LC3 and GR may co-localize to aggregated proteins. How that distribution might change (LC3 only, GR only, or LC3 and GR) and the presence and absence of different adaptor proteins may suggest that different membrane sources might be involved in membrane building. This would indicate why multiple adaptor proteins might be necessary, and the role of the FYVE domain of Alfy which has not yet been elucidated. In contrast however, we might see aggregates co-localize to different Atg8 homologs, and that in the absence of Alfy, GR+ co-localization is lost, suggesting that Alfy sorts aggregates into discrete vesicle structures.

Determine the Relationship of the Adaptor Proteins with Autophagy and Alfy.

A standard practice is to fractionate tissue and cells to enrich for autophagosomes (AP, FIG. 8) as a way to establish directly if cargoes are targeted for autophagy. Mice generated in the crosses are used to isolate APs to determine there is an increased amount of mHtt present upon increasing Alfy levels. Immunoblotting of these fractions together with a quantitative proteomic approach tests the prediction of the alternative model that increased Alfy levels can also increase other cargo adaptors to be captured in the AP.

Approach: Purified Avs are isolated (FIG. 8). Following tissue fractionation to enrich (Stromhaug et al., 1998), they are immuno-purified using a known marker of mature AVs, LC3. This can be accomplished using GFP-LC3 or in a two-step approach using antibodies against LC3B. Using quantitative mass spectrometry (MS), immunoblotting and cryo-electron microscopy (CryoEM), it was found that lysosomal enzymes and ER proteins deplete, and autophagy receptors such as p62 further enrich. Moreover, multilamellar vesicles, which is a structural hallmark of Avs, are enriched. For every prep, n=5 brains, which gives enough material for quality control, several runs by MS, and follow-up analyses by western blotting. Fractions of purified AVs are analyzed by detergent fractionation by differential centrifugation, IB and probed for N=3 replicates are performed at 5 m/o for all genotypes and 12 m/o for BACHD and zQ175. To assess quantitatively AV content, quantitative MS is performed at the CUMC Proteomic core, which has the capability to use isobaric tags for quantitative proteomic analysis. Briefly, proteins from each sample are digested and chemically labeled via 10-plex isobaric Tandem Mass Tags (TMT) and analyzed using nano-flow LC-MS/MS on a ThermoFisher Orbitrap Fusion Mass spectrometer. To achieve near comprehensive protein coverage, mixed AV prep samples are pre-fractionated by high pH reverse-phase HPLC to reduce sample complexity. Multiple fractions are analyzed for each 10plex sample mix. Peptide identification, filtering, and quantification are performed using a combination of commercially and publicly available software such as SEQUEST, Maxquant, and custom analysis tools for MS3 read-out. There is a focus on MA associated proteins, with a particular focus on proteins implicated in selective autophagy, as well as mHtt.

Immunoblotting is used to examine specifically the proteins of interest, and how their levels might change in the presence of Alfy, as well as LC-MS/MS. The former provides the basic information regarding the adaptor proteins, and Atg8 orthologs, as well as levels of detergent soluble and insoluble mHtt. The latter allows for a better unbiased perspective in regards to Alfy-dependent cargoes. Mitochondrial proteins are by far the most prevalent cargo from brain and interestingly, ALFY interacts with NIPSNAPs (Abudu, 2019), and thus if increased Alfy-levels change, it may be elucidated how two different cargoes (aggregates and mitochondria) might be sorted, as well as other Alfy-cargoes. In addition to determining how the adaptor proteins might react to increased Alfy, the presence of mHtt fragments and full-length proteins might impact the AV proteome.

### Example 2

### A SNP in WDFY3 Protects Against Neurodegeneration by Augmenting Selective Autophagy

A genetic linkage study in the Venezuelan HD Kindreds in which the HD gene was identified {Gusella, 1983 #235} previously reported several loci modifying the age of onset of Huntington's disease, including one at chr4q21{Gayan, 2008 #5356}. However, the mapping of this study to a yet small panel of single nucleotides polymorphisms (SNPs) across the genome was not able to resolve potential modifier genes of interest within that locus. Members of the kindred were genotyped with a fine-mapping approach that combined whole genome sequencing and an Illumina Core Exome SNP array across 440 HD patients with corresponding clinical data. A genome-wide association analysis was performed that signals for association with residual age of onset using a linear mixed model with covariates including ancestry characteristics and gender. This approach recapitulated a significant signal within chr4q21 specifically mapping to variants proximal and within the gene WDFY3 (FIGS. 9-10). The most significantly associated SNP, rs17368018 encoded a missense mutation (A>G) within the coding region of the gene (WDFY^{3rs17368018} (WDFY3^{rs})). The Venezuelan pedigree was examined for the patients who carry this signal and identified an extended family with primarily delayed ages of onset than expected by repeat size by 6-23 years (FIG. 9A).

### Recreation of SNP Rs17368018 in Mice Wdfy3 Recapitulates Disease Protection

Given the rarity of the variants, ˜0.9% in a Venezuelan population and ˜0.7% in the broader population, coupled with the rarity of HD, the likelihood of obtaining sufficient patient numbers presented substantial limitations. Thus to continue, an orthogonal approach using mice was employed. WDFY3 shares significant sequence identity with murine Wdfy3 with over 96% conservation (NCBI). The rsl7368018 variant was recreated in mice by creating the corresponding T>C change in the mouse genome (Alfy^{Var/+}) (FIG. 9B). Mice carrying this variant were indistinguishable from littermate wildtype mice in breeding and home cage behavior (data not shown), as well as in spontaneous locomotor activity in the open field maze (FIG. 9J).

To directly test the impact of introducing the SNP on disease onset, we crossed our variant (Alfy^{Var}) model into an HD background (FIG. 91). The heterozygous CAG140 knock-in mouse model (Menalled et al., 2002) was used because it is a widely used model of HD, and in contrast to transgenic models, maintains gene dosage. These mice demonstrate locomotor deficits beginning 6 months old (m/o), and demonstrate pathological changes in medium spiny neurons of the striatum, which are vulnerable in this disease.

Consistent with previous reports, both 6 m/o female and male CAG140 mice demonstrated a significant hypolocomotor phenotype when compared to littermate controls (FIG. 9C). Notably, introduction of the Alfy^{Var} into CAG140 mice was sufficient to delay the onset of this phenotype, making CAG140:AlfyVar mice of both sexes indistinguishable from littermate controls (FIG. 9C). In light of these findings, the mice were examined for neuropathological indications of neuroinflammation such as reactive astrocytosis and microgliosis, using GFAP and Iba1 staining, respectively (FIG. 9D,E). CAG140 striata demonstrate a profound astrocytosis and clear microgliosis at 6 m/o, but co-expression of Alfy^{Var} significantly blunted these indications of neuroinflammation. Moreover, we found that the medium spiny neuron marker FoxP1, which is significantly diminished in CAG140 mice, is also maintained in the presence of Alfy^{Var} (FIG. 9F). Taken together, these data indicate that recreation of the rs17368018 variant in mice recapitulated observations in patients, suggesting that this SNP confers protection and delays the onset of HD.

rs17368018 is found in a coding exon of WDFY3, which encodes for the protein ALFY, an adaptor protein that traffics aggregates for degradation by selective autophagy (Filimonenko), including in cells of the adult brain (Fox et al.). Next it was determined if Alfy^{Var} affected the aggregation load in CAG140 mice (FIG. 33G). Immunohistochemistry followed by stereological quantification revealed that in addition to protection against the HD phenotype, there was a significant reduction of mHtt inclusions in CAG140::Alfy^{Var} mice. These data suggest that this variant might augment Alfy function and confer protection by augmenting aggregate-turnover by autophagy.

### Rs17368018 Increases Expression of the Selectivity Autophagy Adaptor Protein Alfy

In cell-based systems, it was reported that increasing Alfy levels can augment the turnover of preformed aggregates. The set of significant variants in linkage disequilibrium in the WDFY3 locus was examined to determine whether any of the variants affected gene expression of WDFY3 or any neighboring gene in the locus. Since the rarity of the variants precluded inclusion in any eQTL database, publicly available epigenetic data in the WDFY3 locus around the significant modifier variants was examined. A brain-specific enhancer was identified around rs17368018 as profiled by H3K27ac data (FIG. 10O). In addition, single cell ATAC-seq data in human cortex revealed an excitatory neuron-specific peak of chromatin accessibility around rs17368018 (FIG. 10O). To determine if these observations were associated with a change in WDFY3 expression, 200 randomly obtained cortices from the New York Brain Bank were genotyped to identify carriers of rs17368018.6 independent samples of varied patient histories were identified. Next, quantitative RT-PCR was performed on both WDFY3^{WT} and WDFY3^{S} carriers (FIG. 10A). RT-PCR revealed increased levels of WDFY3 expression in heterozygous individuals genotyped with the rs17368018 variant compared to controls (FIG. 10A). To determine if these observations translated to protein, western blot analyses were performed for Alfy on cerebellar samples obtained from these patients (FIG. 10B,C). It was found that similarly to the qRT-PCR results, rs17368018 carriers expressed higher levels of Alfy.

To confirm if this SNP was sufficient to increase Alfy expression, the Alfy^{Var} mice were examined. Strikingly, knockin of the SNP mice recapitulated the human data (FIG. 10D-F), leading to the increase of both the transcript and the protein levels of the gene product of Wdfy3, Alfy. Based on these findings, it was hypothesized that the protective mechanism of the SNP is through increasing Alfy levels of expression.

### Ectopic Upregulation of Alfy is Sufficient to Recap

A second model was created that ectopically overexpresses Alfy by introducing into the Rosa26 locus a sequence consisting of the full-length human ALFY (hALFY) cDNA preceded by a 3xFLAG-tag (Rosah^{Alfy}) (FIG. 10P). We used the endogenous Rosa26 promotor to drive expression. Rosa^{hAlfy/+} broadly expresses Alfy including in the brain (FIG. 10G, FIG. 10R, and data not shown). Although heavily conserved, it was sought to confirm that hALFY can complement murine Alfy. To do so, it was determined if Rosa^{hAlfy/+} could rescue the perinatal lethality and severe midline crossing deficits we previously observed in Alfy KO mice (dragich 2016) (FIG. 10R, S). The data revealed the ectopic overexpression of human Alfy can complement mouse Alfy by rescuing both the perinatal lethality and midline crossing defects. Moreover, we find that the NH3-terminal 3xFLAG tag does not interfere with Alfy function.

To test if ectopic overexpression of Alfy is sufficient to capture the effects of the Alfy variant, the mice were crossed to the CAG140 model (FIG. 101). Consistent with the findings in Alfy^{Var}, Alfy overexpression was protective across all measures, including the behavioral (FIG. 10J) and neuropathological (FIG. 10K-N) changes observed. Consistent with observations in cells, ectopic overexpression of Alfy also led to a significant decrease in mHtt accumulation in the CAG140 brain (FIG. 10K). Taken together, these data indicate that overexpression of Alfy is sufficient to recapitulate our observations in the Alfy Var mice.

Alfy Upregulation is Protective by Increasing the Turnover of Aggregated Proteins by autophagy

In addition to the aggregation of mHtt, the CAG expansion mutation has been implicated to evoke other toxic events that may lead to HD, such as cause the loss of function of the endogenous 350 kDa protein, enhance protein-protein interactions, or change transcription. To examine further if the protection due to increased Alfy levels is due to the clearance of mHtt aggregates, the N171-82Q model, a model of HD that expresses a short fragment of Htt with 82 glutamines driven by the prion promotor (Schilling, 1999), was used. Due to the limited length of the Htt protein expressed, it has been postulated that the aggressive phenotype is primarily driven by the aggregation of the polyglutamine (polyQ) stretch itself. Crossing N171-82Q mice with either Alfy^{Var} or Rosah^{Alfy} mice created the experimental groups that were processed for the same behavioral and neuropathological outcomes for CAG140 (FIG. 11G). N171-82Q mice present with cortical and striatal intranuclear mHtt inclusions beginning at 4 m/o. Crossing the N171-82Q mice to either AlfyVar or Rosa26 Alfy^{Var} or Rosah^{Alfy} hAlfy mice appeared to diminish the presence of aggregates in both brain regions (FIG. 11A,H). Stereological quantification for aggregate load in the striatum confirmed the significant reduction (FIG. 11A).

Subsequent neuropathological analyses also revealed that the loss of aggregation was again accompanied by the loss of signs of neuroinflammation (FIG. 11B,C), and a rescue of FoxP1 expression (FIG. 11D). Behavioral analyses revealed that increased Alfy expression also delayed the onset of the hypolocomotor phenotype (FIG. 11E), as well as delay the latency to fall on the accelerated rotarod (FIG. 11J). Thus, similarly to the CAG140 model, increasing levels of Alfy augments aggregate turnover, and protects against the neuropathological outcomes of the expanded polyQ.

A unique feature of the N171-82Q model is that they suffer from premature lethality. It is uncertain what causes the early demise of these mice, but a similar design that models aggregation of TDP-43 has a severe gut motility defects that leads to death. Monitoring fecal deposits and pathologic examination suggests that gut motility deficits might also be playing a role in the N171-82Q model as well. Interestingly, overexpression of Alfy leads to a significant lifespan expansion of the N171-82Q model (FIG. 11F). Notably in this assay, the presence of the Alfy variant was more effective than ectopic Alfy overexpression alone. Differences in the activity of the Wdfy3 and Rosa26 promoters might explain this difference.

### Upregulation of Alfy is Protective in a Mouse Model of Synucleinopathy

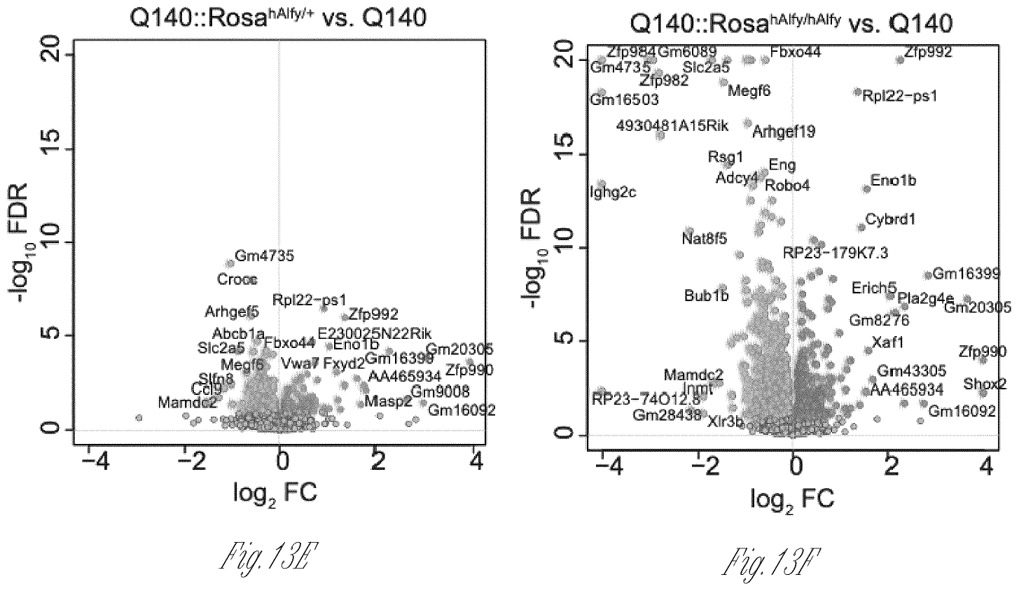

Although the findings in the N171 model suggest that Alfy overexpression is protective by augmenting aggregate-clearance, it was speculated that the protective effects should extend to different types of inclusions. One oligomer that is often considered toxic is aggregated a-Synuclein (aSyn). Next it was tested if Alfy overexpression can protect against the toxicity evoked by intrastriatal injections of preformed fibrils (PFF) of α-synuclein (Luk 2012, Paumier 2015, Peelaerts 2015). 4 m/o wild-type and Rosa^{hAlfy/hAlfy} mice received a single, unilateral injection of PFFs of mouse aSyn and were euthanized 120 days post-injection (FIG. 12A). As it has been shown previously, PFF-injection in wild-type mice leads to the accumulation of phosphorylated aSyn (pS129-aSyn) in the injection area in the striatum, as well as several brain regions that innervate the striatum including the cortex, amygdala, and substantia nigra pars compacta (SNpc) (FIG. 12C, J, K, L). Although the aggregation load in the striata of PFF-injected Rosa^{hAlfy/hAlfy} mice was similar to the load in wildtype mice (FIG. 12J), the level of aSyn accumulation appeared attenuated in the projection areas. Subsequent quantification in SNpc revealed that indeed there was significantly less aggregation in the RosahAlfy mice (FIG. 12C-D). These data demonstrate that Alfy overexpression can also augment the clearance of aSyn aggregates.

It was next asked if attenuating aggregate accumulation was neuroprotective and preserved dopaminergic neurons in the SNpc. Quantification of tyrosine hydroxylase (TH)-positive or Nissl positive neurons in the SNpc showed degeneration of dopaminergic neurons in wild-type animals, consistent with previous findings (Luk, 2012; Paumier, 2015)(FIG. 12E-G). In Rosa^{hAlfy/hAlfy} mice, the DA neurons were significantly spared (FIG. 12G-I). In sum, these findings demonstrate that Alfy overexpression reduces aggregate burden and delays the neurodegenerative α-synuclein PFF model of PD. Importantly, our work suggests that the protective action of Alfy overexpression can be applied across different proteinopathies.

### Alfy Overexpression Exhibits a Dose-Dependent Effect on the Transcriptome in an HD Background

The abnormal accumulation of protein is a hallmark of the vast majority of neurodegenerative diseases, but their contribution to pathogenesis is unclear. The present findings suggest that protein accumulation contributes to disease pathogenesis, and slowing accumulation can slow neurotoxicity. To gain insight into how diminishing aggregate burden might be protective, the CAG140 mice were examined, which through a series of studies, have a well-established transcriptional signature via bulk RNA sequencing (Langfelder 2016, Lee 2018) that appears indicative. The same approach was used to determine if the pattern of changes evoked by Alfy overexpression can give us insight into how aggregate-clearance might be protective.

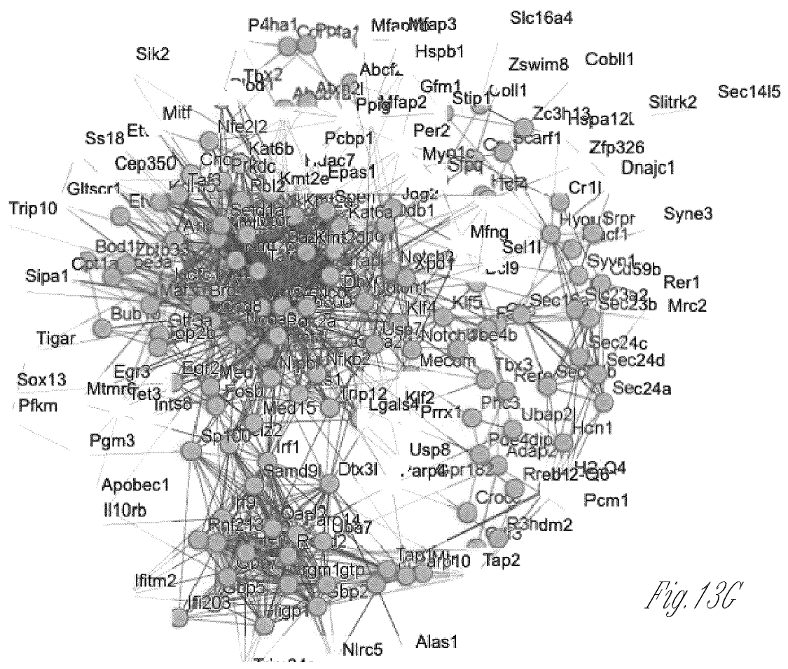

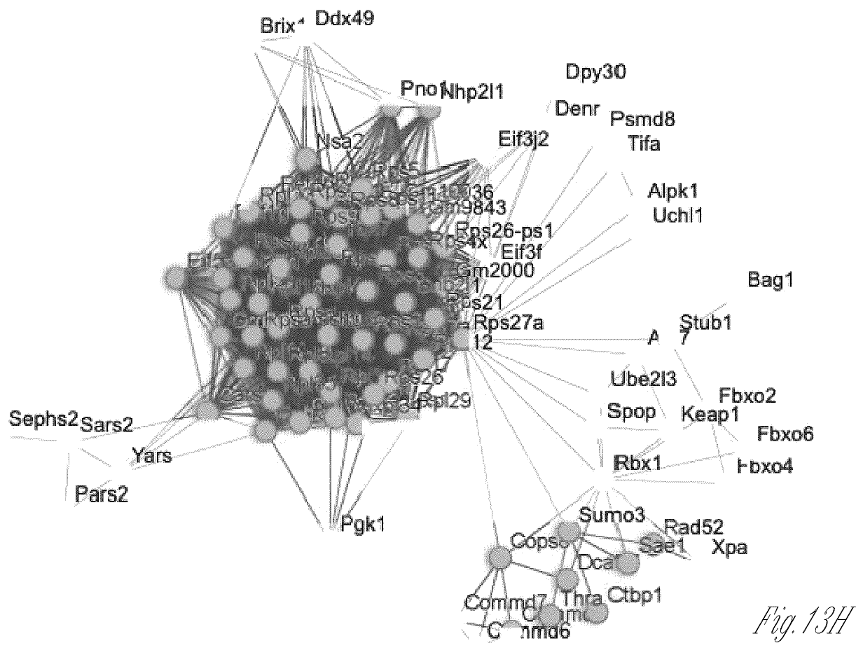

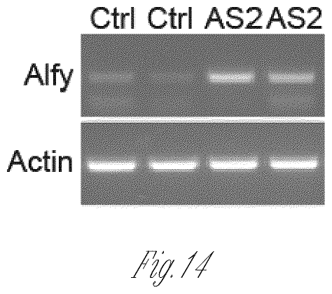

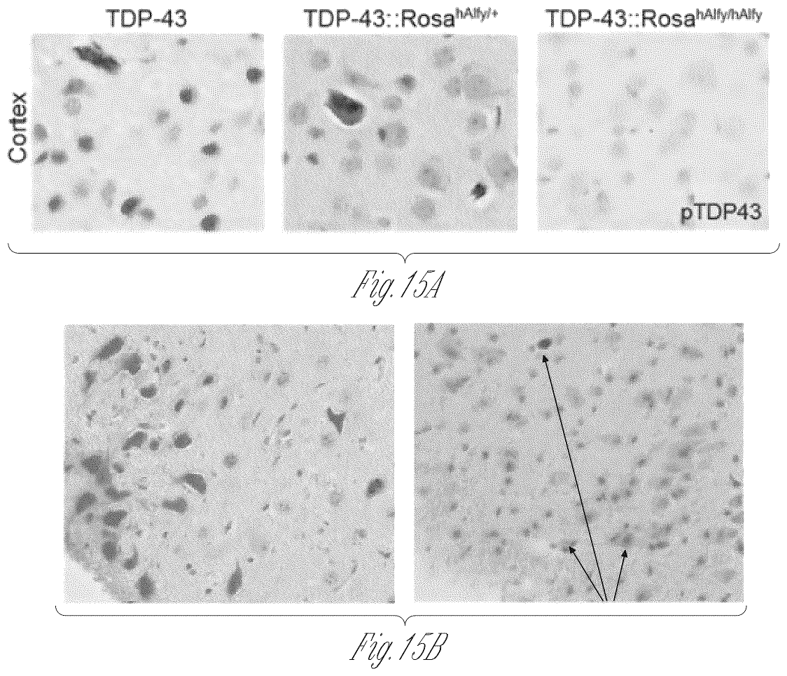

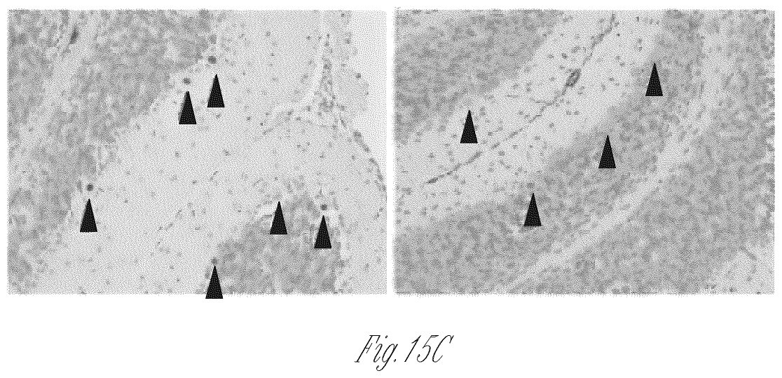

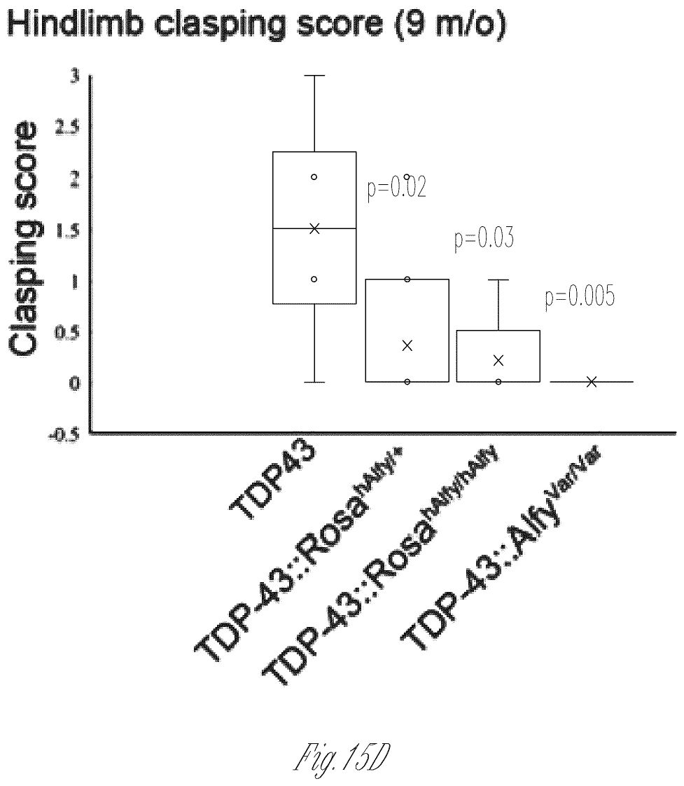

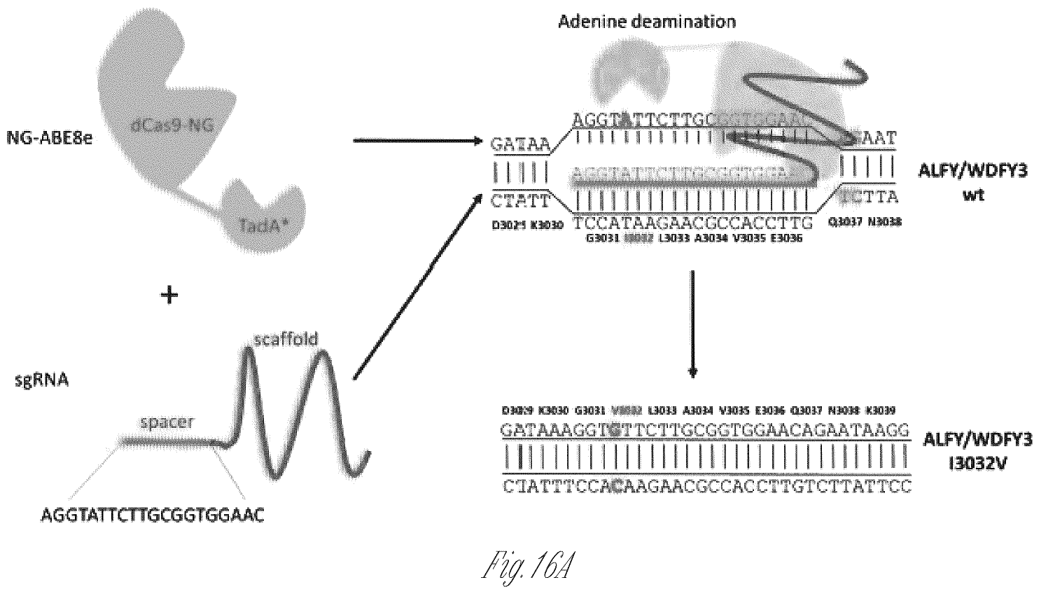

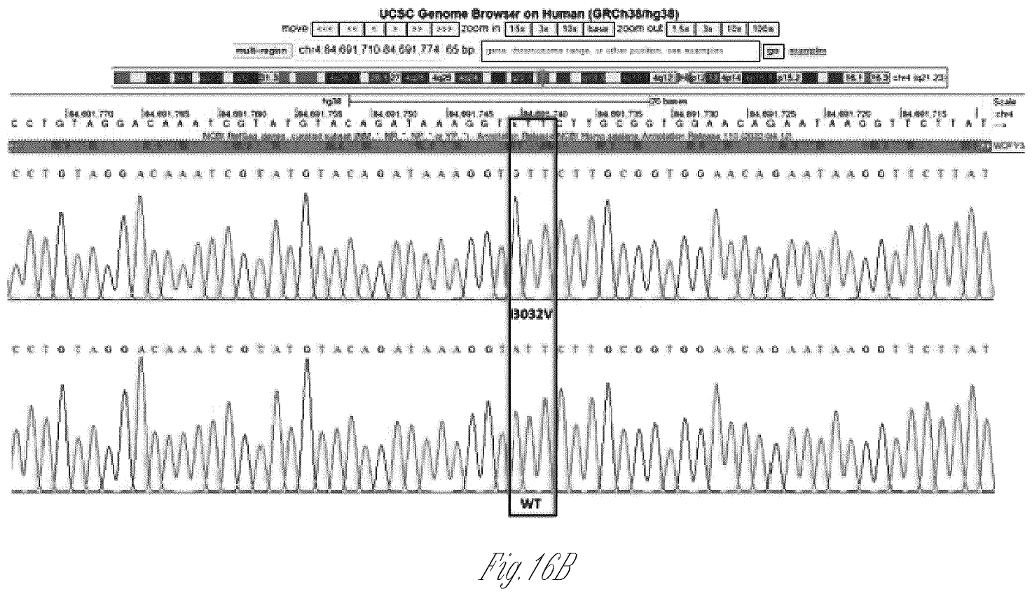

First it was examined the robustness of the transcriptional signature of the CAG140 striata, by determining to what extent it might differ from the CAG140 mice in our colony. Several features could influence the transcriptome: First, the present colony was independently started and established at Columbia University (denoted CAG140) rather than at UCLA (denoted Q140), the present colony was started several years later, and the mice were of mixed strain, since they were crossed with the RosahAlfy mice. Correlation analysis between the differential expression statistics of the CAG140 striata vs. littermate WT striata from Columbia comparison and the previously published comparison (Q140 vs Q20) (Lee, 2018) revealed that the Q140 maintained the transcriptional signature as previously reported, with a correlation coefficient of 0.78. (FIG. 13A). These data indicate that despite the many differences between the two colonies, the differential gene expression evoked by the CAG expansion mutation is very robust.

It was next determined if Alfy overexpression affected the transcriptional signature (FIG. 13B,C). Although the behavioral and neuropathological phenotype of the CAG140 mice did not significantly differ from wildtype, the transcriptional signature was not moved by the presence of one or two copies of Alfy: The comparison of CAG140 vs CAG140::Rosa^{hAlfy/+} or CAG140::Rosa^{hAlfy/hAlfy} to Q140 vs Q20 led to correlation coefficients of 0.093 or 0.024, respectively. This was consistent with principle component analysis, which identified CAG140 to account for the largest possible variance. Although the transcriptional signature was largely unchanged, a number of transcripts whose differential expression was exacerbated or reversed were identified (Tables 1 and 2), the numbers of which increased with greater Alfy levels. Metascape analyses of the affected genes revealed. These data suggest that the vast majority of transcriptional changes observed in the CAG140 mice may reflect a vulnerability- or disease-state caused by the CAG expansion mutation, the vast majority of changes do not reflect the phenotypic state of the mouse.

Although the transcriptional signature of CAG140 was not significantly altered by the expression of Alfy, the presence of Alfy led to discrete changes in differentially expressed genes (FIG. 13d). Notably, there was only a minimal enrichment of differentially expresses genes between Rosa^{hA/+} and wild-type controls (FIG. 13D). In contrast, we found a dose-dependent enrichment of differentially expressed genes when Alfy was overexpressed on the CAG140+/− background (FIG. 13E,F). It was sought to further explore the pathways and processes most affected in our CAG140^{+/-}::Rosa^{hA/+} and CAG140^{+/-}::Rosa^{hA/hA} data. Using Metascape, we identified enrichment heatmaps based on fold change (FIG. 13G) and protein-protein interaction enrichment analysis (FIG. 13H). The processes identified may be different than the processes affected in the CAG140 transcriptome, suggesting these are changes that are specific to Alfy. Specifically, CAG140^{+/-}::Rosa^{hA/+} and CAG140^{+/-}::Rosa^{hA/hA} data overwhelmingly showed significant dose-dependent upregulation of three major cell processes: translation, ribosomal biogenesis, and mitochondrial proteins, all of which have been shown to be dysfunctional in HD pathogenesis (Culver, 2012; Yang, 2016; Tauber, 2011; Hosp, 2017; Moily, 2017; Yablonska, 2019; Joag, 2020). It was next sought to validate the findings. Given that transcript levels of genes expressed in translation and ribosomal biogenesis pose little significance in their respective processes, immunoblotting was used to monitor protein levels directly.

### DISCUSSION

Abnormal protein accumulation is a pathologic feature among many neurodegenerative diseases including HD, PD, ALS, Alzheimer's disease, and frontotemporal dementia. These disorders are usually classified by a mutant protein or gene and are categorized into groups like tauopathies, synucleinopathies, or polyQ-disorders. However, diseases rarely present in a pure form; in most cases, there is concurrent expression of different mutant proteins such as α-syn, tau, and TDP-43 (Mattila 1998, Irwin 2013, Teravskis 2018, Jo 2020). Therefore, reducing these diseases to a single proteinopathy-type may not be advantageous. Rather than focusing on the discrete protein or gene which might assign disease specificity, it is important for therapeutic strategies and treatments to target the mechanisms underlying protein accumulation and cell homeostasis.

By combining human genetics, mouse genetics, and biochemistry, we have established the therapeutic strategy of increasing Alfy in vivo to mitigate pathology across several proteinopathies. The present data provides insight into the relevance of diminishing protein-mediated toxicity and therapeutic outcome in the adult brain. The significant potential of augmenting the disease modifier, Alfy, to alleviate aggregate burden and be neuroprotective against multiple proteinopathy models is disclosed. This was directly tested using a genetic approach to study Alfy overexpression in vivo. Animals overexpressing Alfy in an HD background showed a rescued phenotype. Data showed diminished aggregation, increased motor function, reduced astrogliosis and microgliosis, and extended survival by several weeks. At the transcriptional level, Alfy overexpression in a disease context upregulated genes involved in ribosomal biogenesis and Wnt signaling, highlighting Alfy's impact of reducing aggregate burden to re-establish cellular homeostasis.

In parallel, a human variant, p.13032V Alfy, was identified that is strongly correlated with delayed onset within the HD population. By modeling this mutation, the present findings are consistent with human data that p.13032V Alfy has increased Alfy expression levels and can ultimately mitigate disease. Consistent with the hypothesis of higher Alfy levels being neuroprotective, p.13032V Alfy was found to improve motor function, reduce aggregate burden, and extend survival by several months in HD mice.

Moreover, not being limited to only HD, it was demonstrated through two independent proteinopathy models that Alfy overexpression is protective against disease pathogenesis. Taken together, these findings have shown Alfy's ability to reduce inclusions of mHtt, α-syn, and TDP-43 mutant protein, ultimately leading to protection against toxicity.

Upregulating Alfy expression has exciting therapeutic potential because it is not limited to a single proteinopathy; therefore, opportunities for implementation are extensive.

TABLE 1
Significant Rescue (FDR < 0.10)

| Q140 vs. Q140::RosahAlfy/+ | | Q140 vs. Q140::RosahAlfy/hAlfy | |
|---|---|---|---|
| Down for | Up for | Down for | Up for |
| Q140 vs. WT | Q140 vs. WT | Q140 vs. WT | Q140 vs. WT |
| Vwa7 | Fbxo44 | Smarcd3 | Fbxo44 |
| E230025N22Rik | Polr2a | Zfp706 | Cntn6 |
| Sertad1 | Prkdc | Zfp106 | Sec23b |
| Eno1b | Pcdhb2 | E230025N22Rik | Polr2a |
| Slc37a4 | Sec23b | Rnf207 | Eif4g3 |
| N4bp3 | Cdh2 | Cyp2a5 | Atad2b |
| Blvrb | Masp2 | Eno1b | Nap1l2 |
| Psd | Dock9 | Vwa7 | Dock9 |
| Zfp706 | Fyco1 | Ptpn7 | Synj1 |
| Rpl18a | Lrrc9 | Rpl18a | Dhx57 |
| Lrrc1 | Dscaml1 | Nod2 | Tbc1d22b |
| Rps6kl1 | Dlg1 | Slc37a4 | Pdk1 |
| Ddi2 | Ccar1 | Usp9x | Taf1 |
| Brk1 | Pla2g4a | Fbxo2 | Tpra1 |
| Mettl9 | Fry | Hrk | Ralgapa1 |
| Usp9x | Asap3 | 1700037H04Rik | Cdh2 |
| Aim1l | Trip11 | Blvrb | Itgav |
| 9330111N05Rik | Nemf | Arhgef4 | Pcdhb2 |
| Smim7 | Cdc23 | 9130401M01Rik | Cttn |
| Arhgef15 | Arhgap26 | Fam213b | Arfgef2 |
| Sgsm2 | Ube4a | St3gal5 | Slc38a6 |
| Gm15421 | Kank1 | Cdk4 | Zgrf1 |
| Pim3 | Rsl24d1 | Ddit3 | Srpk2 |
| Utp18 | Mtor | Rnd2 | Bpnt1 |
| Qdpr | Thsd1 | Gpr34 | Pcdh20 |
| Rps5 | Acaca | Pdzd4 | Baz2b |
| Gid4 | Hcn1 | Smyd5 | Dmxl2 |
| Fgfbp3 | B230209E15Rik | Mettl9 | Ccar1 |
| | | Brk1 | Pnpla6 |
| | | Dmxl1 | Nub1 |
| | | Fkbp8 | Pcdhga4 |
| | | Adra2c | Pla2g4a |
| | | Phactr3 | Fry |
| | | Triap1 | Adgrl2 |
| | | Aim1l | Kif21a |
| | | Tmem138 | Ltbp4 |
| | | Dhrs3 | Herc3 |
| | | Necab2 | Taf2 |
| | | Rgs10 | Nemf |
| | | Sh3bgrl3 | Cse1l |
| | | Chmp3 | Vps13a |
| | | Ptpmt1 | Xpo1 |
| | | Slmo1 | Pcdh10 |
| | | Eno2 | Cdc23 |
| | | Slc35b4 | Sh3bp1 |
| | | Rpl27a | Itfg2 |
| | | Ube2q2 | Nrip2 |
| | | Clec12a | Cr1l |
| | | Snn | Tbc1d8b |
| | | Anapc13 | Nap1l3 |
| | | Eif5a | Arhgap26 |
| | | Mgat4b | Mpdz |
| | | Pdrg1 | Zfc3h1 |
| | | Fam110b | Abcf2 |
| | | Ssbp4 | Hipk3 |
| | | Gsr | Wdfy3 |
| | | Gna13 | Atrx |
| | | Tacr1 | Faxc |
| | | Zcwpw1 | Stk3 |
| | | Txnl4b | Dync1i2 |
| | | Smim7 | Gabra1 |
| | | Arhgef15 | Lrrn1 |
| | | Mppe1 | Trpc4 |
| | | 4-Mar | Prkdc |
| | | Cgref1 | Plekhh2 |
| | | Rnf7 | Myo1c |
| | | Hmgn1 | Ndufa5 |
| | | Nit2 | Ddx46 |
| | | Sgsm2 | Arfgef1 |
| | | Cdh9 | Fat1 |
| | | Neurl1a | P2rx4 |
| | | Gnl1 | Exph5 |
| | | Anapc16 | Ppp5c |
| | | Nat14 | Top2b |
| | | Sh3bgrl2 | Ube4a |
| | | Rpl29 | Dlg1 |
| | | Inafm2 | Ipo5 |
| | | Ing5 | BC005561 |
| | | Lipo3 | Magee1 |
| | | Stard10 | Dlc1 |
| | | Smchd1 | Hdac7 |
| | | Parp4 | Xpo5 |
| | | D2Wsu81e | Slitrk4 |
| | | Ctxn1 | Tmem63b |
| | | Zfp882 | Arl5a |
| | | Lnpep | Soga3 |
| | | Svil | Map4k2 |
| | | Jph4 | Fam161a |
| | | Ccdc88a | Tpr |
| | | Camk1d | Kank1 |
| | | Psd | Phf2 |
| | | Parp6 | Abcd2 |
| | | Pcm1 | Arhgef11 |
| | | Cbr3 | A230046K03Rik |
| | | Gabrb2 | Asap3 |
| | | Atg12 | Fam114a2 |
| | | Phip | Fam126b |
| | | B3galnt2 | Pcsk1 |
| | | 1700048O20Rik | Rsl24d1 |
| | | Rps6kl1 | Cpsf6 |
| | | Chmp4b | Pcdha11 |
| | | Icmt | Hcfc1 |
| | | Tom1l1 | Mtor |
| | | Atp1b3 | Mrpl27 |
| | | Ppp1r1a | Trip12 |
| | | B3galt1 | Chd8 |
| | | 1190005I06Rik | Etv4 |
| | | Gxylt1 | Mllt4 |
| | | Thg1l | Nnt |
| | | Kcns2 | Cct8 |
| | | Mkrn2 | Stra6 |
| | | Wrb | 6-Mar |
| | | Utp18 | Thsd1 |
| | | Ube3a | Acaca |
| | | Sumo3 | Numa1 |
| | | Qdpr | Scaf4 |
| | | Tmem158 | Gfm1 |
| | | Zbtb48 | Hcn1 |
| | | Setd6 | Rusc2 |
| | | Bod1 | B230209E15Rik |
| | | Rps5 | Dcaf11 |
| | | Nupr1 | |
| | | Ugt8a | |
| | | Pars2 | |
| | | Wdr89 | |
| | | Tma7 | |
| | | Gmeb1 | |
| | | Gid4 | |
| | | Cspg4 | |
| | | Ncf1 | |
| | | Fgfbp3 | |
| | | Tmcc1 | |
| | | Ncam2 | |

TABLE 2
Significant Exacerbation (FDR < 0.10)

| Q140 vs. Q140::RosahAlfy/+ | | Q140 vs. Q140::RosahAlfy/hAlfy | |
|---|---|---|---|
| Down for | Up for | Down for | Up for |
| Q140 vs. WT | Q140 vs. WT | Q140 vs. WT | Q140 vs. WT |
| Crocc | Ptpa | Crocc | Nicn1 |
| Flt1 | 4930563E22Rik | Ryr1 | Rhov |
| Abcb1a | B4galnt4 | Flt1 | Ube2l3 |
| Cdh5 | Arhgdig | Ptprb | Vsig2 |
| Ptprb | 2210016L21Rik | Esam | 4930563E22Rik |
| Slc25a34 | Pop4 | Oscar | Timm22 |
| Itih5 | Cpt1a | Abcb1a | Ptpa |
| Cp | Kdr | Tmc4 | Pla2g4b |
| Adgrf5 | E2f2 | Adgra2 | Sord |
| Stab1 | Srd5a1 | Tns2 | Fahd2a |
| Dcn | Cecr5 | Lrrk1 | Degs2 |
| Serinc3 | Chrnb4 | Plxnd1 | Gcsh |
| Abca9 | Csf3r | Ahnak | 2210016L21Rik |
| Dock4 | | Dock6 | Mea1 |
| Cfh | | Cdh5 | Smim10l2a |
| Zmynd8 | | Flt4 | Gm5113 |
| Hmha1 | | Ccdc88c | Mdp1 |
| C1qb | | Stab51 | Eef1a1 |
| Slco1a4 | | Itga6 | Rnaseh2a |
| Selplg | | Ube4b | Msrb2 |
| Osmr | | Srm | Cd2bp2 |
| Ushbp1 | | Slco1c1 | Gfra4 |
| Eng | | Ltn1 | Commd7 |
| Tpp1 | | Matn2 | Arhgdig |
| Nudt2 | | Abcc6 | Srpr |
| Lrp5 | | Adgrf5 | Fam21 |
| Prom1 | | Myo10 | 2810001G20Rik |
| Ccl9 | | Actn4 | Gtf3c6 |
| Fn1 | | Dock4 | Nipsnap1 |
| Robo4 | | Fli1 | Prune |
| Itpr2 | | Itih5 | Erc1 |
| Tie1 | | Adgrl4 | Has1 |
| Shroom4 | | Cp | Cyb561 |
| | | Macf1 | 6030458C11Rik |
| | | Osbpl8 | Ebpl |
| | | Gnptab | B4galnt4 |
| | | Slfn5 | Wdr76 |
| | | Ano2 | Hcfc1r1 |
| | | Wdsub1 | Fmnl1 |
| | | Fam65a | Fam149b |
| | | Gstz1 | Rbm43 |
| | | Lrrc32 | Mmp23 |
| | | Slitrk2 | 2900011O08Rik |
| | | Inmt | Cdca7l |
| | | She | Magi1 |
| | | 2410018L13Rik | Slc37a3 |
| | | Tbc1d15 | Cacfd1 |
| | | Scarf1 | Fam173a |
| | | Fhod1 | Ap3s2 |
| | | Crybg3 | Uchl1 |
| | | Slc25a34 | Pkd1 |
| | | Spry2 | Cd59b |
| | | Gpr26 | Dnajc1 |
| | | Ttll3 | Endov |
| | | II16 | 0610037L13Rik |
| | | Tcaf1 | Mrc2 |
| | | Itga1 | Adi1 |
| | | Tnxb | Pop4 |
| | | Tet3 | Stoml1 |
| | | Midn | Zfp764 |
| | | Casc3 | Zfp358 |
| | | Atp1a1 | Rsrp1 |
| | | Hmcn1 | Agps |
| | | Arap3 | Plscr3 |
| | | Tmem204 | Rhbdd3 |
| | | Ppm1d | Slc35e4 |
| | | Hbegf | Oxnad1 |
| | | Sh3tc1 | Zfp580 |
| | | Eftud2 | Dph3 |
| | | Abcc4 | Dpy30 |
| | | Phc3 | Ltk |
| | | P4ha1 | Zfp811 |
| | | Setd1a | Upf3a |
| | | Plekhg2 | Adal |
| | | Homer1 | Cops8 |
| | | Slc26a10 | Cpt1a |
| | | Sema3g | Gnb1 |
| | | Hmgcr | Fam73a |
| | | Kcnh5 | Vegfa |
| | | Notch3 | Tmem42 |
| | | Kctd16 | Srp9 |
| | | Egr3 | Ap2s1 |
| | | Serinc3 | 1110051M20Rik |
| | | Arhgap32 | Zfp316 |
| | | Traip | 2210013O21Rik |
| | | Cntn5 | Aox3 |
| | | Abcg2 | Tmsb10 |
| | | Slco1a4 | Kiz |
| | | Znfx1 | Baalc |
| | | Pcgf5 | Rnf170 |
| | | Camsap1 | Kdr |
| | | Ece1 | B3gat3 |
| | | Farp1 | 2810032G03Rik |
| | | Kit | Mmab |
| | | 9030617O03Rik | Maf |
| | | Sipa1l3 | Flywch2 |
| | | Csmd1 | Dgcr6 |
| | | Hmha1 | Pus1 |
| | | Klf2 | 6430571L13Rik |
| | | Per2 | E2f2 |
| | | Tbx3 | Bloc1s5 |
| | | Nphp4 | Pole3 |
| | | Mfng | Dlat |
| | | Parp14 | Gas5 |
| | | Pcdhga11 | Tmub1 |
| | | Dcaf13 | Cxx1b |
| | | Hsp90b1 | Bcap29 |
| | | Tns1 | Hn1 |
| | | Klf4 | Cars |
| | | Epas1 | Pkia |
| | | Zc3h11a | Myl6 |
| | | Crlf3 | Tspan9 |
| | | Smg1 | Echs1 |
| | | Ccdc8 | Cecr5 |
| | | Irf9 | Fndc4 |
| | | Lama3 | Uqcrfs1 |
| | | Pdzd2 | 2610008E11Rik |
| | | Usp7 | Mpnd |
| | | Icam2 | Setmar |
| | | Mbnl2 | Bag1 |
| | | Baz1a | Cfdp1 |
| | | Atp2a2 | Ncaph |
| | | Egr2 | Chrnb4 |
| | | Cttnbp2 | Has3 |
| | | Rpl35a | Abhd14b |
| | | Myl9 | Snhg20 |
| | | Abcc9 | Praf2 |
| | | Gm10548 | Lgals4 |
| | | Pcdhga7 | Tomm40l |
| | | Vcl | Brpf1 |
| | | Lrrk2 | Mrpl13 |
| | | 6330403K07Rik | Mrps34 |
| | | Plxna2 | Atp13a2 |
| | | A930017M01Rik | Impa1 |
| | | Adap2 | Bgn |
| | | Ushbp1 | Mettl8 |
| | | Spred2 | Mturn |
| | | Amdp2 | Prdm15 |
| | | Mfap3 | Atg7 |
| | | Upf1 | Myof |
| | | Rreb1 | Timm8b |
| | | Gsdmd | Particl |
| | | Hip1 | Ryr2 |
| | | Chsy3 | Nmnat1 |
| | | Gga3 | Myh11 |
| | | D130040H23Rik | Ctbp1 |
| | | Ptpn14 | Tmem9b |
| | | Fcho2 | Gltscr1 |
| | | Eng | Rhbdf1 |
| | | Cmklr1 | Csf3r |
| | | Sik2 | 1700025G04Rik |
| | | Rasip1 | Exd1 |
| | | Cyp2d22 | Pus7 |
| | | Zkscan3 | |
| | | Tnfrsf1b | |
| | | Nudt2 | |
| | | Lhcgr | |
| | | Lrp5 | |
| | | Prom1 | |
| | | Filip1l | |
| | | Ets1 | |
| | | Uba7 | |
| | | Ptchd1 | |
| | | Irf1 | |
| | | Ephb4 | |
| | | Nes | |
| | | Zfp36 | |
| | | Sipa1 | |
| | | 5730508B09Rik | |
| | | Por | |
| | | Ripk2 | |
| | | AW549877 | |
| | | Cald1 | |
| | | Fn1 | |
| | | Gtf2h5 | |
| | | Vgf | |
| | | Robo4 | |
| | | Mmrn2 | |
| | | 6820408C15Rik | |
| | | Itpr2 | |
| | | Tie1 | |
| | | Cd93 | |
| | | Drosha | |
| | | Jag2 | |
| | | Bcdin3d | |
| | | Ranbp2 | |
| | | Sox13 | |
| | | Tarsl2 | |
| | | Layn | |
| | | Kbtbd2 | |
| | | Vps13d | |
| | | Map4k3 | |
| | | Shroom4 | |
| | | Gpr182 | |

TABLE 3
Differentiated gene expression (FDR <0.10)

| Rosa^{hAlfy/+} vs. WT | | Q140::Rosa^{hAlfy/+} vs. Q140 | | Q140::Rosa^{hAlfy/hAlfy} vs Q140 | |
|---|---|---|---|---|---|
| Down- | | Down- | | Down- | |
| regulated | Up-regulated | regulated | Up-regulated | regulated | Up-regulated |
| Robo4 | Sgsm2 | Gm4735 | Rpl22-ps1 | Gm4735 | Zfp992 |
| Phip | Cd47 | Crocc | Zfp992 | Crocc | Rpl22-ps1 |
| Gm8995 | RP24-229E19.1 | Arhgef5 | E230025N22Rik | Eno1 | Eno1b |
| Adgre5 | Hgsnat | Abcb1a | Eno1b | Miip | Cybrd1 |
| Adgrf5 | Rabep1 | Fbxo44 | Gm16399 | Zfp984 | Mthfr |
| Cdh5 | Sars2 | Slc2a5 | Gm20305 | Fbxo44 | RP23-179K7.3 |
| Eng | Dcp1b | Arhgef19 | Zfp990 | Slc2a5 | Setmar |
| Hspg2 | Blvrb | Gm12183 | Vwa7 | Gm6089 | Gm16399 |
| Slfn8 | Slc37a4 | Trak2 | Fxyd2 | Zfp933 | Clcn6 |
| Mmp28 | Lrrc49 | Miip | Apitd1 | Zfp982 | Ttc34 |
| Klf4 | RP23-179K7.3 | Robo4 | Wwox | Megf6 | Rabep1 |
| Shroom4 | Uchl1 | Eng | Gm8276 | Gm16503 | Sdhb |
| Mmrn2 | Plscr3 | Arhgap26 | Rgs22 | Arhgef19 | Erich5 |
| Flt1 | Glo1 | Eno1 | Slc37a4 | 4930481A15Rik | Gm20305 |
| Pcdhga7 | Fxyd2 | Ccdc141 | Sertad1 | Rsg1 | E230025N22Rik |
| Tie1 | Gm9008 | Megf6 | Xaf1 | Eng | Gm5855 |
| Btaf1 | Gm29718 | Cdh5 | AA465934 | Robo4 | Fam21 |
| Tjp1 | AA465934 | Flt1 | Fxyd6 | Ighg2c | Pla2g4e |
| Ppm1d | Pnmal1 | Cxcl12 | Pop4 | Adcy4 | Apitd1 |
| Notch4 | Psmd2 | Ceacam1 | Rps9 | Hnrnpa3 | Gid4 |
| Acaca | Gm11942 | P2ry13 | Clcn6 | Gm15631 | Gm8276 |
| Myole | Btbd9 | Zfp933 | Gm10036 | Cdh5 | Casp9 |
| Ryr1 | 2900052N01Rik | Polr2a | Use1 | Cecr5 | Pop4 |
| Gata2 | Gm10039 | Mbnl1 | Gm14295 | Sel1l | Tmcc1 |
| Abcb1a | Gm16092 | Mfsd7c | Gm14418 | Nos3 | Tprgl |
| Brip1os | Slc45a1 | Cdh2 | Unc45b | Nat8f5 | Rnf167 |
| Ptprb | Gm13443 | Itpr2 | Rps5 | Cxcl12 | Chrnb4 |
| Adcy4 | Pop4 | Zfp982 | Gm9625 | Gm5067 | Zfp386 |
| Igfbp7 | Nsun7 | Arhgap29 | Eif3f | Tie1 | Spop |
| Cxcl12 | Lrfn5 | Kdr | Hira | Flt1 | Smarcd3 |
| Pltp | Dlat | Tie1 | Rpl34-ps1 | Efcab12 | Rps9 |
| H2-T10 | Pfn1 | Slc25a34 | Erich5 | Arhgef5 | 4930563E22Rik |
| Abcc9 | Ndufb8 | Thsd1 | Sgsm2 | Itga6 | Wdsub1 |
| Txnip | Ntan 1 | Ube4a | Gm9008 | Cltb | Rwdd3 |
| Tpm4 | Asb1 | Slfn8 | Gm14403 | 4933413G19Rik | Gm6685 |
| Cobll1 | Nova1 | Fst14 | Tprgl | Setd5 | Nnt |
| Cyb5d2 | Wwox | Tnr | Al115009 | Bub1b | Xaf1 |
| Brd1 | Smim1012a | Rsg1 | Gid4 | Pear1 | Sgsm2 |
| Crybg3 | Med28 | Hcn1 | Rps19-ps3 | Zfp366 | Sumf1 |
| Ranbp2 | Gm5617 | Olfr287 | Rs124d1 | Pltp | Mdp1 |
| Ccm2l | Oaz2-ps | Hspb8 | Ppp1r32 | Dhx9 | Rps19-ps3 |
| Esam | Zfp990 | Gm8995 | Pithd1 | Mfap2 | Snhg8 |
| Plscr4 | Grid2ip | Mmp28 | Gm13123 | Lamc3 | Rhno1 |
| Med13 | Gm6685 | Slc22a8 | Utp18 | Gata2 | Gm2a |
| Zbtb11 | Nova2 | Ptprb | Pim3 | Hspg2 | Zfp286 |
| Cecr5 | Pnpo | Plscr4 | Lsp1 | Cntn4 | Zfp706 |
| Cyp51 | Pinx1 | Gcc2 | Gm16092 | Abcb1a | Zfp990 |
| 3222401L13Rik | Lage3 | Fn1 | Slc14a2 | Epha2 | Gzmk |
| Rnf125 | Syngr1 | Nos3 | Gm4332 | Fam173b | Gm12174 |
| Zfp366 | Setmar | Uaca | Nsa2 | Helz2 | Gm6257 |
| Tmem204 | Sae1 | Gm5454 | Gm15421 | Dock6 | Zfp106 |
| Megf6 | Gnl1 | Ccl9 | RP23-45713.2 | Ceacam1 | Stmn4 |
| Slc35a5 | Arhgap35 | Btaf1 | Mthfr | Birc6 | Adi1 |
| Trim47 | Eno2 | Prkdc | Rabep1 | Atxn2 | Coq2 |
| Adgrl4 | Eml2 | Adcy4 | Tmem179 | Lsr | Eif3j2 |
| Lpin3 | Pigb | Ccar1 | C1qtnf4 | Ccnd1 | Exd1 |
| Gm12183 | Pafah1b1 | Slco1a4 | Rpl7 | Ccm21 | 1110032A03Rik |
| Rasip1 | Xaf1 | Itih5 | N4bp3 | Eif4g1 | Med28 |
| Sox17 | Ccdc177 | Kank1 | Gm6733 | Rnf213 | Rps8 |
| Hist1h2be | Chd3os | Eif4g1 | Masp2 | Zgrf1 | Gm4366 |
| Cbl | Gm24339 | Pear1 | Chrnb4 | Ushbp1 | Eif3f |
| Scara3 | Timm17a | Esyt1 | Rnf167 | Adgre5 | Rpsa-ps10 |
| Itga1 | Ift57 | Tmem100 | Ap4s1 | Sox17 | Rpl4 |
| Decr2 | Gm266 | Selplg | 2900052N01Rik | Shroom4 | Mettl9 |
| Stard4 | Pde9a | Spin2c | Aim1l | Tjp1 | Rpusd4 |
| Oscar | Slc2a4 | Rab11fip1 | Cenpv | Esyt1 | Ica1l |
| 6330403K07Rik | Uhrf1bp1 | Ocln | Fbxo6 | Vps13a | Arhgdig |
| Ocln | | Shroom4 | Rpl4 | Cobll1 | Fxyd2 |
| Ppil1 | | Cecr5 | Gm10687 | Rimklb | Trmt5 |
| Ushbp1 | | Ptprg | Ptpa | Gm8995 | Rnf187 |
| Zfp109 | | Mamdc2 | RP24-229E19.1 | Fzd6 | Oraov1 |
| 4931406P16Rik | | Kcp | Smim7 | Ppfia1 | Use1 |
| Klf2 | | Srp72 | 4930563E22Rik | Tpcn2 | Nub1 |
| Upk1b | | Ubap21 | Spop | Mfsd7c | Hira |
| Helz2 | | Znf41-ps | Gm9899 | Ryr1 | Rab1b |
| Kank3 | | Cdc23 | Rpl8 | Pcdha12 | Nicn1 |
| Btg3 | | Pcdhb2 | Tekt1 | Itfg2 | Pithd1 |
| Hmcn1 | | Ubr4 | Stmn4 | Gm21168 | Rnf207 |
| Hyal1 | | Sec23b | Oaz2 | Brd1 | Arhgef15 |
| Atp10d | | Mut | Blvrb | Uaca | Rpl7 |
| Sel1l | | Alas2 | Rps8 | Nup210 | Chd3os |
| Rin3 | | Birc6 | Psd | Btaf1 | Rpl9-ps6 |
| Atp7a | | Ide | Zfp706 | Vps13d | 2410006H16Rik |
| Cpox | | Arid4a | Nxph3 | Znf41-ps | Rhov |
| Sema3g | | Lsr | Fgf5 | Eif4g3 | Gm2000 |
| Ehd2 | | Rb1cc1 | E2f2 | Aak1 | Fbxo2 |
| Arl13b | | Cp | Rpl18a | Ptprb | D430019H16Rik |
| Mfsd7c | | Il6ra | Gm12892 | Slco1a4 | RP24-229E19.1 |
| Itga6 | | B230209E15Rik | Shfm1 | Tmem88 | Eif5al3-ps |
| Pcdhga3 | | Hmha1 | Lrrc1 | Med13 | Ntan1 |
| Arhgap5 | | Tjp1 | Qdpr | Als2cl | Cyp2a5 |
| Pcdhga4 | | Efcab12 | Iqca | Dock9 | Slc2a4 |
| Cep97 | | Bbs7 | Gltscr2 | Hspa12b | Mettl8 |
| Tmem44 | | Notum | 9330151L19Rik | Kcp | Pkia |
| Urb1 | | Cpt1a | Srd5a1 | Klf4 | Gm9843 |
| Trp53bp2 | | Csf3r | Slc25a28 | Myo10 | Agps |
| Msn | | Prom1 | Gm8730 | Szrd1 | Pfn1 |
| Lztfl1 | | Trappc8 | Rps6kl1 | Thbd | Rsl24d1 |
| Gm9517 | | Thbd | Eif5al3-ps | 5730508B09Rik | Ankrd46 |
| Pear1 | | Rnf214 | Ttc34 | Cyyr1 | Gm11478 |
| Ccr5 | | Hspa8 | Atp9a | Fn1 | Gm43305 |
| Sgms1 | | Spred3 | Rps27rt | Aqr | Gm5617 |
| Fmo5 | | Tbx18 | Rps4x | Ehd2 | Rpl13a |
| Soat1 | | Gm13961 | Gm11942 | Slc52a3 | Gm6472 |
| Atp6v0c | | Dock9 | Atp5e | Ccar1 | Fis1 |
| Mfsd14a | | Brip1os | Snx6 | Slc38a6 | Atf4 |
| Pik3c2a | | Bcam | Rad51d | Foxf2 | Ube2l3 |
| Tns2 | | Pla2g4a | Rpl13a | Lrrk1 | Gm10687 |
| Serpind1 | | Fyco1 | Fancc | Kdr | Banf1 |
| Gm14569 | | Adgrf5 | Gm9843 | 6-Mar | Icmt |
| Ephb4 | | Prcp | B4galnt4 | 9930111J21Rik2 | Slc37a4 |
| Layn | | Sp100 | Kctd2 | Sec24d | Mmab |
| A230046K03Rik | | Acaca | Ddi2 | Ocln | Cd2bp2 |
| Tle3 | | Cyyr1 | Tmem107 | Dhx15 | Arl6ip4 |
| Slc2a1 | | Slc38a11 | Prr11 | Skiv212 | Smim7 |
| Prelp | | Slc40a1 | B3glct | Ylpm1 | Gabarap |
| Vsig2 | | Stab1 | Arhgdig | Atad2b | Gm14586 |
| Zfp263 | | Dcn | Kif23 | Cntn6 | Rpl23a-ps3 |
| Ccl9 | | Tlr3 | Brk1 | Lamb2 | Adra2b |
| Lactb | | Cyfip1 | Arhgef15 | Sox7 | Gm9385 |
| Serpinb9 | | Setd5 | Mett19 | Dmxl2 | Ttc7b |
| Zfp772 | | Serinc3 | Bri3 | Ddx4 | Timm22 |
| Nos3 | | Abca9 | Cebpb | Slc22a8 | Gm10073 |
| Mvd | | Spag1 | Sdhb | Sox18 | Trim44 |
| Gm42732 | | Lrrc9 | Nudt2 | Esam | Eef1g |
| Hmbs | | Dock4 | Cox4i1 | Cald1 | Gm5526 |
| Trim12a | | Nemf | Gm12174 | Oscar | Ptpa |
| Itgav | | Tek | 2210016L21Rik | Cd93 | Rps27rt |
| Tmod3 | | Foxc1 | Sars2 | Notch4 | Nudt2 |
| Wnk1 | | Ushbp1 | 9330111N05Rik | Gm26512 | Vwa7 |
| Ahnak | | Lrp5 | Snrpd2 | Spred3 | Ift20 |
| Slc7a5 | | Dhx9 | Manbal | Usp15 | BC048546 |
| Lims2 | | Cracr2a | | Adgrf5 | Ptpn7 |
| | | Cldn5 | | Ubr4 | Snhg20 |
| | | Fgfbp3 | | Trappc8 | Gm33651 |
| | | Cfh | | 1500035N22Rik | Praf2 |
| | | Slc6a13 | | Lrp5 | Chchd1 |
| | | Helz2 | | Plekhg2 | Ap3s2 |
| | | C1qb | | Grm7 | Gm20383 |
| | | Tpp1 | | Sec23b | Pole3 |
| | | Jam2 | | Ranbp2 | Sord |
| | | Zmynd8 | | Dennd5b | 9330151L19Rik |
| | | Gm44250 | | Macf1 | Mfap1a |
| | | Cep290 | | Gpr68 | Fahd2a |
| | | Sox18 | | Tmc4 | Rpl18a |
| | | Phactr2 | | Gfm1 | Snf8 |
| | | Trip11 | | Lama4 | Sepw1 |
| | | Zic3 | | Tdp1 | Gpr34 |
| | | Spa17 | | Gm14567 | Mrfap1 |
| | | Mks1 | | Polr2a | Gstz1 |
| | | Slc46a3 | | Samd91 | Hs3st4 |
| | | Mtor | | Prom1 | Hdgfrp3 |
| | | Med13 | | Myh11 | Gcsh |
| | | Dscaml1 | | Tnfrsf1b | Dcp1b |
| | | Fgd5 | | Shprh | AA465934 |
| | | Polk | | Itpr3 | Fbxo6 |
| | | Samd91 | | Gbp9 | Gpx1 |
| | | Dlg1 | | Nes | Tifa |
| | | Oasl2 | | Adgra2 | Shfm1 |
| | | Zfp984 | | Sf1 | Tmem186 |
| | | Top1 | | Tns2 | Pdpn |
| | | Lingo2 | | Copb1 | Shox2 |
| | | Gata2 | | Pclo | Jund |
| | | Pclo | | Kank1 | Rpl19-ps11 |
| | | Usp9x | | Rasip1 | Pea15a |
| | | Osmr | | Plxnd1 | Rab11b |
| | | Usp8 | | Gm38372 | Gm20056 |
| | | Sod3 | | Fli1 | Rpl8 |
| | | Fry | | Stab1 | 2810428115Rik |
| | | Rnf213 | | Notch1 | Srp14 |
| | | Asap3 | | Klf2 | Fdx1l |
| | | Mgp | | Layn | Matn2 |
| | | Txlng | | Actn4 | Keap1 |
| | | | | Zswim8 | Rpl27a |
| | | | | Gm10524 | Gltscr2 |
| | | | | Ephb4 | Rgs22 |
| | | | | Zdhhc5 | Trappc2l |
| | | | | Syvn1 | Rpl38-ps2 |
| | | | | Ahnak | Rps3a1 |
| | | | | Icam2 | Qdpr |
| | | | | Gm12183 | 2210016L21Rik |
| | | | | Etv5 | Gpatch4 |
| | | | | Ptrf | Hpcal4 |
| | | | | Oasl2 | 11-Mar |
| | | | | Mbnl1 | Mea1 |
| | | | | Ptprg | Foxm1 |
| | | | | Fam120a | Ube2q2 |
| | | | | Ltbp4 | Plekhj1 |
| | | | | Sp100 | Nod2 |
| | | | | Lama5 | Smim1012a |
| | | | | Dopey1 | Gm6136 |
| | | | | Vsig2 | Gm13054 |
| | | | | Sec24b | Pgk1 |
| | | | | Spag1 | Gm5113 |
| | | | | Mybpc1 | Gm8292 |
| | | | | Slc38a11 | Gm7536 |
| | | | | Gm14648 | Rps21 |
| | | | | 6330403K07Rik | 2610008E11Rik |
| | | | | Tnxb | Ssb |
| | | | | Gm9517 | RP23-45713.2 |
| | | | | Mamdc2 | Nsmf |
| | | | | Ncoa3 | Rbx1 |
| | | | | Ube4a | Ccdc115 |
| | | | | Taf3 | Gm4149 |
| | | | | Nemf | Hrk |
| | | | | Iqgap1 | Rpl31-ps8 |
| | | | | Top1 | Kcnn1 |
| | | | | Tpp2 | Rpl11 |
| | | | | Flt4 | Tmem87a |
| | | | | BC005561 | Oaz2 |
| | | | | Sec16a | Particl |
| | | | | Epas1 | 4933406C10Rik |
| | | | | Ep300 | 1700037H04Rik |
| | | | | Thsd1 | Trnp1 |
| | | | | Ubr2 | Eef1a1 |
| | | | | Ubap21 | 2900052N01Rik |
| | | | | Taf1 | Rpsa |
| | | | | Furin | Atp13a2 |
| | | | | Plscr4 | Zmat2 |
| | | | | Ccdc88c | Gm14295 |
| | | | | Cpq | Cript |
| | | | | Nap1l2 | Gm15500 |
| | | | | Srpr | Rpl10a-ps1 |
| | | | | Pla2g4b | Eef1b2 |
| | | | | Stard9 | RP23-328F3.4 |
| | | | | Pla2g4a | Mrpl13 |
| | | | | Smc3 | Atp1b3 |
| | | | | Podxl | Rnaseh2a |
| | | | | Foxq1 | 2810001G20Rik |
| | | | | Bcam | Bbc3 |
| | | | | Aox3 | Blvrb |
| | | | | RP23-385N1.1 | Arhgef4 |
| | | | | Acaca | Msrb2 |
| | | | | Slitrk2 | D130020L05Rik |
| | | | | Ppp1r12a | Rpl14 |
| | | | | Mmrn2 | Thap6 |
| | | | | Kmt2a | Cenpv |
| | | | | Pcbp1 | Gas5 |
| | | | | Degs2 | 2810468N07Rik |
| | | | | Mon2 | Tmsb4x |
| | | | | Thumpd3 | Kctd2 |
| | | | | Pglyrp1 | Jtb |
| | | | | Pom121 | Rpl34-ps1 |
| | | | | Mllt4 | Gfra4 |
| | | | | Ccdc141 | 6530403H02Rik |
| | | | | Ivd | Aim1l |
| | | | | Rusc2 | Pfdn5 |
| | | | | Mtr | Ndufaf3 |
| | | | | Pcm1 | Gm10275 |
| | | | | Ube4b | Gm14418 |
| | | | | RP23-74012.8 | Commd7 |
| | | | | Nfe212 | 9130401M01Rik |
| | | | | Arhgap29 | Bpgm |
| | | | | Trip12 | Rps3a2 |
| | | | | Srm | Fam213b |
| | | | | Brd4 | Ncaph2 |
| | | | | Mmp28 | Gnptab |
| | | | | Slco1c1 | Sars2 |
| | | | | R3hdm2 | Tmem41a |
| | | | | Pkn3 | Gm7332 |
| | | | | Konj6 | Coq5 |
| | | | | Synj1 | Rps5 |
| | | | | Cd59b | Smim13 |
| | | | | Tmf1 | Gm5963 |
| | | | | Snx14 | Nupr1 |
| | | | | Atxn21 | Rpl39-ps |
| | | | | Dhx57 | Ncs1 |
| | | | | Ide | Dcaf11 |
| | | | | Gramd1b | Gm14287 |
| | | | | Scaf4 | Unc45b |
| | | | | Raph1 | Ftl1 |
| | | | | Pdgfb | Mrpl30 |
| | | | | Bgn | Gm9294 |
| | | | | Crybg3 | Tk2 |
| | | | | Tbc1d22b | St3gal5 |
| | | | | Itih5 | Gtf3c6 |
| | | | | Gm29367 | Gm9794 |
| | | | | Arhgap26 | Wdr76 |
| | | | | Rere | Gm16089 |
| | | | | Lrch3 | Nipsnap1 |
| | | | | Adap2 | Eral1 |
| | | | | Ltn1 | Gm15772 |
| | | | | Stard4 | Gm9008 |
| | | | | Tap2 | Rpl35 |
| | | | | Notum | Cdk4 |
| | | | | Nup153 | Chil1 |
| | | | | Gm13331 | Nop10 |
| | | | | Cdc23 | Gm16092 |
| | | | | Ric1 | Chmp3 |
| | | | | Hic1 | Rack1 |
| | | | | Pthlh | Prune |
| | | | | Gm5446 | Gm13629 |
| | | | | Plod1 | Erc1 |
| | | | | Afap112 | Ddit3 |
| | | | | Abcc6 | Has1 |
| | | | | Fam222b | Rnd2 |
| | | | | Lgals4 | Nsa2 |
| | | | | Usp19 | Akt2-ps |
| | | | | Plxna2 | Pex12 |
| | | | | Fam179b | Ahcyl2 |
| | | | | Bpnt1 | Ubiad1 |
| | | | | Gm15542 | Mturn |
| | | | | Myh9 | Manbal |
| | | | | Gbp7 | Purg |
| | | | | Nupl1 | Thra |
| | | | | RP24-286J21.7 | Gatc |
| | | | | 4932438A13Rik | Nudc |
| | | | | Fcho2 | Mrpl48-ps |
| | | | | Pdcd6ip | Aes |
| | | | | She | Vegfb |
| | | | | Usp9x | Gm10036 |
| | | | | Piezo1 | Snrpn |
| | | | | Dock4 | Fxyd6 |
| | | | | Zbtb33 | Rps11 |
| | | | | 4931406P16Rik | Cdkl4 |
| | | | | Ninj2 | Pdzd4 |
| | | | | Inmt | Gm26645 |
| | | | | Hspb1 | Gm4332 |
| | | | | Sec24c | Smyd5 |
| | | | | Prrc2c | Cyb561 |
| | | | | Vit | E2f2 |
| | | | | Sf3a1 | Cdca7l |
| | | | | Rbl2 | Htatsf1 |
| | | | | Cdh2 | Uqcrfs1 |
| | | | | Tbx3os1 | Tmem138 |
| | | | | Myo1c | Cgref1 |
| | | | | Hcn1 | Zfp459 |
| | | | | Pdk1 | Pdrg1 |
| | | | | Tek | BC031181 |
| | | | | Gm44777 | Anapc13 |
| | | | | Grn | Blmh |
| | | | | Adgrl4 | Bsdc1 |
| | | | | Nlrc5 | Rpl36a-ps2 |
| | | | | Wdfy3 | Rps16-ps2 |
| | | | | Pde4dip | Rpl7-ps7 |
| | | | | Ppp5c | Cd44 |
| | | | | Scyl2 | Guk1 |
| | | | | Vps13c | Ddx49 |
| | | | | Rin3 | Tmem18 |
| | | | | Mecom | Pycr1 |
| | | | | Lhcgr | Atp5e |
| | | | | Zfp326 | Nmnat1 |
| | | | | Nipbl | Rps23-ps1 |
| | | | | Tbx3 | Gm13974 |
| | | | | Cp | Ap2s1 |
| | | | | Cpt1a | Prps2 |
| | | | | Tpra1 | 9330132A10Rik |
| | | | | Lemd3 | Tmem179 |
| | | | | Cthrc1 | Ebpl |
| | | | | Uba7 | Rpl14-ps1 |
| | | | | Dennd3 | Ncaph |
| | | | | Als2 | Slc45a1 |
| | | | | Cpsf6 | Ift46 |
| | | | | Osbpl8 | Svbp |
| | | | | Cbll1 | Stub1 |
| | | | | Fstl4 | 1500004A13Rik |
| | | | | Mtmr6 | B4galnt4 |
| | | | | Tbx1 | Ctbp1 |
| | | | | Ralgapa1 | Brk1 |
| | | | | Atp7a | Triap1 |
| | | | | Txnip | Ccdc174 |
| | | | | Papolg | Neurl1b |
| | | | | Alas1 | Fkbp8 |
| | | | | Dtx3l | 1110051M20Rik |
| | | | | Ccdc186 | Adra2c |
| | | | | Arhgef101 | Gm20507 |
| | | | | Cdh11 | Hcfc1r1 |
| | | | | Parp14 | Phactr3 |
| | | | | Dmxl1 | Commd8 |
| | | | | Trak2 | Mrpl54 |
| | | | | Cep350 | AW146154 |
| | | | | Itgav | Ncf1 |
| | | | | Foxc1 | Cox20 |
| | | | | Maf | Fam149b |
| | | | | Hectd2 | Aplf |
| | | | | Kmt2e | Clec5a |
| | | | | Nfkb2 | Ubox5 |
| | | | | D130040H23Rik | Serpinf1 |
| | | | | Gm7964 | S100a16 |
| | | | | Zic3 | Rbm43 |
| | | | | Etv4 | Tomm401 |
| | | | | Map1b | Uqcr10 |
| | | | | Rsad2 | Timm21 |
| | | | | Mtor | 2900011OP08Rik |
| | | | | Gbp5 | Glo1 |
| | | | | Ift122 | Rpap1 |
| | | | | Frrs1 | Snrnp27 |
| | | | | Zc3h13 | Fam73a |
| | | | | Sec1415 | Tekt1 |
| | | | | Cav1 | Ckmt1 |
| | | | | Mdn1 | Zbtb22 |
| | | | | Trrap | Gm11942 |
| | | | | Sipa112 | Zfp850 |
| | | | | Brwd3 | Aif1 |
| | | | | Gfpt1 | Tmem9b |
| | | | | Rrs1 | Prox1os |
| | | | | Smek1 | Cisd1 |
| | | | | Myl9 | Epn3 |
| | | | | Gm6768 | Ubxn8 |
| | | | | Tigar | Snrpd2 |
| | | | | Tgm2 | Rpsa-ps12 |
| | | | | Nat8f3 | Rida |
| | | | | Slfn5 | Dhrs3 |
| | | | | C4b | Epb4114aos |
| | | | | Dhx29 | Oxsr1 |
| | | | | Dusp4 | Magi1 |
| | | | | Ano2 | Necab2 |
| | | | | Pcsk1 | Slc37a3 |
| | | | | Herc2 | 1190005106Rik |
| | | | | Gm5454 | Cfdp1 |
| | | | | Alox8 | Rgs10 |
| | | | | Rock2 | Gm10288 |
| | | | | Pcdhb2 | Sh3bgrl3 |
| | | | | Herc1 | Cacfd1 |
| | | | | Spin2c | Polr2j |
| | | | | Dlx1as | Myl6 |
| | | | | Fam65a | Thap3 |
| | | | | Caskin2 | Rps3 |
| | | | | Lrrc32 | Fam136a |
| | | | | Cmklr1 | Gm3934 |
| | | | | Cttn | Fam173a |
| | | | | Glcci1 | Denr |
| | | | | Wfdc1 | Rpl17-ps10 |
| | | | | Abcg2 | Mydgf |
| | | | | Ankrd17 | Rps15a-ps7 |
| | | | | Parp10 | Ptpmt1 |
| | | | | Abcc4 | Aar2 |
| | | | | Soga3 | Wdr89 |
| | | | | Lrp1b | Ctnnbip1 |
| | | | | Nfasc | Gm6204 |
| | | | | Vwf | Rpl26 |
| | | | | Il16 | Mrps15 |
| | | | | Gm694 | Prdx3 |
| | | | | Arfgef2 | Gxylt1 |
| | | | | Pcdhgc5 | Slmo1 |
| | | | | Col4a5 | Eno2 |
| | | | | Emsy | Smdt1 |
| | | | | Ankrd12 | Rps26-ps1 |
| | | | | 2410018L13Rik | Rrp15 |
| | | | | Nrip2 | Rnf181 |
| | | | | Sec24a | Pdzph1 |
| | | | | Cltc | Oaz1-ps |
| | | | | Cherp | Rps12 |
| | | | | Col14a1 | Uchl1 |
| | | | | Tbc1d15 | Slc35b4 |
| | | | | Ncam2 | Zpr1 |
| | | | | Scarf1 | Gmeb1 |
| | | | | Irf1 | Gm15337 |
| | | | | Mitf | 6820408C15Rik |
| | | | | Itpr2 | Ndufa5 |
| | | | | Jag2 | Snhg5 |
| | | | | Tcaf1 | Lrrc61 |
| | | | | Srpk2 | Tmem158 |
| | | | | Smarca5 | Slc50a1 |
| | | | | Fhod1 | Gm9996 |
| | | | | 6030458C11Rik | 4930526A20Rik |
| | | | | Ddb1 | Ankmy2 |
| | | | | Ptpn23 | Rps4x |
| | | | | Plekhm2 | Nhp2l1 |
| | | | | Igtp | Gpatch11 |
| | | | | Pcdh20 | Rpl37rt |
| | | | | Crlf3 | Sh3bp1 |
| | | | | Slc25a34 | Minos1 |
| | | | | Tap1 | Heatr3 |
| | | | | Baz2b | Tsr2 |
| | | | | Fam135b | Plekhb2 |
| | | | | Spry2 | Mrpl27 |
| | | | | Eln | Cars |
| | | | | Gpr26 | Endov |
| | | | | Rasa1 | 0610037L13Rik |
| | | | | Edem3 | Bag1 |
| | | | | Mfn2 | Rps24 |
| | | | | Ago2 | Maf1 |
| | | | | Hyou1 | Rab26 |
| | | | | Rb1cc1 | Timm8b |
| | | | | Camsap1 | Rpl9-ps7 |
| | | | | Ttll3 | Bloc1s6 |
| | | | | B2m | D8Ertd738e |
| | | | | Hmcn1 | Rps6-ps4 |
| | | | | Gcnt1 | Clec12a |
| | | | | Gm3764 | Snn |
| | | | | Itga1 | Mrpl35 |
| | | | | Sgms1 | Tpt1-ps3 |
| | | | | Uba6 | Slc20a1 |
| | | | | Kbtbd2 | Cwc27 |
| | | | | Kmt2b | Rnf7 |
| | | | | Pnpla6 | 6720427107Rik |
| | | | | Smg9 | Utp18 |
| | | | | Myo9b | Sec11c |
| | | | | Vgf | Wrb |
| | | | | Irgm1 | Bcl2l2 |
| | | | | Ccdc66 | Alpk1 |
| | | | | Tet3 | Ndufa13 |
| | | | | Pcdhga4 | Eif5a |
| | | | | Baz1b | Stoml1 |
| | | | | Ablim1 | Tomm22 |
| | | | | Midn | Ovgp1 |
| | | | | Farp1 | Mrpl11 |
| | | | | Fosb | 0610012G03Rik |
| | | | | Casc3 | Coq9 |
| | | | | Pum1 | Thoc1 |
| | | | | Usp8 | Zfp764 |
| | | | | Tinagl1 | Zfp358 |
| | | | | Edn1 | Gng13 |
| | | | | Gm12411 | Cstad |
| | | | | Fmnl1 | Rad52 |
| | | | | Zkscan3 | Coa5 |
| | | | | Gm10184 | Mrpl52 |
| | | | | Pigm | Mgat4b |
| | | | | Gpr4 | 3110040N11Rik |
| | | | | Gtf3a | Fam110b |
| | | | | Senp5 | Ssbp4 |
| | | | | Mark3 | Gm4459 |
| | | | | Lrp1 | Rsrp1 |
| | | | | Fry | Aptx |
| | | | | Mmp23 | Gm3052 |
| | | | | Trim47 | Ngrn |
| | | | | Utp14b | Ripk2 |
| | | | | Stra6 | Gm13112 |
| | | | | Atp1a1 | Pcdhga11 |
| | | | | Faxc | Gsr |
| | | | | Slc8a1 | Alox5ap |
| | | | | Adgrl2 | Adal |
| | | | | Kctd16 | Syt3 |
| | | | | Hipk3 | Kcnj10 |
| | | | | RP23-23512.1 | Zfp2 |
| | | | | Apobec1 | Plscr3 |
| | | | | Kif21a | Mpnd |
| | | | | Arap3 | Lsp1 |
| | | | | Tmem204 | Bloc1s2 |
| | | | | Lats2 | Kptn |
| | | | | Stab2 | Sh3bgrl2 |
| | | | | Ppm1d | Rhbdd3 |
| | | | | Pikfyve | Ahcy |
| | | | | Bod1l | Atp5j2 |
| | | | | Gm45110 | Slc35e4 |
| | | | | Cnot1 | Tmem241 |
| | | | | 2810002D19Rik | Oxnad1 |
| | | | | Rhbdf1 | Nelfe |
| | | | | Hbegf | Sec22c |
| | | | | Yeats2 | B3galt1 |
| | | | | Arhgap31 | Txnl4b |
| | | | | Dhx8 | Zfp580 |
| | | | | Sh3tc1 | 1110012L19Rik |
| | | | | Mfap1b | Wwox |
| | | | | Eftud2 | Gna13 |
| | | | | Gpr182 | Srxn1 |
| | | | | Sox13 | Gm7094 |
| | | | | Pggt1b | Tacr1 |
| | | | | Phc3 | Dph3 |
| | | | | Gm11405 | Kxd1 |
| | | | | Ptchd1 | Zcwpw1 |
| | | | | H2-Q4 | Echs1 |
| | | | | A4galt | Spcs1 |
| | | | | Fam114a2 | Dpy30 |
| | | | | P4ha1 | Mppe1 |
| | | | | Atp6v1h | Ndufs4 |
| | | | | Herc3 | Rpl13a-ps1 |
| | | | | Taf2 | Fbxl4 |
| | | | | Setd1a | Mroh5 |
| | | | | Cul4b | Ltk |
| | | | | Msi2 | D130017N08Rik |
| | | | | Slitrk4 | 4-Mar |
| | | | | Homer1 | Zfp709 |
| | | | | Nrp1 | Ssr3 |
| | | | | Scn7a | Zfp811 |
| | | | | Ipo4 | Mmel1 |
| | | | | Ttc21b | Hnrnpdl |
| | | | | Cse1l | Upf3a |
| | | | | Myof | Cops8 |
| | | | | Slc26a10 | Mkrn2 |
| | | | | Xpo1 | 1110008F13Rik |
| | | | | Sema3g | Dennd5a |
| | | | | Kmt2c | Yars |
| | | | | Nat8 | Hmgn1 |
| | | | | Pkd1 | Mrps18c |
| | | | | Ptpn2 | 1700048020Rik |
| | | | | Pcdh10 | A930017M01Rik |
| | | | | Hmgcr | Nr2c2ap |
| | | | | Mgp | Bod1 |
| | | | | Kcnh5 | Gm9768 |
| | | | | BC068281 | Sephs2 |
| | | | | Nid2 | Psmd8 |
| | | | | Dpp4 | Bet1 |
| | | | | Herc6 | Impa1 |
| | | | | Gm28438 | Fbxo8 |
| | | | | Pkd2 | Cnbd2 |
| | | | | Notch3 | Nit2 |
| | | | | Slc12a7 | Ypel1 |
| | | | | Cfap54 | Abhd14b |
| | | | | Haus2 | Thg1l |
| | | | | Il6ra | Ubl5 |
| | | | | Clint1 | Gnb1 |
| | | | | Fam161a | Ndufb5 |
| | | | | Nav1 | Gtf2h5 |
| | | | | Stxbp5l | Chmp1a |
| | | | | Txlng | Cdh9 |
| | | | | Egr3 | Cox7c |
| | | | | Zfp36 | Bnip31 |
| | | | | Smtn | Terf2ip |
| | | | | Serinc3 | Mrpl36 |
| | | | | Crnkl1 | Slc1a4 |
| | | | | Arhgap32 | Neurl1a |
| | | | | Epg5 | Ramp3 |
| | | | | Traip | Mett125 |
| | | | | Hspa5 | Ugt8a |
| | | | | Hcfc1 | Alg5 |
| | | | | Tnpo1 | Gnl1 |
| | | | | Dnajc1 | Mgll |
| | | | | Tln1 | Fbxl12os |
| | | | | Cntn5 | Kcns2 |
| | | | | Polk | Tmem42 |
| | | | | Cr1l | Vipr2 |
| | | | | Znfx1 | Srp9 |
| | | | | Mrc2 | Mrps24 |
| | | | | ligp1 | Zfp954 |
| | | | | Slc19a3 | Rtfdc1 |
| | | | | Kat6a | Anapc16 |
| | | | | Pcgf5 | Foxo6 |
| | | | | Cry1 | Nat14 |
| | | | | Gm20559 | Zdhhc16 |
| | | | | Sos1 | Rps25 |
| | | | | Ece1 | Akain1 |
| | | | | Ggt1 | Frat1 |
| | | | | Rassf9 | Rps26 |
| | | | | Gm38077 | Cox4i1 |
| | | | | Map3k1 | C1qtnf4 |
| | | | | Ryr2 | B3galt6 |
| | | | | Fgd5 | Fmc1 |
| | | | | Pcdha11 | Higd1a |
| | | | | Ncor2 | Setd6 |
| | | | | Anxa2 | Rpl29 |
| | | | | Appbp2 | Inafm2 |
| | | | | Rnf31 | Tma7 |
| | | | | Gm6501 | Cfap74 |
| | | | | Sfpq | Ing5 |
| | | | | Herpud1 | 1810043H04Rik |
| | | | | Rnf111 | Spryd3 |
| | | | | Gldc | C1qbp |
| | | | | Ptchd4 | Bbip1 |
| | | | | Spen | Mrps17 |
| | | | | Tbc1d8b | Lipo3 |
| | | | | Dync2h1 | Rpl41 |
| | | | | Nap113 | 6330418K02Rik |
| | | | | Kit | Rpl37a |
| | | | | Pigg | Gpc1 |
| | | | | Slc9a3r2 | Cnih2 |
| | | | | Brpf1 | Sae1 |
| | | | | Atp10a | Cln6 |
| | | | | Szt2 | Stard10 |
| | | | | Usp7 | Cenpb |
| | | | | Dcbld2 | Arpin |
| | | | | Mpdz | B3gat3 |
| | | | | Nktr | Brd2 |
| | | | | Fndc3b | D2Wsu81e |
| | | | | Alms1 | Ctxn1 |
| | | | | Vwa5a | Rpl35a |
| | | | | B230209E15Rik | Mlph |
| | | | | Spg11 | 1110038B12Rik |
| | | | | 9030617003Rik | Mterf2 |
| | | | | Bmx | Zfp882 |
| | | | | Zfc3h1 | Mrpl48 |
| | | | | Helz | Ubxn11 |
| | | | | Gm44167 | Mpv17l2 |
| | | | | Acta2 | Commd6 |
| | | | | Klhl28 | Gm9892 |
| | | | | Sipa1l3 | Gm10548 |
| | | | | Map4k3 | Gm37940 |
| | | | | Soat1 | Svil |
| | | | | Ets1 | Zbtb48 |
| | | | | Abcf2 | Gm13410 |
| | | | | Gm13031 | Jph4 |
| | | | | Trim16 | C1d |
| | | | | Atrx | Shtn1 |
| | | | | A330074H02Rik | Zfp316 |
| | | | | Gbp2 | Med31 |
| | | | | Rer1 | Pno1 |
| | | | | Slc16a4 | RP23-441D24.1 |
| | | | | Cntn3 | Snhg4 |
| | | | | Ankhd1 | 2210013021Rik |
| | | | | Por | Tmsb10 |
| | | | | Csmd1 | Gm6556 |
| | | | | Dvl3 | Kiz |
| | | | | Anxa3 | Zfand2b |
| | | | | Ppig | Pars2 |
| | | | | Hmha1 | Timm13 |
| | | | | Tbx18 | Bin2 |
| | | | | Il10rb | Bcdin3d |
| | | | | Gsdmd | Camk1d |
| | | | | Fam126b | Rnf170 |
| | | | | Ano1 | Wdr13 |
| | | | | Daam2 | Mrps16 |
| | | | | Gltscr1 | Bri3 |
| | | | | Per2 | Rps6ka1 |
| | | | | Zfp971 | E430024P14Rik |
| | | | | Kmt2d | Shq1 |
| | | | | Ipo7 | Rps15 |
| | | | | Arhgap5 | Chmp4b |
| | | | | Nphp4 | Psd |
| | | | | Ccdc61 | Scnm1 |
| | | | | Ints8 | 2810032G03Rik |
| | | | | Trappc11 | Parp6 |
| | | | | Mfng | Rps14 |
| | | | | Atm | Cbr3 |
| | | | | Dcaf13 | Alkbh4 |
| | | | | Ythdf3 | Flywch2 |
| | | | | Hsp90b1 | A830039N20Rik |
| | | | | Asap1 | Fstl1 |
| | | | | Tns1 | Lcmt1 |
| | | | | Eogt | Rpl3-ps1 |
| | | | | Pex1 | 1600012H06Rik |
| | | | | Stk3 | BB031773 |
| | | | | Tmem131 | Dgcr6 |
| | | | | Crebbp | Rtel1 |
| | | | | Dst | Wdr73 |
| | | | | Zc3h11a | Sumo3 |
| | | | | Nup155 | Rps19-ps6 |
| | | | | Tbx2 | Pus1 |
| | | | | Slc16a12 | 6430571L13Rik |
| | | | | Phf21b | Cdh23 |
| | | | | Cspg4 | Anxa4 |
| | | | | 2410089E03Rik | Bloc1s5 |
| | | | | Dync1i2 | Atg12 |
| | | | | Gabra1 | Pcbp4 |
| | | | | Apc | Plppr1 |
| | | | | Sik2 | Stx1b |
| | | | | N4bp2l2 | Aip |
| | | | | Drosha | Dlat |
| | | | | Smg1 | Eif3s6-ps2 |
| | | | | Slc23a2 | Mrps28 |
| | | | | Lrrn1 | Ei24 |
| | | | | Sh3pxd2b | B3galnt2 |
| | | | | Erbb4 | Fbxo4 |
| | | | | Mid1 | Gm17396 |
| | | | | Wnt5b | Atg7 |
| | | | | Zscan26 | Mrpl41 |
| | | | | Trpc4 | Tmub1 |
| | | | | Prkdc | Cxx1b |
| | | | | Itsn2 | RP24-163K4.1 |
| | | | | Clip1 | Gm14303 |
| | | | | Akap6 | Rps6kl1 |
| | | | | Ccdc8 | Smim12 |
| | | | | Vegfa | Rps19bp1 |
| | | | | AW549877 | Xpa |
| | | | | Bcl9 | Gm10736 |
| | | | | Dennd4c | Gm17018 |
| | | | | Chordc1 | Bcap29 |
| | | | | Mettl7a1 | Slc9a8 |
| | | | | Irf9 | Txndc12 |
| | | | | Lama3 | Orai2 |
| | | | | Zfp318 | Mad212 |
| | | | | Atp13a3 | Mrps34 |
| | | | | Pdzd2 | Coq8a |
| | | | | Csf3r | Atp6v1f |
| | | | | Xlr3b | Rfxap |
| | | | | Igfbp2 | Hn1 |
| | | | | Plekhh2 | Lamtor4 |
| | | | | Has3 | Tom1l1 |
| | | | | Hsp25-ps1 | 4833413G10Rik |
| | | | | Gm9104 | A930017K11Rik |
| | | | | Kcnma1 | 1700025G04Rik |
| | | | | Ankrd52 | Isoc2b |
| | | | | Cnot3 | Tspan9 |
| | | | | Hip1 | Cradd |
| | | | | Zfyve1 | Fndc4 |
| | | | | Ddx46 | Adcy7 |
| | | | | Arfgef1 | Brix1 |
| | | | | Agrn | Mpdu1 |
| | | | | Ttc37 | Taf11 |
| | | | | Fat1 | Galm |
| | | | | Mbnl2 | Fbxl15 |
| | | | | Cct8 | Ppp1r1a |
| | | | | P2rx4 | Gm8430 |
| | | | | Baz1a | |
| | | | | Atp2a2 | |
| | | | | Prrx1 | |
| | | | | Slc6a13 | |
| | | | | A930004J17Rik | |
| | | | | Fgfbp3 | |
| | | | | Exph5 | |
| | | | | Rpl21 | |
| | | | | Pcdhac2 | |
| | | | | Ap2a1 | |
| | | | | Wnk1 | |
| | | | | Egr2 | |
| | | | | Top2b | |
| | | | | Smchd1 | |
| | | | | Atad2 | |
| | | | | Parp4 | |
| | | | | Cttnbp2 | |
| | | | | Csmd2 | |
| | | | | Tjap1 | |
| | | | | Vcpip1 | |
| | | | | Son | |
| | | | | Rgl3 | |
| | | | | Dlg1 | |
| | | | | Bdp1 | |
| | | | | Safb | |
| | | | | Lnpep | |
| | | | | Rprd2 | |
| | | | | Kitl | |
| | | | | Zfp109 | |
| | | | | Abcc9 | |
| | | | | Ighm | |
| | | | | Med12l | |
| | | | | Iws1 | |
| | | | | Cyb5d1 | |
| | | | | Pcdhga7 | |
| | | | | Ipo5 | |
| | | | | Rasal2 | |
| | | | | 4930578C19Rik | |
| | | | | Gm13111 | |
| | | | | Hnrnpm | |
| | | | | Tarsl2 | |
| | | | | Vcl | |
| | | | | Trmt1l | |
| | | | | Lrrk2 | |
| | | | | Gm38394 | |
| | | | | Dnm2 | |
| | | | | Chd8 | |
| | | | | Bbs7 | |
| | | | | Baalc | |
| | | | | Lmbrd2 | |
| | | | | Cyp2d22 | |
| | | | | Btbd7 | |
| | | | | Ccdc88a | |
| | | | | Gcc2 | |
| | | | | Prdm10 | |
| | | | | Unc80 | |
| | | | | Prdm15 | |
| | | | | Spata1 | |
| | | | | Filip1l | |
| | | | | Nhlrc2 | |
| | | | | Magee1 | |
| | | | | Cfap57 | |
| | | | | Ifit1 | |
| | | | | Srgn | |
| | | | | Sgms2 | |
| | | | | Slc12a4 | |
| | | | | Palm2 | |
| | | | | Kat6b | |
| | | | | Dlc1 | |
| | | | | Hdac7 | |
| | | | | Ptprs | |
| | | | | Med15 | |
| | | | | Kdm5c | |
| | | | | Nid1 | |
| | | | | Tcf4 | |
| | | | | Xpo5 | |
| | | | | Trim34a | |
| | | | | Arid2 | |
| | | | | Gabrb2 | |
| | | | | Slfn8 | |
| | | | | Sipa1 | |
| | | | | H2-Q6 | |
| | | | | Tmem63b | |
| | | | | Brip1os | |
| | | | | Ar15a | |
| | | | | Ube3a | |
| | | | | Trim12a | |
| | | | | Stt3b | |
| | | | | Ggt5 | |
| | | | | Camta2 | |
| | | | | Api5 | |
| | | | | Trip10 | |
| | | | | Phactr2 | |
| | | | | Ntn4 | |
| | | | | Ago3 | |
| | | | | Map4k2 | |
| | | | | Klf5 | |
| | | | | Ifitm2 | |
| | | | | Shc1 | |
| | | | | Phip | |
| | | | | Dnmt1 | |
| | | | | Spred2 | |
| | | | | Ampd2 | |
| | | | | Pus7 | |
| | | | | Gm43843 | |
| | | | | Gm43858 | |
| | | | | Tpr | |
| | | | | Jmjd1c | |
| | | | | Map3k19 | |
| | | | | Rbm27 | |
| | | | | Stip1 | |
| | | | | Syne3 | |
| | | | | Phf2 | |
| | | | | Ss18 | |
| | | | | Abcd2 | |
| | | | | A330017A19Rik | |
| | | | | Mfap3 | |
| | | | | Arhgef11 | |
| | | | | Wdfy2 | |
| | | | | Ap4e1 | |
| | | | | Ifi203 | |
| | | | | Usp4 | |
| | | | | Upf1 | |
| | | | | Stt3a | |
| | | | | Azin1 | |
| | | | | Numa1 | |
| | | | | Wasf3 | |
| | | | | Dcaf8 | |
| | | | | Asxl2 | |
| | | | | Hgs | |
| | | | | Rreb1 | |
| | | | | Pgm3 | |
| | | | | RP24-225A21.3 | |
| | | | | Mphosph8 | |
| | | | | Gm28941 | |
| | | | | Chsy3 | |
| | | | | Gga3 | |
| | | | | Zbtb43 | |
| | | | | A230046K03Rik | |
| | | | | Prpf8 | |
| | | | | RP23-239H14.1 | |
| | | | | 2810403A07Rik | |
| | | | | Asap3 | |
| | | | | Jam2 | |
| | | | | Pfkm | |
| | | | | Ptpn14 | |
| | | | | Usp53 | |

### Example 3

Using two independent genetic approaches, it was found that increasing levels of Alfy prevents the accumulation of aggregated protein in mouse models of Huntington's disease and synucleinopathy (modeling Lewy Body's Dementia, Parkinson's disease, and the like). The genetic approaches used are ectopic overexpression of Alfy, as well as the introduction of a single nucleic acid change that represents a coding variant of the gene that encodes Alfy, Wdfy3; and A to G mutation that encodes Iso3032Val change. Both changes lead to transcriptional and translational upregulation of Alfy. By increasing Alfy levels, aggregate-accumulation and the subsequent onset of disease outcomes, including neuroinflammatory changes and others associated with neural stress (reactive astrocytosis, reactive microgliosis, downregulation of neuronal markers such as FoxP1), cell death, and behavioral deficits, are prevented. It is also shown that TDP43 (using a TDP43 overexpression model) a proteinopathy found in the majority cases of ALS, was altered by Alfy and this decrease in aggregation diminishes behavioral dysfunction as well.

In one embodiment, in vivo, Alfy levels may be augmented using two approaches: CRISPR Cas9 mediated introduction of the A to G mutation; and delivery of an RNA sequence known as AS2.

### Example 3

Increasing AS2 levels increases both transcript and protein levels of Alfy (FIG. 14).

Thus, augmenting levels of expression of autophagy linked FYVE protein (Alfy) can combat adult onset neurodegenerative disease in diseases other than HD including synucleinopathy and polyglutamine diseases and diseases associated with tau tangles (mouse model P301 S), which are relevant for tauopathies such as AD.

WDFY3-AS2 sequence (this contains the full AS2 sequence):

(SEQ ID NO: 3)
CTACTGAGCCGGCCGCAGAAATTGCAGCCGCTCAGCTTCTACCCCCTCCTGCCTTTCCTTCCTCTTT
CCTTACTTCCTTCCCTTCCCTCGGCTTCCCGCTCTTGCCTCACTCTCAGCGGCTGCCTTCGCCCCTG
TCTGCAGACAGCGCCGCTGGATGCTCCCAGCTGGACTTCAACCCCACTCCTCTCAGTCCCTCTCCCC
ACTGCCTTCCAGACGCGCCTCTTCCCCGCCCCGCGCCCCTCTCTCCTCTCCCACCCCTGCCCCTCT
CCGCGGCGCTCACCCTCCTCAGTCCCAGTTTCTGAAAGGACTCAGCTGAGAAAGGACAACTGGGTT
CCGCTTTCCTTAACCCTACACCCTTTAGCTGGATGCTGTCAGAGGCGATGGAGAAACGCAAAGGCTA
CTAGACGCAACAATAGAATTACCATATTGTTTTTCCTGGTTTGAAAGGACCAGATGGAAAGAAACTGG
GCAAGCCAATGAAAGTCTCTGGGGATCATGGGAATTGAGTGTCTATGAAAACCATATTCCAGACAAG
AATATAGTCTAAGGACACAGCAAGAGGCAACATTTTGGAAGCAGAGGGCAACTCTCACTGGAAACCA
AATCTCCTGGCACCTTGATCTTGGATTTTACAGCCTCCAGAACTGATAGCTGGGAGGCATTACATAGT
CATGTGCGAGGTCAAAACTGAGTCACCACCACAACCAAGTTCCAGCTGGCAAGAAGAGGAAAAATAA
CACAGAGGAGTTATGACCAGTGTTGTAAGGATCTGGCCTAGAAATGGCATGCATCTCTTCCACTAATA
TCCTATTGGTAAAAATGTAGTCACATGGGCACACCACCTATCTGCATGGAAGGCTGGGAAATGTTGA
TCGGTAGGGCAGCCATATGTGTAGGAAGGAGAAAACAGAATTTGGTGGATACCTAGTAGTCTCTGCC
ATACCCTTGACCAGAAATTTTTCTTCTAAAGAATTTAATTCTAAAAGTTATAGACATTCCCAAAGATATA
CCTGCAAGCATCACAGCTTTGTGTATAATTGCAAAAGTTGAAATCAACATGAATGCCCAAAATAGTGA
ATTATTTAAACAGATTATTACCCATATTATGAAGAAATACTCTGCAACTCTTAAAAATAAATGTAATGTG
CCAGGCGCAATGGGATATGCCTGCAGGCCCAGCTACACGGGAGGCCAAGGCAGGAGGATCACTTG
AAATCAAGAGTTCAAGTCTAGCCTAGACAACATAGTGAGACTTGGTCTCTAAAAAAGAAAAAAAAAAA
TAAAACTGATGTAATGAATATATAATTACAACAAAAGAGGTCTAACACTATAGTAGAAAAGTTTGGGGC
ACTCATCCCATTCATAATTATTTTACATTTAAATTACTGTTCCTACTTAAGTGGTGAGCTTGGTAACCAT
TTTCATATCAGGTGATACTGCTGCAGTAATCATAGCTGTTTGAATCAGGAATAATCCCAATCACATTTT
CTCTTAATCGTTTATTATACTGGGCATTGGGGTTGCAAGATAATGCACAGTCCGAGCTCAATACATGT
CTTCATGTACCTGTATGTAAATCCCACTTTTATTCTAAAATTCTTTGAATATTTTGTTTCTTGGAACCCA
ATGATCCTAAGAAAATTATGTACTTTTACCTGAAAATTAAAACCAATATTTATAAAAAGTGTATATGCAG
TATGATACCACTTTTAAAAAAAGATAAATTTATAGAAAAAAGTTTATATGACTGACAACAATGTCTCTGA
GTGGTAGGATTACAGTTAATTTTTATTTTCTTCATTCTACTTATCTTTATTTTCTGAACTTTCTACAATGA
ACATGTTTTATTTTTACTTTTGTTAAAATAAATAACATTTAATTTGACGGAAAATTCTAAAAATCAACATT
TGGTAACTTAAATATTCATTGGCACATGATTCTGTGCCTGTTTCTGGGGGTCATTCCCATGAGAAGAG
CAGGTAGAACTTATGAGAATAGAGCAGTCGAAATATTATAGGTTTCATATTTTACGGCTTTATTGAAAA
CATTGATATATTGAATATTCATAAATTTTAATTTAACTTTCTTTTATAATCCTTAATGTGAGGCTAAGGTA
TAAGGAGAAAGGAGATTGGTAAGGAAGTGTGATACATATTGAATCAGGTGTCAAGGTACCATTTGTG
CCTGGATCTGACCAATAGACCAAGGTAAAAATCTCAAAGAATGAGACTTGTAATGAGAATGCCACAAA
CTTGAATACCTTATGCACAAAACACAAACATTGTTATGCATAATGTACATGAACCCTGAAACAAAGATA
ATGATTTGGACCACCCAACTGAAGTGGCATGTTATGTTTTTGGCATGAACAAAAGAAACAAGAGAGAA
AAAAATCAAAATAATTAAAATAAATTGTTAGAGAAAAGTTACTTTAAAAATAGCTATAACGCTTTGCAAT
TTCAGTAACAGTTCTGCCTCTGATGCAATGTAAAACATGTATTAGACATTCAGGCTCCACAATAAATTT
TAATGAGCACTACAAATTTAGAATATATAAAAATATCAGTAACCTATGAATTCATCAACATAACTCACTT
TTCAGTTAAGTTAGGCTGAGTTGAAATTCTACTAAGTTTTCCCATATTCTTGATAAAGGCTAAATTTGA
AATAATTTATTTAGATGACTAATTGGCAATTTGATAGTAGTTACATTTTATGTAACACTTGATATAATAA
CCCAAATTAATTAGCTCAATATTATCTTCTGTGGTTTATTGATCCAAAAGTACATATTTGTTATGTAAAC
TTATACTATGTAAACTTGAATTAGTTTTTTGCTTCCTTGAATTAATAAAGCCCATTAATGCAAATCTAAG
CCTTCATAGACTTTTCCAATCCTTAGTTAAATATTAAAAACTGAGTATTGTAGTTATTTGCTAGGTATAA
AAGGTGACTCAGAGTAGAGAGTAAGATGTCAGCAAATAGACAATAAGATGTCAGCAAAAGTGCCTCA
AATTTTAACTGCGTCAAGTTAAGGGCATTGTAAATACTGTAGTTTTATGTAGCTTGCAACTTCTACTGA
AATGAGTTTAATCATGTCTTTCACATAGCTGAAGTTTGTGTCAAGATTTAAGCTGATTTTCTCATTCTTA
TCAAAGTCTCACCTGGGGGTAGGGAGGGGTAGGCCTACCTCCCACCTCTATACTATCATTTTAGGAA
CAGACAGGCATAAATCTGTTCATATGGTAGAACACATGATGTTAGAGTATATTTTGTTGAATGCTATG
GAATATTAATATAATTAATTCTAAAATATCACCTAAAATATGTCAACTGAAGATCTGACTGTACTAAATA
TGAAAAATAAAGCAGCACATACTTTC

### Example 4

### Alfy Base Editing

### Methods

### Guide RNA Design

Oligonucleotides ccggaggtattcttgcggtggaac (SEQ ID NO:4) and aaacgttccaccgcaagaatacct (SEQ ID NO:5) encoding single guide RNA (AGGTATTCTTGCGGTGGAAC; SEQ ID NO:6) were phosphorylated and annealed in following reaction mix:
1 μl oligo 1(100 μM)
1 μl oligo 2 (100 μM)
1 μl 10× T4 ligation buffer (NEB)
6,5 μl ddH2O
0.5 μl T4 PNK (NEB)

Incubating at 37 degrees for 30 min, followed by incubation at 95 degrees for 5 min and cooling down to 25 degrees at the speed 5 degrees per minute.

Annealed oligos were cloned into pGL3-U6-sgRNA-PGK-puromycin vector (gift from Xingxu Huang, Addgene plasmid #51133) cut with Bsal-HFv2 restriction enzyme.

### Base Editing

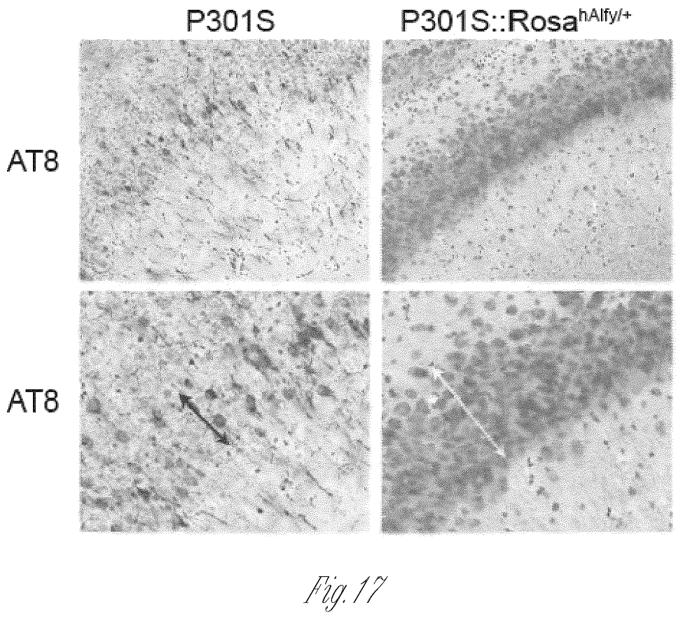

HEK293T cells were plated into 6 well plate at density 10⁵ cells/well and transfected 24 h later with prepared pGL3-U6-sgRNA-PGK-puromycin guide RNA vector and NG-ABE8e vector (gift from David Liu, Addgene plasmid #138491) in 1:1 ratio using X-tremeGENE 9 DNA Transfection Reagent (Roche). Complete media was supplemented with 2 μg/ml of puromycin 24 hours after transfection and cells were cultured in this media for additional 48 hours. 72 h after transfection (48 h after addition of puromycin) cells were lysed in 150 μl of DirectPCR Lysis Reagent (Cell) (Viagen Biotech), supplemented with 0.2 mg/ml of Proteinase K and incubated for 6 h at 55 degrees followed by incubation at 85 degrees for 45 min. 0.5 μl of cell lysate was used as a template to PCR region of genomic DNA around 13032 of ALFY/WDFY3 using primers pair Fw: CCACCCAGCAGGTCTTGTAG (SEQ ID NO:7) Rev: TGGCTAGGATCTCTCGGAGG (SEQ ID NO:8). Obtained PCR products were cloned into pCR-Blunt II-TOPO vector (Zero Blunt™ TOPO™ PCR Cloning Kit, Invitrogen) and transformed into Stbl3 chemically competent cells. Plasmid DNA was purified from 20 bacterial colonies and sequenced with T7 primer (TAATACGACTCACTATAGGG (SEQ ID NO:10)). Six out of 20 sequenced clones (30%) contained 13032V mutation (FIG. 16).

Thus, guide RNAs were identified that permit CRISPR mediated modification of WT Alfy to the variant.

### Example 5

P301S mice were crossed to mice overexpressing Alfy and assessed at 4 months of age. At this age, P301S mice show profound phospho-tau accumulation as shown by immunostaining against AT8 (brown), indicative of tau tangles (FIG. 17). Increasing levels of Alfy profoundly prevent the accumulation of tau. The lower image shows a higher magnification of the image from CA1 hippocampus that is shown about. Counterstaining using Nissl shows the degree of neuroprotection conferred by protecting against phosphor-tau accumulation. The black or white arrows are indicative of the width of the cellular layer CA1. Note how the presence of Alfy overexpression retains the number of CA1 cells. Similar protection is observed in cortex and amygdala (data not shown).

Therefore, increasing Alfy levels leads to a decrease in phospho-tau accumulation, as well as what appears to be a clear cytoprotective effect. Thus, Alfy overexpression is broadly protective across all major aggregates: polyglutamine, alpha-synuclein, TDP-43 and tau.

Antisense lncRNA WDFY3-AS2 with length of 3383 nucleotides which is located in chromosome 4q21.23. It immediately precedes the WDFY3 gene locus on the opposite strand. It is most highly expressed in the brain across all regions (NONCODE). AS2 has been speculated as a protective biomarker in several cancers. Given that IncRNAs can control gene expression via a ceRNA mechanism, AS2 could be acting as a ceRNAs to regulate the distribution of miRNA molecules on their targets and thereby impose an additional level of post-transcriptional regulation. WDFY3-AS2 is thought to be protective in multiple cancers as lower AS2 levels have been detected in cancerous tissue, and corrected when upregulated: In Esophageal Cancer (EC), AS2 acts through the miR-18a/PTEN Axis and was found to be a prognosis related lncRNA: correlated with survival and found to have low expression in EC patients (Li, 2020).. Furthermore, overexpression of WDFY3-AS2 repressed the progression of Esophageal Cancer by inhibiting cell proliferation, migration, and invasion. In Ovarian cancer, WDFY3-AS2 was found to be under-expressed in ovarian cancer with reduced WDFY3-AS2 expression in tumor tissue compared to adjacent normal tissue (Li, 2020). WDFY3-AS2 acts as a competing endogenous RNA to sponge miR-18a and upregulate RORA. Upon overexpressing WDFY3-AS2 or inhibiting miR-18a, RORA expression was increased, thereby the Ovarian cancer cell proliferation, migration, invasion, and epithelial-to-mesenchymal transition (EMT) were suppressed, accompanied by enhanced apoptosis. In Diffuse glioma, WDFY3-AS2, the top one of downregulated antisense IncRNAs in GBM with fold change of 0.441 (P<0.001) WDFY3-AS2 downregulation was closely correlated with tumor grade and poor prognosis in patients (Wu, 2018). In Oesophageal squamous cell carcinoma (Zhang, 2020) AS2 is regulating miR-2355-5p/SOCS2 axis. WDFY3-AS2 was down-regulated in ESCC tissues and cells, and its expression was correlated with TNM stage, lymph node metastasis and poor prognosis of ESCC patients. WDFY3-AS2 down-regulation significantly promoted cell proliferation and invasion, whereas WDFY3-AS2 up-regulation markedly suppressed cell proliferation and invasion in ESCC EC9706 and TE1 cells, coupled with EMT phenotype alterations. Lastly, in Breast cancer (Rodrigues, 2020; Deva, 2019) long non-coding WDFY3-AS2 RNA was identified as downregulated in breast tumors relative to normal tissue according to previous analyses in this study, low expression of this transcript was associated with worse prognosis in women with breast cancer. WDFY3-AS2 expression is associated with worse prognosis in breast cancer patients, including those classified as a basal or triple-negative subtype, suggesting that WDFY3-AS2 may act as a tumor suppressor gene for breast cancer. Lastly, we have shown that ALFY is essential for granulocytic differentiation of APL (acute promyelocytic leukemia) cells and that miR-181b caused a significant down-regulation of the basal ALFY mRNA levels in APL cells (Schlafli, 2017). Although AS2 levels have been found to be downregulated at the transcript level in cancers, and upregulation is potentially protective, no group has established if the observed effects is related entirely to the lncRNA, or if there is a component to the genes that AS2 controls. By demonstrating the work with Alfy's protection in neurodegenerative disorders, Alfy may be expanded into cancer treatment through the relationship with AS2.

### REFERENCES

Aber et al., Cell Rep., 41(3):111480 (2022).
Abernathy & Yoo, Cell Tissue Res., 359:179 (2015).
Abernathy et al., Cell Stem Cell., 21:332 (2017).
Abudu et al., Autophagy., 15(10):1845-1847 (2019).
Andrew et al., Nat. Genet., 4:398 (1993).
Banez-Coronel et al., Neuron, 88:667 (2015).
Christian et al., Am. Heart J., 164:846 (2012).
Cinesi et al., Nat. Commun., 7:13272 (2016).
Clausen et al., Autophagy, 6:330 (2010).
Cremona et al., Nat. Neurosci., 14:469 (2011).
Croce & Yamamoto, Neurobiol. Dis., (2018).
Deva Magendhra Rao A K, et al., Mol Oncol. 2019 Jun.;13(6):1342-1355.
DiFiglia et al., Proc. Natl. Acad. Sci. U.S.A., 104:17204 (2007).
Dragich et al., eLife, 5:e14810 (2016).
Dragich et al., Society for Neuroscience, San Diego, CA (2013).
Dukare & Klempnauer, Biochim. Biophys. Acta., 1859:914 (2016).
Eenjes et al., J. Cell Sci., 129(6): 12 6 0 (2016).
Ferrante, Biochim. Biophys. Acta., 1792:506 (2009).
Filimonenko et al., J. Cell Biol., 1793:485 (2008).
Filimonenko et al., Molecular Cell., 38:265 (2010).
Fox & Yamamoto, “Macroautophagy of aggregation-prone proteins in neurodegenerative disease” ed. Hayat, M. A. AUTOPHAGY: Cancer, Other Pathologies, Inflammation, Immunity, and Infection. Vol. 6. Academic Press, San Diego, CA., 2015
Fox et al., Neuron, 105(5):813-821.e6 (20
Gayan et al., Genet. Epidemiol., 32:445 (2008).
Gomes, C. M. Methods in Molecular Biology: Protein Misfolding Diseases. Springer Protocols. Springer Journals, 2018.
Gray et al., J. Neurosci., 28:6182 (2008).
Han et al., In Vitro Cell. Dev. Biol. Anim., 51:249 (2015).
Harper et al., Proc. Natl. Acad. Sci. U.S.A., 102:5820 (2005).
Harper, Huntington's Disease, (W.B. Saunders Company Ltd., London, U. K., 1996).
Hoji-Kawata et al., Nature, 494:201 (2013).
Itakura & Mizushima, Autophagy, 6:764 (2010).
Iwata et al., J. Biol. Chem., 280:40282 (2005b).
Iwata et al., Proc. Natl. Acad. Sci. U.S.A., 102:13135 (2005a).
Jeong et al., Cell, 137(1):60 (2009).
Johnson et al., Fut. Med. Chem., 4(13):1715 (2012).
Kirkin et al., Molecular Cell., 33:505 (2009).
Kirkin et al., Molecular Cell., 34:259 (2009).
Korac et al., J. Cell. Sci., 126:580 (2013).
Kuo et al., Acta Neuropathol., 125:879 (2013).
Lastres-Becker et al., Brain Res., 929(2):236 (2002).
Li W, et al., J. Cell. Physiol., 235:1141 (2020).
Lim & Yue, Dev. Cell., 32:491 (2015).
Liu et al., J. Cell. Biol., 216:1301 (2017).
Liu et al., Pathol. Oncol. Res., 26:1029 (2020).
Lystad et al., EMBO Rep., 15:557 (2014).
MacDonald & Gusella, Curr. Opin. Neurobiol., 6:638 (1996).
Machida et al., Biochem. Biophys. Res. Commun., 343:190 (2006).
Martin-Aparicio et al., J. Neurosci., 21(22):8772 (2001).
Menalled et al., J. Comp. Neurol., 465:11 (2003).
Menalled et al., PloS one, 7:e49838 (2012).
Mijaljica et al., Autophagy, 8:1701 (2012).
Napoli et al., Sci. Rep., 8:11348 (2018).
Nath et al., Nat. Cell. Biol., 16(5):415 (2014).
Odagiri et al., Acta. Neuropatholoqica, 124:173 (2012).
Pankiv et al., J. Biol. Chem., 282:24131 (2007).
Pizzo et al., Mol. Psych., 18(7):824 (2013).
Pre et al., Plos One. 9(7):e103418 (2014).
Ravikumar et al., Hum. Mol. Genet., 11:1107 (2002).
Ravikumar et al., Nat. Genet., 37:771 (2005).
Richner et al., Nature Protocols. 10:1543 (2015).
Richter et al., Proc. Natl. Acad. Sci. U.S.A., 113:4039 (2016).
Rodrigues de Bastos & Nagai, PLoS One. 15:e0232284 (2020).
Schilling et al., Hum. Mol. Genet., 8:397 (1999).
Schlafli A M, et al., Sci Rep. 7(1):12980 (2017).
Shoji-Kawata et al., Nature, 494:201 (2013).
Snyder-Keller et al., J. Neuropathol. Exp. Neurol., 69:1078 (2010).
Southwell et al., J. Neurosci., 29:13589 (2009).
Steffan, Cell. Cycle, 9:3401 (2010).
Stromhaug et al., Biochem. J., 335 (Pt 2):217 (1998).
Svane et al., J. Chem. Phys., 144:084708 (2016).
Tang et al., Neuron, 83(5):1131 (2014).
Thurston et al., Nature Immunology, 10:1215 (2009).
Vaquer-Alicea & Diamond, Annu. Rev. Biochem., 88:785 (2019).
Victor et al., Nat. Neurosci., 21:341 (2018).
Victor et al., Neuron, 84:311 (2014).
Vos et al., Hum. Mol. Genet., 19:4677 (2010).
Walker, Lancet., 369:218 (2007).
Wang et al., J. Cell. Biol., 181:803 (2008).
Weidberg & Elazar, Sci. Signal, 4:pe39 (2011).
Wexler et al., Proc. Natl. Acad. Sci. U.S.A., 101:3498 (2004).
Wild et al., Science, 333:228 (2011).
Wu, F., et al. Cancer Cell Int 18, 107 (2018).
Yamamoto & Yue, Ann. Rev. Neurosci., 37:55 (2014).
Yamamoto et al., Cell, 101:57 (2000).
Yamamoto et al., J. Cell. Biol., 172:719 (2006).
Yamamoto et al., Neurobiol. Dis., 8(6):923 (2001).
Yang-Klingler et al., Springer Protocols. Springer Journals, (2018).
Yoo et al., Nature, 476:228 (2011).
Yu et al., Autophagy., 14:207 (2018).
Yu et al., J. Neurosci., 23:2193 (2003).
Yuan et al., J. Biol. Chem., 285:28953 (2010).
Zhang et al., J. Cell Mol. Med., 24:8206 (2020).
Zheng et al., PLoS Genet., 6:e1000838 (2010).

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to prevent, inhibit or treat a proteinopathy in a mammal, comprising administering to the mammal a composition an effective amount of isolated nucleic acid comprising a nucleotide sequence encoding Alfy or a portion thereof, an Alfy-specific long non-coding RNA (LncRNA) or a corresponding DNA sequence, a vector comprising a nucleotide sequence encoding Alfy or a portion thereof, or isolated Alfy or a portion thereof.

2. The method of claim 1, wherein the mammal has or is at risk of having Huntington's disease, Parkinson's disease, Lou Gehring's disease, or a disease associated with an aberrant TDP43 or aberrant expression thereof.

3. The method of claim 1, wherein the Alfy or portion thereof has at least one amino acid substitution that if present in full length Alfy results in a variant Alfy that enhances clearance of protein aggregates relative to an Alfy without the one or more substitutions.

4. The method of claim 1, wherein the vector is a viral vector or a set of viral vectors.

5. The method of claim 4, wherein the set of viral vectors each comprises a different portion of the coding region for Alfy.

6. The method of claim 5, wherein each portion is linked to a N-terminal or C-terminal intein.

7. The method of claim 5, wherein each portion having a coding region is flanked by a splice acceptor site or a splice donor site, or both.

8. The method of claim 4, wherein the viral vector or set of vectors comprise adeno-associated virus, adenovirus, lentivirus or a herpesvirus.

9. The method of claim 1, wherein the composition comprises the LncRNA.

10. The method of claim 9, wherein a heterologous promoter is operably linked to DNA encoding Alfy or the portion thereof.

11. A method to prevent, inhibit or treat one or more symptoms of Alzheimer's disease, Amyotrophic Lateral Sclerosis, Frontotemporal dementia, parkinsonism-17, Frontotemporal lobar degeneration, Parkinson's disease, Huntington's disease, or Spinocerebellar ataxia type 3 in a mammal comprising: administering to the mammal a composition an effective amount of isolated nucleic acid encoding Alfy or a portion thereof, an Alfy-specific long non-coding RNA (LncRNA) or a corresponding DNA, a vector comprising a nucleotide sequence encoding Alfy or a portion thereof, or isolated Alfy or a portion thereof.

12. The method of claim 11, wherein the Alfy or portion thereof has at least one amino acid substitution that if present in full length Alfy results in a variant Alfy that enhances clearance of protein aggregates relative to an Alfy without the one or more substitutions.

13. The method of claim 11, wherein the vector is a viral vector or a set of viral vectors.

14. The method of claim 13, wherein the set of viral vectors each comprises a coding region for a portion of Alfy.

15. The method of claim 14, wherein each portion is linked to a N-terminal or C-terminal intein.

16. The method of claim 14, wherein each portion having a coding region is flanked by a splice acceptor site or a splice donor site.

17. The method of claim 13, wherein the viral vector or set of vectors comprise adeno-associated virus, adenovirus, lentivirus or a herpesvirus.

18. The method of claim 11, wherein the isolated nucleic acid comprises the LncRNA.

19. A method to prevent, inhibit or treat a neurodegenerative disease having protein aggregates in a human, comprising: administering to the human an effective amount of i) Cas or an isolated nucleic encoding Cas, and ii) isolated nucleic acid for one or more sgRNAs or pegRNAs comprising a targeting sequence for human Alfy genomic DNA.

20. The method of claim 19, wherein liposomes or nanoparticles comprise Cas or the isolated nucleic acid encoding Cas.

21. The method of claim 19, wherein liposomes or nanoparticles comprise the one or more sgRNAs or pegRNAs.

22. The method of claim 19, wherein a viral vector comprises the isolated nucleic acid encoding Cas.

23. The method of claim 19, wherein a viral vector comprises the one or more sgRNAs or pegRNAs.

24. The method of claim 19, wherein the one or more sgRNAs or pegRNAs comprise SEQ ID NO:6 or a nucleic acid sequence having at least 80% or 90% nucleic acid sequence identity thereto or a nucleic acid sequence having 1, 2, 3, 4, 5 or 6 nucleotide substitutions relative to SEQ ID NO:6.

25. A composition comprising isolated nucleic acid for one or more sgRNAs or pegRNAs comprising a targeting sequence for human Alfy genomic DNA and Cas or an isolated nucleic acid sequence encoding Cas.

26. The composition of claim 25, wherein the one or more sgRNAs or pegRNAs comprise SEQ ID NO:6 or a nucleic acid sequence having at least 80% or 90% nucleic acid sequence identity thereto or a nucleic acid sequence having 1, 2, 3, 4, 5 or 6 nucleotide substitutions relative to SEQ ID NO:6.

27. The composition of claim 25, wherein the targeting sequence includes a nucleotide sequence having at least one amino acid substitution at a position from 3025 to 3037 in human Alfy.

28. The composition of claim 27, wherein position 3032 has a valine.


SEQUENCE LISTING (27 sequences)

SEQ ID NO: 1 - protein, 3526 aa, Homo sapiens
MNMVKRIMGRPRQEECSPQDNALGLMHLRRLFTELCHPPRHMTQKEQEEKLYMMLPVFNRVFGNAPPNTMTEKFSDLLQFTTQVSRLMVTEIRRRASNKSTEAASRAIVQFLEINQSEEASRGWMLLTTINLLASSGQKTVDCMTTMSVPSTLVKCLYLFFDLPHVPEAVGGAQNELPLAERRGLLQKVFVQILVKLCSFVSPAEELAQKDDLQLLFSAITSWCPPYNLPWRKSAGEVLMTISRHGLSVNVVKYIHEKECLSTCVQNMQQSDDLSPLEIVEMFAGLSCFLKDSSDVSQTLLDDFRIWQGYNFLCDLLLRLEQAKEAESKDALKDLVNLITSLTTYGVSELKPAGITTGAPFLLPGFAVPQPAGKGHSVRNVQAFAVLQNAFLKAKTSFLAQIILDAITNIYMADNANYFILESQHTLSQFAEKISKLPEVQNKYFEMLEFVVFSLNYIPCKELISVSILLKSSSSYHCSIIAMKTLLKFTRHDYIFKDVFREVGLLEVMVNLLHKYAALLKDPTQALNEQGDSRNNSSVEDQKHLALLVMETLTVLLQGSNTNAGIFREFGGARCAHNIVKYPQCRQHALMTIQQLVLSPNGDDDMGTLLGLMHSAPPTELQLKTDILRALLSVLRESHRSRTVFRKVGGFVYITSLLVAMERSLSCPPKNGWEKVNQNQVFELLHTVFCTLTAAMRYEPANSHFFKTEIQYEKLADAVRFLGCFSDLRKISAMNVFPSNTQPFQRLLEEDVISIESVSPTLRHCSKLFIYLYKVATDSFDSRAEQIPPCLTSESSLPSPWGTPALSRKRHAYHSVSTPPVYPPKNVADLKLHVTTSSLQSSDAVIIHPGAMLAMLDLLASVGSVTQPEHALDLQLAVANILQSLVHTERNQQVMCEAGLHARLLQRCSAALADEDHSLHPPLQRMFERLASQALEPMVLREFLRLASPLNCGAWDKKLLKQYRVHKPSSLSYEPEMRSSMITSLEGLGTDNVFSLHEDNHYRISKSLVKSAEGSTVPLTRVKCLVSMTTPHDIRLHGSSVTPAFVEFDTSLEGFGCLFLPSLAPHNAPTNNTVTTGLIDGAVVSGIGSGERFFPPPSGLSYSSWFCIEHFSSPPNNHPVRLLTVVRRANSSEQHYVCLAIVLSAKDRSLIVSTKEELLQNYVDDFSEESSFYEILPCCARFRCGELIIEGQWHHLVLVMSKGMLKNSTAALYIDGQLVNTVKLHYVHSTPGGSGSANPPVVSTVYAYIGTPPAQRQIASLVWRLGPTHFLEEVLPSSNVTTIYELGPNYVGSFQAVCMPCKDAKSEGVVPSPVSLVPEEKVSFGLYALSVSSLTVARIRKVYNKLDSKAIAKQLGISSHENATPVKLIHNSAGHLNGSARTIGAALIGYLGVRTFVPKPVATTLQYVGGAAAILGLVAMASDVEGLYAAVKALVCVVKSNPLASKEMERIKGYQLLAMLLKKKRSLLNSHILHLTFSLVGTVDSGHETSIIPNSTAFQDLLCDFEVWLHAPYELHLSLFEHFIELLTESSEASKNAKLMREFQLIPKLLLTLRDMSLSQPTIAAISNVLSFLLQGFPSSNDLLRFGQFISSTLPTFAVCEKFVVMEINNEEKLDTGTEEEFGGLVSANLILLRNRLLDILLKLIYTSKEKTSINLQACEELVKTLGFDWIMMFMEEHLHSTTVTAAMRILVVLLSNQSILIKFKEGLSGGGWLEQTDSVLTNKIGTVLGFNVGRSAGGRSTVREINRDACHFPGFPVLQSFLPKHTNVPALYFLLMALFLQQPVSELPENLQVSVPVISCRSKQGCQFDLDSIWTFIFGVPASSGTVVSSIHNVCTEAVFLLLGMLRSMLTSPWQSEEEGSWLREYPVTLMQFFRYLYHNVPDLASMWMSPDFLCALAATVFPFNIRPYSEMVTDLDDEVGSPAEEFKAFAADTGMNRSQSEYCNVGTKTYLTNHPAKKFVFDFMRVLIIDNLCLTPASKQTPLIDLLLEASPERSTRTQQKEFQTYILDSVMDHLLAADVLLGEDASLPITSGGSYQVLVNNVFYFTQRVVDKLWQGMFNKESKLLIDFIIQLIAQSKRRSQGLSLDAVYHCLNRTILYQFSRAHKTVPQQVALLDSLRVLTVNRNLILGPGNHDQEFISCLAHCLINLHVGSNVDGFGLEAEARMTTWHIMIPSDIEPDGSYSQDISEGRQLLIKAVNRVWTELIHSKKQVLEELFKVTLPVNERGHVDIATARPLIEEAALKCWQNHLAHEKKCISRGEALAPTTQSKLSRVSSGFGLSKLTGSRRNRKESGLNKHSLSTQEISQWMFTHIAVVRDLVDTQYKEYQERQQNALKYVTEEWCQIECELLRERGLWGPPIGSHLDKWMLEMTEGPCRMRKKMVRNDMFYNHYPYVPETEQETNVASEIPSKQPETPDDIPQKKPARYRRAVSYDSKEYYMRLASGNPAIVQDAIVESSEGEAAQQEPEHGEDTIAKVKGLVKPPLKRSRSAPDGGDEENQEQLQDQIAEGSSIEEEEKTDNATLLRLLEEGEKIQHMYRCARVQGLDTSEGLLLFGKEHFYVIDGFTMTATREIRDIETLPPNMHEPIIPRGARQGPSQLKRTCSIFAYEDIKEVHKRRYLLQPIAVEVFSGDGRNYLLAFQKGIRNKVYQRFLAVVPSLTDSSESVSGQRPNTSVEQGSGLLSTLVGEKSVTQRWERGEISNFQYLMHLNTLAGRSYNDLMQYPVFPWILADYDSEEVDLTNPKTFRNLAKPMGAQTDERLAQYKKRYKDWEDPNGETPAYHYGTHYSSAMIVASYLVRMEPFTQIFLRLQGGHFDLADRMFHSVREAWYSASKHNMADVKELIPEFFYLPEFLFNSNNFDLGCKQNGTKLGDVILPPWAKGDPREFIRVHREALECDYVSAHLHEWIDLIFGYKQQGPAAVEAVNVFHHLFYEGQVDIYNINDPLKETATIGFINNFGQIPKQLFKKPHPPKRVRSRLNGDNAGISVLPGSTSDKIFFHHLDNLRPSLTPVKELKEPVGQIVCTDKGILAVEQNKVLIPPTWNKTFAWGYADLSCRLGTYESDKAMTVYECLSEWGQILCAICPNPKLVITGGTSTVVCVWEMGTSKEKAKTVTLKQALLGHTDTVTCATASLAYHIIVSGSRDRTCIIWDLNKLSFLTQLRGHRAPVSALCINELTGDIVSCAGTYIHVWSINGNPIVSVNTFTGRSQQIICCCMSEMNEWDTQNVIVTGHSDGVVRFWRMEFLQVPETPAPEPAEVLEMQEDCPEAQIGQEAQDEDSSDSEADEQSISQDPKDTPSQPSSTSHRPRAASCRATAAWCTDSGSDDSRRWSDQLSLDEKDGFIFVNYSEGQTRAHLQGPLSHPHPNPIEVRNYSRLKPGYRWERQLVFRSKLTMHTAFDRKDNAHPAEVTALGISKDHSRILVGDSRGRVFSWSVSDQPGRSAADHWVKDEGGDSCSGCSVRFSLTERRHHCRNCGQLFCQKCSRFQSEIKRLKISSPVRVCQNCYYNLQHERGSEDGPRNC

SEQ ID NO: 2 - protein, 3526 aa, Homo sapiens; identical to SEQ ID NO: 1

SEQ ID NO: 3 - DNA, 3383 bp, Homo sapiens
  source 1..3383: mol_type = other DNA
ctactgagccggccgcagaaattgcagccgctcagcttctaccccctcctgcctttccttcctctttccttacttccttcccttccctcggcttcccgctcttgcctcactctcagcggctgccttcgcccctgtctgcagacagcgccgctggatgctcccagctggacttcaaccccactcctctcagtccctctccccactgccttccagacgcgcctcttccccgccccgcgcccctctctcctctcccacccctgcccctctccgcggcgctcaccctcctcagtcccagtttctgaaaggactcagctgagaaaggacaactgggttccgctttccttaaccctacaccctttagctggatgctgtcagaggcgatggagaaacgcaaaggctactagacgcaacaatagaattaccatattgtttttcctggtttgaaaggaccagatggaaagaaactgggcaagccaatgaaagtctctggggatcatgggaattgagtgtctatgaaaaccatattccagacaagaatatagtctaaggacacagcaagaggcaacattttggaagcagagggcaactctcactggaaaccaaatctcctggcaccttgatcttggattttacagcctccagaactgatagctgggaggcattacatagtcatgtgcgaggtcaaaactgagtcaccaccacaaccaagttccagctggcaagaagaggaaaaataacacagaggagttatgaccagtgttgtaaggatctggcctagaaatggcatgcatctcttccactaatatcctattggtaaaaatgtagtcacatgggcacaccacctatctgcatggaaggctgggaaatgttgatcggtagggcagccatatgtgtaggaaggagaaaacagaatttggtggatacctagtagtctctgccatacccttgaccagaaatttttcttctaaagaatttaattctaaaagttatagacattcccaaagatatacctgcaagcatcacagctttgtgtataattgcaaaagttgaaatcaacatgaatgcccaaaatagtgaattatttaaacagattattacccatattatgaagaaatactctgcaactcttaaaaataaatgtaatgtgccaggcgcaatgggatatgcctgcaggcccagctacacgggaggccaaggcaggaggatcacttgaaatcaagagttcaagtctagcctagacaacatagtgagacttggtctctaaaaaagaaaaaaaaaaataaaactgatgtaatgaatatataattacaacaaaagaggtctaacactatagtagaaaagtttggggcactcatcccattcataattattttacatttaaattactgttcctacttaagtggtgagcttggtaaccattttcatatcaggtgatactgctgcagtaatcatagctgtttgaatcaggaataatcccaatcacattttctcttaatcgtttattatactgggcattggggttgcaagataatgcacagtccgagctcaatacatgtcttcatgtacctgtatgtaaatcccacttttattctaaaattctttgaatattttgtttcttggaacccaatgatcctaagaaaattatgtacttttacctgaaaattaaaaccaatatttataaaaagtgtatatgcagtatgataccacttttaaaaaaagataaatttatagaaaaaagtttatatgactgacaacaatgtctctgagtggtaggattacagttaatttttattttcttcattctacttatctttattttctgaactttctacaatgaacatgttttatttttacttttgttaaaataaataacatttaatttgacggaaaattctaaaaatcaacatttggtaacttaaatattcattggcacatgattctgtgcctgtttctgggggtcattcccatgagaagagcaggtagaacttatgagaatagagcagtcgaaatattataggtttcatattttacggctttattgaaaacattgatatattgaatattcataaattttaatttaactttcttttataatccttaatgtgaggctaaggtataaggagaaaggagattggtaaggaagtgtgatacatattgaatcaggtgtcaaggtaccatttgtgcctggatctgaccaatagaccaaggtaaaaatctcaaagaatgagacttgtaatgagaatgccacaaacttgaataccttatgcacaaaacacaaacattgttatgcataatgtacatgaaccctgaaacaaagataatgatttggaccacccaactgaagtggcatgttatgtttttggcatgaacaaaagaaacaagagagaaaaaaatcaaaataattaaaataaattgttagagaaaagttactttaaaaatagctataacgctttgcaatttcagtaacagttctgcctctgatgcaatgtaaaacatgtattagacattcaggctccacaataaattttaatgagcactacaaatttagaatatataaaaatatcagtaacctatgaattcatcaacataactcacttttcagttaagttaggctgagttgaaattctactaagttttcccatattcttgataaaggctaaatttgaaataatttatttagatgactaattggcaatttgatagtagttacattttatgtaacacttgatataataacccaaattaattagctcaatattatcttctgtggtttattgatccaaaagtacatatttgttatgtaaacttatactatgtaaacttgaattagttttttgcttccttgaattaataaagcccattaatgcaaatctaagccttcatagacttttccaatccttagttaaatattaaaaactgagtattgtagttatttgctaggtataaaaggtgactcagagtagagagtaagatgtcagcaaatagacaataagatgtcagcaaaagtgcctcaaattttaactgcgtcaagttaagggcattgtaaatactgtagttttatgtagcttgcaacttctactgaaatgagtttaatcatgtctttcacatagctgaagtttgtgtcaagatttaagctgattttctcattcttatcaaagtctcacctgggggtagggaggggtaggcctacctcccacctctatactatcattttaggaacagacaggcataaatctgttcatatggtagaacacatgatgttagagtatattttgttgaatgctatggaatattaatataattaattctaaaatatcacctaaaatatgtcaactgaagatctgactgtactaaatatgaaaaataaagcagcacatactttc

SEQ ID NO: 4 - DNA, 24 bp, synthetic construct
  source 1..24: mol_type = other DNA
ccggaggtattcttgcggtggaac

SEQ ID NO: 5 - DNA, 24 bp, synthetic construct
  source 1..24: mol_type = other DNA
aaacgttccaccgcaagaatacct

SEQ ID NO: 6 - DNA, 20 bp, synthetic construct
  source 1..20: mol_type = other DNA
aggtattcttgcggtggaac

SEQ ID NO: 7 - DNA, 20 bp, synthetic construct
  source 1..20: mol_type = other DNA
ccacccagcaggtcttgtag

SEQ ID NO: 8 - DNA, 20 bp, synthetic construct
  source 1..20: mol_type = other DNA
tggctaggatctctcggagg

SEQ ID NO: 9 - RNA, 14520 nt, Homo sapiens
  source 1..14520: mol_type = mRNA
agacagccagcgggaggtggagaaagcaggaggaggaggaggattaaagatggccaccaacagctgcgggaaacggcaacaacccctcactttccgggatggtccctgcgggtcggcccggccttgatggagagaagaaacccgaggagcgccgaggctgaggcggcggcggcggggacccagcgaggacgaggacgcggcggagcagggacgggggcaggagaagggaaaggcggcggcgtcgctgcccctgctgcctagcaccgctgcctggcccggcggaccggttcccatacctcgcggccgcagaatcgagctcgggccccggcccccggcccgcggcgcggggctcccgggccccgccgcggacgtcgcgccggtcgccccttccccgtagcccgtgcgccctcggcgcggagccccggcccgccgcggtcccgtctcctgggcctgtcccgcccgcgccctccgccggccctcaggtataatacttctccacgtctgcttcaggaagaaagtgcctgccattcttatcatttctaagcaggttcatgccagcccagaacagagaatcagctggagcccagatttcaagttttgagtaaaataccttcaagcgaatgggccctattgtgctcacacattcagaacctgttacccaaggaattccctaaagaattagaagtgcgtctcaccaaccagccaagatgaacatggtgaagaggatcatggggcggccgaggcaggaggagtgcagcccacaagacaacgccttaggactgatgcacctccgccggctcttcacggagttgtgccatcctccccggcacatgactcagaaggaacaagaagagaaactgtatatgatgctgccagtgtttaacagggtttttggaaatgctccgccgaatacaatgacagaaaaattttctgatcttctgcagttcacaacacaagtctcacgactaatggtgacagaaattcgaaggagagcatcaaacaaatccacagaggctgcaagtcgggccatagttcagttcctagagattaatcagagtgaagaagccagtagaggctggatgcttctaacgacaattaatttgttagcttcctctggtcagaaaaccgtggactgcatgacaacaatgtcagtgccttccaccctggttaaatgtttatatctgttttttgaccttccacatgtgcctgaggcagttggaggtgcacagaatgagctacctctagcagaacgtcgaggactactccagaaagtttttgtacagatcttagtgaaactgtgcagttttgtttcccctgcggaggagctggctcagaaagatgatctccagcttctattcagtgcaataacctcttggtgccctccctataacctgccttggagaaagagtgctggagaagtcctcatgaccatatctcgtcatggtcttagtgtcaatgtagtgaagtatattcatgagaaagagtgtttatctacatgtgttcagaatatgcagcaatcagatgacctgtctcccctagaaattgtcgaaatgtttgctgggctttcttgtttcctcaaagattccagcgatgtttcccaaacacttctggatgattttcggatatggcaaggatataattttctttgtgatctcttgcttagattggaacaagcaaaagaggcagaatccaaagatgccttgaaagatctggttaatctgataacttccctaacaacatatggtgtcagtgaactaaaaccagctggtattaccacaggggcaccctttttattgcctggatttgcagtacctcagcctgcaggcaaaggtcacagtgtgagaaacgtccaggcctttgcagttcttcagaatgcatttttaaaagcaaaaaccagcttccttgcccaaatcatccttgatgctatcacaaatatttacatggctgacaatgccaattacttcatcctagagtcacagcacacattgtcacagtttgcagagaagatttctaaactcccagaagtacaaaacaaatactttgagatgctggagtttgttgtttttagcttaaattatataccttgtaaagaacttattagtgtcagtatcctcttaaaatctagctcttcttatcactgtagcattattgcaatgaaaacacttcttaagtttacaagacatgactacatatttaaagacgtgttcagggaggttggccttttggaggtcatggtaaaccttttgcataaatatgctgccctgttgaaggatccaactcaggcactaaatgaacaaggggactcaagaaataatagttcagttgaagaccaaaaacacctggctttattggttatggagaccttgacagtgcttcttcaaggatcaaacacaaatgcaggaatttttcgagaatttggaggtgcaagatgtgcacataatatagtaaagtaccctcaatgccggcagcatgccttgatgactatccaacagctggtgctctccccaaatggggacgatgacatgggcactctcctggggctaatgcattcagccccaccgacggaattgcagttgaagactgatattttaagggccctcctgtcggtccttcgagaaagccatcgttcaagaacagtttttaggaaagttggaggatttgtgtacattacatccttgctcgttgctatggaaagatctttgagctgtccacccaagaatggctgggagaaagtgaaccagaatcaagtgtttgaacttcttcacactgtgttctgcacgttgactgcagcaatgcgctatgagccagccaactctcatttcttcaaaacagagattcagtatgagaagttggcagatgctgttcgatttcttggctgcttctcagacctaagaaaaataagcgccatgaatgtcttcccctcaaatacacagccatttcaaagacttttagaggaagatgtaatctcaatagaatcagtgtcacccacgttacggcactgcagtaaactttttatttatctttacaaagtagccacagattcttttgacagtcgtgcagaacagatccctccttgcctgacaagtgagtcttctctcccctctccttggggtacaccagctttgtccaggaaaaggcatgcatatcattctgtttcaactccccctgtttaccctcctaaaaatgttgccgacctgaaactacatgtgacaacttcatctctgcagagttctgatgcagtcatcattcatcctggagccatgcttgccatgctggacctactggcctctgttgggtcagtgacacagccagaacatgctttggatcttcaacttgccgtggcaaatattttacaatccctggtgcacacagaaaggaaccagcaagtcatgtgtgaagctggtcttcatgcacgactgctgcagaggtgcagtgctgcattggctgatgaggaccactcactgcacccgcccctgcagcggatgtttgaacgattagcctctcaggctctggaacccatggtgttgagggagtttttacgtttggcaagtcctttaaattgtggtgcctgggacaaaaaactgctaaaacaatatagggtccacaaaccaagttcactgagttatgaaccagaaatgagaagtagtatgatcacatctctggaaggtctgggtactgataatgtttttagcttacatgaagataaccattaccggataagcaagagcctggtaaaatctgcggaaggaagtactgtacccctgaccagggtgaagtgtctggtctccatgacaaccccacatgacatcagacttcatgggtcatcagttactccagcttttgttgaatttgacacatcacttgaagggtttggatgtctttttttgcccagtttggcccctcataatgctcctacaaataataccgtcacaacaggtcttattgatggggctgtggtcagtggcattggttctggtgaaagattcttccctcctccctccggcttaagttactctagctggttttgtattgaacattttagttctcctccaaataaccaccctgtcagacttcttactgttgtgcgccgagcaaattcttctgagcaacattacgtgtgccttgcaatagttctatcagcaaaagaccgatctctgattgtttccaccaaagaggaactcctccaaaattatgttgatgattttagtgaagagtcctcattttatgaaattctcccatgctgtgctcgctttcgatgtggagagcttatcattgagggacagtggcatcatttggtcctggtaatgagcaaaggcatgttgaaaaacagtactgcagccctttatattgatggacagcttgttaacactgtaaagcttcattatgtccacagtactccagggggttcaggttcggcaaatccaccagtggtgagcacggtctatgcctacattggtactccacctgcccaacgccaaattgcctcattggtttggcgcctgggacccacacattttctagaagaagttttaccttcttcaaatgttactaccatttatgaacttggaccaaattatgttggaagctttcaggctgtatgtatgccatgtaaagatgcaaaatccgaaggggtggtgccatcccctgtgtcattagtaccagaggagaaagtgtcatttggcctctatgcactctctgtgtcgtctctaacagtggcaagaatccggaaagtgtataacaaattggatagcaaagccattgctaagcagttaggcatttcctcacatgagaatgccactcctgtgaagttgatacacaattcagcaggacatcttaatggatctgcacggacaattggggccgctctgattggatacttgggagtaagaacatttgtccctaagcctgttgccactactttgcagtacgttggtggagctgcagccatcctgggcctggtggccatggcctctgatgtggaagggttatatgcagcagtcaaggccctggtttgtgtggtcaagagtaacccactagccagcaaagaaatggaaagaatcaagggctaccagttgctggcaatgttgcttaagaagaaacgttcccttcttaacagccacatcctccatctaactttttctttggtgggaactgttgatagtggacatgagacctccattattccaaattcaactgctttccaggacctcctctgtgattttgaagtctggctccatgcaccatatgaacttcatctttccttatttgaacactttattgaactgctcacagagtccagtgaagcctcaaagaatgccaaattaatgagagaattccagttaatcccaaagctgctcctgactcttcgagatatgtctttatcccagcctactattgctgctattagtaatgtcctgagcttcttactgcaaggttttcctagcagcaatgatctgctcagatttgggcagtttatttcttctactttgccaacctttgcggtttgtgagaaatttgtagtaatggaaataaataatgaagagaagcttgacactggaactgaagaggagtttggaggtcttgtatcagctaatcttatacttttgaggaacagacttctggatatcttgctaaaactaatttatacatctaaagaaaagacaagcattaatttgcaagcttgtgaagaactggtgaagacactgggttttgactggatcatgatgtttatggaggaacacttacattccaccacagttacagcagccatgaggattcttgttgtcctactaagtaatcagtctattctcatcaagtttaaagaaggactcagtggtggaggatggcttgaacagacagattctgtcttaactaataagattggaactgtattaggattcaacgtgggcagaagtgctggtgggagatcgacggtcagggagattaaccgagatgcttgtcattttcctggttttccagtccttcagtcattccttcctaaacacactaatgtccctgccctctattttctcctcatggccttgtttctgcagcagccagttagtgagctgcctgagaacctgcaggtcagtgtgcctgtcatcagctgccggagtaagcagggttgccagtttgatttggattccatttggacattcatctttggagttcctgcctccagcggaactgtggtctcttctatccataacgtatgcacagaagctgtttttttattattgggaatgctccgcagcatgctgacttcaccttggcaatcagaagaagagggatcttggctccgagaatatcctgtgaccctgatgcagttcttcagatatttgtatcacaacgtgccagaccttgcctccatgtggatgagccctgacttcctgtgtgcattagcagccaccgtcttccccttcaatattcgcccttactcagagatggtgactgaccttgatgatgaagttggatctccagcagaagagtttaaagcgtttgcagcagacacagggatgaacaggagccaatcagagtactgcaatgtgggcaccaagacatatctgaccaatcacccggctaaaaagttcgtttttgacttcatgcgggtcttaatcatagacaacctctgtctcactcctgccagcaagcaaactccactaattgatcttttgttggaggcttcccctgaaaggtctacaagaactcagcaaaaagaatttcaaacttacattttggatagcgtgatggaccatttgcttgcagctgatgtgttattaggggaagatgcatctctgcctattaccagtggaggaagctaccaggtattggtgaacaatgtgttttatttcacacagcgtgtggtggacaagctttggcaaggcatgttcaacaaagaatctaaacttcttatagattttataattcaactaattgcacagtcaaagagaagatcacagggattgtcactggatgcagtgtatcattgcctcaataggaccatcttgtaccagttctcacgggcacacaaaaccgttcctcagcaagtagctctgcttgattcactcagggtcctcactgtaaacagaaacttgatcctgggacctgggaaccatgaccaagaattcattagctgtctggcccactgcttgataaatctacatgttggaagcaacgtggatggatttggactggaagcagaagcccgcatgaccacatggcacattatgatcccctcggacattgaaccagatggtagttacagccaagatattagtgaagggcgtcagcttctcataaaagctgtcaacagagtttggactgaactgatacatagtaagaaacaagtcttagaggaacttttcaaagtaactctacctgtgaatgaaaggggccacgtggacatagctacagcaaggccactcattgaagaagctgccctgaagtgctggcagaatcatttggcccatgaaaagaaatgcataagtcgaggagaagctttagcgcccaccacacagtccaaattatcccgtgtcagcagtggctttggtctttccaagttaacaggatcaagaaggaatcgaaaagaaagtggtcttaataaacacagtctttccacccaggagatttcgcagtggatgtttactcacattgctgttgttcgtgacttagtagatacacaatataaagaatatcaggagcgtcagcagaatgccctgaagtacgtgacagaagagtggtgtcagatcgagtgcgagctgttgagggagcgggggctgtggggccctcccatcggctcccacctcgacaagtggatgctggagatgacagaagggccctgcaggatgaggaaaaagatggtgcgaaatgatatgttttataaccattacccttacgtgccagaaactgagcaagagacaaatgtggcgtctgagatcccaagtaaacagcctgagacacccgatgatattcctcaaaagaaacctgctcgatatagaagagccgtaagttatgacagtaaagagtactacatgcgactggcctctggcaatcccgccattgtccaagacgccattgtggagagttcagaaggtgaagctgctcagcaagaaccagagcatggggaagacactattgctaaagtcaaaggtttggtcaagcctcctctaaaacgctcccgatctgcacctgatggaggagatgaggagaaccaggagcagctacaagaccagattgctgagggcagctccatagaagaggaggagaaaacagataatgctaccttactgcgcctgttagaggaaggagaaaagatccaacacatgtaccgctgtgctcgagtccagggcctagataccagtgaggggctccttctttttggtaaagagcatttttatgtgattgatggatttaccatgacagcaaccagggaaataagagatattgaaaccttacctccaaatatgcatgagcctattattcctagaggagccaggcaaggccctagtcaactcaagagaacatgcagcatttttgcatatgaagatatcaaggaagttcataaaaggagatatctcctgcagcctattgctgtggaagttttctctggagatggacggaattacctccttgcttttcagaaaggaatcagaaacaaagtctatcaaaggtttttggctgtagtgccatctctaacggacagttcagaatctgtatctgggcaacgaccaaacacgagtgtggagcagggatctgggttacttagcactttggttggagagaagtctgtgactcagagatgggagagaggtgaaatcagcaacttccaatatttgatgcatttgaacactttggctggcagatcatataatgatctcatgcagtatcctgtcttcccctggatccttgcagattatgactcagaggaggtggatcttactaatcccaagacgtttagaaacctggctaagccaatgggagcacaaacagatgaacgattagctcagtataagaagcggtataaagactgggaggatcctaatggagaaactcctgcataccactatgggacccactattcatctgcaatgattgtggcctcataccttgtaaggatggagcctttcacacagatattcttaaggctacagggtggccactttgacctggctgaccggatgtttcacagtgtgcgcgaggcctggtattcagcgtcaaagcacaatatggcagatgtaaaagaacttatcccagagttcttttatttaccagaattcctgttcaattccaacaactttgatctaggctgtaaacaaaatggcaccaagcttggagatgttatccttccaccctgggcaaaaggggacccacgagaattcatcagagtccatcgtgaggctttggagtgtgattacgtgagtgcccatctacatgagtggattgacttaatcttcggttataaacagcaaggccctgctgcagtagaagctgtaaatgtcttccatcatcttttttatgagggtcaagtggatatctacaacatcaatgacccactaaaggagacagccacaattgggttcattaataacttcggtcagatccctaaacagttatttaaaaaacctcatccaccaaagcgagtgagaagtcgactcaatggagacaatgcaggaatctctgtcctaccaggatctacaagtgacaagatcttttttcatcatctagacaacttgaggccttctctaacacctgtaaaagaactcaaagaacctgtaggacaaatcgtatgtacagataaaggtattcttgcggtggaacagaataaggttcttatcccaccaacctggaataaaacttttgcttggggctatgcagacctcagttgcagactgggaacctatgagtcagacaaggccatgactgtttatgaatgcttgtctgagtggggccagattctctgtgcaatctgccccaaccccaagctggtcatcacgggtggaacaagcacggttgtgtgtgtgtgggagatgggcacctccaaagaaaaggccaagaccgtcaccctcaaacaggccttactgggccacactgataccgtcacctgcgccacagcatcattagcctatcacataattgtcagtgggtcccgtgatcgaacctgtatcatttgggatttgaacaaactgtcatttctaacccagcttcgagggcatcgagctccagtttctgctctttgtatcaatgaattaacaggggacattgtgtcctgcgctggcacatatatccatgtgtggagcatcaatgggaaccctatcgtgagtgtcaacacgttcacaggtaggagccagcagatcatctgctgctgcatgtcggagatgaacgaatgggacacgcagaacgtcatagtgacaggacactcagatggagtggttcggttttggagaatggaatttttgcaagttcctgaaacaccagctcctgagcctgctgaagtcctagaaatgcaggaagactgtccagaagcacaaatagggcaggaagcccaagacgaggacagcagtgattcagaagcagatgagcagagcatcagccaggaccctaaggacactccaagccaacccagcagcaccagccacaggccccgggcagcctcctgccgcgcaacagccgcctggtgtactgacagtggctctgacgactccagacgctggtccgaccagctcagtctagatgagaaagacggcttcatatttgtgaactattcagagggccagaccagagcccatctgcagggcccccttagccacccccaccccaatcccattgaggtgcggaattacagcagattgaaacctgggtaccgatgggaacggcagctggtgttcaggagtaagctgactatgcacacagcctttgatcgaaaggacaatgcacacccagctgaggtcactgccttgggcatctccaaggatcacagtaggatcctcgttggtgacagtcgaggccgagttttcagctggtctgtgagtgaccagccaggccgttctgctgctgatcactgggtgaaggatgaaggtggtgacagctgctcaggctgctcggtgaggttttcactcacagaaagacgacaccattgcaggaactgtggtcagctcttctgccagaagtgcagtcgctttcaatctgaaatcaaacgcttgaaaatctcatccccggtgcgtgtttgtcagaactgttattataacttacagcatgagagaggttcagaagatgggcctcgaaattgttgaagattcaacaagctgagtggagaccatggtctgtagaccccttcccgattctcctgtcccagcttggaaggcattgaaaacagtctccgtttacacatctcttcataccacgtgtttgaagtgttaaaattcaaagggatcattgaataaaacgggtgtagagtacaggaatggggcagacgcgattcaggtgaacagcacaagaagaatatgaggtggttcctaggagcaacactttcgacctccagttctccctgatgacagtagctgtctccaagagaaaaatcctcacttattaactctcttttcttgcatctcatttttatagagctactcatccttatttggaaaaaccaacaacaaaaaaggcttttagaaaatggttgtaaatctgacttctttgcaagtaactatgtatattgtaaatagatataaaaggccttttttctaaataaggacttaactgcctgtaacatgaaacttcaaactaaaccactaactcaatgaactacttatggtttgtctgacatccctcacttaccaattaattataaatatgtttttttaaatccccaaagacattatctgtggtctttttttcctttcaagctcagcctgtgtgcctgatgtcatttctttcaagttgcccacagtatctccacttaaactaggctagtaaccaaaataatgtggaccttctttaggaaacagtgtgggagaataggagtccagccgtaagataaactggaaatatttgggcgtcttgtacctggctacgcaccacctcagtgttgttcctacataaacagggccccttttaaacttgtatgtggactgctgtttggtcaaagaataccttcttagcattgcagaaaggtggtcagatgaccagtgtagtgcaggaaacagccctgtctcaactaatggaaatatatttgcatgtaacccaaaattagcttatcttgcatagaacataataagtatgtgtctttggtgacactaatgttctactatagcttattttcaaacaaggggtaaaaaaaggaaagaaagaagtgtacagaattaacatataaactttgttgtaaaactgaatcatgtcagaactgcttaaaattaacctttaccatttaatgtcatctacctgaaaacagtgagatttatactgtatcaatgtctatttttttgtttttgctatgaatataattacagtattttaatatttagttatttaatttgttctactagttggatacagaacacacaaatccagggggattaaagctggaaggggctaagagattagtttacagagaaaaggcttggtggtgggatttttttaaatgtgtgttatgtacatatatatatatatataatatatattaaaaatgaaacaattaatctagattttaacattttcagaaacttagtgataacattatgaacaattctaaaagccctgtgatttgaaaaatatagaatcattaatggcccaagataggccttcacaccttcacaggtgcgaaaggaaaggccttcacaccctcacagaggcatcatgcaaaggacagcggctttggcttttccaattttccatctttaggccctggtgagaggcacacttatgcactaaaatgcacatatatgcacatgcattcaaaaataggcatttggtacaatggtgatcttgtacctgatgggctgaaaccagcttaagaacaaatttgttcttcctgatatgataactaggtctccaagagaaaatagaaaggctgctttagtgccttacgcttactaaatttaaatctttatttacctgggtttgagcctacagtctatttatgattacatatcaaaattgattaaaacacttccatttctaaaagttcaaatatacttgttaataaaaggattatcggcattaatactttaatttaaagaaaagttgtgttctgttttcctttctgtgtcttactccccccacactctccctcccccatcaccatcttcaattctaataaataatgctgatgttcaacagttgcagaaattgtgctattatgtaactgtgggccttgcccctgtctggccctctagatgatttgtagcagtgttattctacactttttaaaagaagcgtcctccttttgtccatgaatcatgtttaccccatacccagtggcagaggtgttctttaaagacttgaatatatgaatgtgtgtgtgtagttacttaaaggttattcctctttgtaataggaaactatatgggatgaacacttttaaactttccgacacaacttccattactaactttctaacagaacttccataactagaaggtggaaaccaaaaccctcatggtagtatttcctctggcagctggtgctgtgggcaactgttttgttcaatcgggtttcttttctttttgcctctaatgcagaaatcaacagaatcactcacacatacaagtacactcacatacataaactaattatttctctggatatctttctgtgttccatgtaaatttatttaccaacatctattgtcaacatgtacatctaccttagtatggtctgcattctttttctgagagtacctcatagggctcctgcctgatctttgtagtttgttcattcatccatccacctgttcatttgttcatccatgtattctaacatttctatgtagtgtgcaactctaatgtcatgcttttgaagaagagaatagctgcccatagcagccatccgtctggataatagcaaaacactctagataagttattttgcactttcttatgtataaagttggtagaaacttatttttgctttgtatcatttaaatacattttgttttggtaaatgaactgtgtataaaatatttatgccgttaaaactgtttttagaaagtatttttaatttcagcaagtttggttacttgttgcatgactcttaacacagctgactttttgtgtcagtgcaatgtatattttttgtcctgttattaacttgtaagccctagtaatggccaattatttgtacagcaacagaagtaaattgaagatactggctaagactggattgattgtggacttttatactatattgcagaaaccaatatctgtttcttggtggttatgtaaaagacctgaagaattactatctagtgtgcagtctgtgatatctgaatgttcattgtatatttgtctctgatgcaaaaaggtagagtaa

SEQ ID NO: 10 - DNA, 20 bp, synthetic construct
  source 1..20: mol_type = other DNA
taatacgactcactataggg

SEQ ID NO: 11 - DNA, 18 bp, Homo sapiens
  source 1..18: mol_type = genomic DNA
aactcaaagaacctgtag

SEQ ID NO: 12 - DNA, 18 bp, Mus musculus
  source 1..18: mol_type = genomic DNA
aactcaaagagcctgtag

SEQ ID NO: 13 - DNA, 18 bp, Homo sapiens
  source 1..18: mol_type = genomic DNA
gacaaatcgtatgtacag

SEQ ID NO: 14 - DNA, 18 bp, Mus musculus
  source 1..18: mol_type = genomic DNA
gacaaatcgtgtgcacgg

SEQ ID NO: 15 - DNA, 19 bp, Homo sapiens
  source 1..19: mol_type = genomic DNA
ataaaggtattcttgcggt

SEQ ID NO: 16 - DNA, 19 bp, Mus musculus
  source 1..19: mol_type = genomic DNA
ataaaggcattctcgccgt

SEQ ID NO: 17 - DNA, 19 bp, Homo sapiens
  source 1..19: mol_type = genomic DNA
ggaacagaataaggttctt

SEQ ID NO: 18 - DNA, 19 bp, Mus musculus
  source 1..19: mol_type = genomic DNA
ggagcagaacaaggtactc

SEQ ID NO: 19 - DNA, 19 bp, Homo sapiens
  source 1..19: mol_type = genomic DNA
atcccaccaacctggaata

SEQ ID NO: 20 - DNA, 19 bp, Mus musculus
  source 1..19: mol_type = genomic DNA
atcccgcccgcgtggaaca

SEQ ID NO: 21 - DNA, 30 bp, Homo sapiens
  source 1..30: mol_type = genomic DNA
gataaaggtattcttgcggtggaacagaat

SEQ ID NO: 22 - DNA, 30 bp, Homo sapiens
  source 1..30: mol_type = genomic DNA
attctgttccaccgcaagaatacctttatc

SEQ ID NO: 23 - DNA, 20 bp, synthetic construct
  source 1..20: mol_type = other DNA
aggtattcttgcggtggaac

SEQ ID NO: 24 - DNA, 34 bp, synthetic construct
  source 1..34: mol_type = other DNA
gataaaggtgttcttgcggtggaacagaataagg

SEQ ID NO: 25 - DNA, 34 bp, synthetic construct
  source 1..34: mol_type = other DNA
ccttattctgttccaccgcaagaacacctttatc

SEQ ID NO: 26 - DNA, 65 bp, Homo sapiens
  source 1..65: mol_type = genomic DNA
cctgtaggacaaatcgtatgtacagataaaggtattcttgcggtggaacagaataaggttcttat

SEQ ID NO: 27 - DNA, 65 bp, synthetic construct
  source 1..65: mol_type = other DNA
cctgtaggacaaatcgtatgtacagataaaggtgttcttgcggtggaacagaataaggttcttat